(19) United States
(12) Patent Application Publication
(10) Pub. No.: US 2022/0249660 A1
(43) Pub. Date: Aug. 11, 2022

(54) COMPOSITIONS AND METHODS FOR TREATING LUNG, COLORECTAL AND BREAST CANCER

(71) Applicant: Sitokine Limited, London (GB)

(72) Inventors: Kenneth S. KORNMAN, Newton, MA (US); Lynn DOUCETTE-STAMM, Framingham, MA (US); Gordon W. DUFF, Sheffield (GB)

(21) Appl. No.: 17/616,526

(22) PCT Filed: Jun. 5, 2020
(86) PCT No.: PCT/EP2020/065692

§ 371 Date: Dec. 3, 2021

Related U.S. Application Data
(60) Provisional application No. 62/858,147, filed on Jun. 6, 2019.

(51) Int. Cl. A61K 39/395; G01N 33/574; C12Q 1/6886; C07K 16/24; A61K 31/325; A61K 31/337; A61K 31/704; A61K 31/517; A61K 31/444; A61K 38/12; A61K 38/14; A61K 31/136; A61K 31/4745; A61K 31/4545; A61K 31/519; A61K 31/4184; A61K 31/4706; A61K 31/404; A61K 31/506; A61K 31/407; A61K 31/475; A61K 31/7048; A61K 31/675; A61K 31/196; A61K 31/513; A61K 31/436; A61K 31/7068; A61K 31/5377; A61P 35/00

(52) U.S. Cl. CPC A61K 39/3955; G01N 2800/50; C12Q 1/6886; G01N 33/57419; C07K 16/245; C07K 16/248; A61K 31/325; A61K 31/337; A61K 31/704; A61K 31/517; A61K 31/444; A61K 38/12; A61K 38/14; A61K 31/136; A61K 31/4745; A61K 31/4545; A61K 31/519; A61K 31/4184; A61K 31/4706; A61K 31/404; A61K 31/506; A61K 31/407; A61K 31/475; A61K 31/7048; A61K 31/675; A61K 31/196; A61K 31/513; A61K 31/436; A61K 31/7068; A61K 31/5377; A61P 35/00; C12Q 2600/156; C12Q 2600/118; G01N 33/57423

(57) ABSTRACT

The disclosure relates to compositions and methods for reducing the risk of developing a lung cancer, colorectal cancer, or metastatic breast cancer in a subject, and methods of treating the same, comprising identifying a subject who has one or more risk factors for lung, colorectal or metastatic breast cancer and carries IL-1 single nucleotide polymorphisms (SNPs) associated with high levels of inflammation.

### CROSS REFERENCE TO RELATED APPLICATIONS

This application claims priority to U.S. provisional application No. 62/858,147, filed on Jun. 6, 2019, the contents of which are incorporated by reference in their entirety herein.

### FIELD OF THE INVENTION

The invention relates to the prevention and treatment of cancers such as lung cancers, colorectal cancers and breast cancers, and genetic testing for elevated inflammation associated with cancer.

### BACKGROUND OF THE INVENTION

According to the National Institutes of Health, lung, colorectal and breast cancers are three of the most common cancers. In the U.S. alone, 143,000 people are expected to die of lung cancer, 42,000 are expected to die of breast cancer, and 51,000 are expected to die of colorectal cancer per year. There thus exists a pressing need in the art for additional therapies for the prevention and treatment of lung cancer, breast cancer and colorectal cancer.

### SUMMARY OF THE INVENTION

The disclosure provides methods of reducing a risk of, or treating, lung cancer, colorectal cancer or metastatic breast cancer in a subject, comprising: (a) identifying a subject who has, or who is at risk of developing, lung cancer colorectal cancer or breast cancer; (b) obtaining information regarding the subject's single nucleotide polymorphism (SNP) alleles for any of the 3 or 5 SNP combinations disclosed in Tables 1-3; (c) diagnosing the subject as at risk for lung, colorectal, or metastatic breast cancer if the subject has a positive IL-1 genotype pattern obtained in (b) that is the same as any of any of those disclosed as IL-1 positive genotypes in tables 1-3; and (d) administering an inflammation inhibitor to the subject.

The disclosure provides methods of reducing a risk of developing lung cancer in a subject comprising: (a) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (b) diagnosing the subject as at risk of developing lung cancer if the subject has a positive IL-1 genotype pattern obtained in (b) that is the same as any of the IL-1 positive genotype patterns described in tables 1 and 2; and (c) administering a non-genetic lung cancer test to the subject diagnosed as having an IL-1 positive genotype pattern in step (b).

The disclosure provides methods of reducing a risk of developing lung cancer in a subject comprising: (a) obtaining information regarding the subject's single nucleotide polymorphism (SNP) alleles for each of the rs16944 polymorphic locus, the rs1143623 polymorphic locus and the rs4848306 polymorphic locus; (b) diagnosing the subject as at risk of developing lung cancer if the subject has a positive IL-1 genotype pattern obtained in (b) that is the same as any of the IL-1 positive genotype patterns described in table 3; and (c) administering a non-genetic lung cancer test to the subject diagnosed as having an IL-1 positive genotype pattern in step (b).

In some embodiments of the methods of the disclosure, the subject does not have a risk factor for lung cancer.

In some embodiments of the methods of the disclosure, the methods comprise identifying a subject who has a risk factor for lung cancer prior to step (a). In some embodiments, the risk factor comprises an environmental risk factor, a genetic risk factor, a biomarker, a previous history of lung cancer, or lung nodules or masses. In some embodiments, the environmental risk factor comprises a smoking history, exposure to second hand smoke, asbestos, radon or diesel exhaust, inhalation of carcinogenic chemicals or radioactive materials or previous radiation therapy directed to the thorax. In some embodiments, the biomarker comprises an angiogenic factor, a lung cancer associated protein, an RNA, a DNA, a micro-RNA, an exosome, a circulating tumor cell or a change in metabolites. In some embodiments, the genetic risk factor comprises a family history of lung cancer. In some embodiments, the smoking history comprises less than 30 pack years. In some embodiments, the smoking history comprises more than 15 years since quitting smoking. In some embodiments, the subject is less than 55 or greater than 80 years of age. In some embodiments, the non-genetic lung cancer test comprises an imaging test or a test for a lung cancer biomarker. In some embodiments, the imaging test comprises a chest X-ray, sputum cytology, magnetic resonance imaging (MRI) or fluorodeoxyglucose positron emission tomography computed tomography (PET/CT). In some embodiments, the chest X-ray comprises a low-dose computed tomography (CT) scan for lung cancer or suspicious nodules, or a low-dose helical CT scan.

In some embodiments of the methods of the disclosure, the test for a lung cancer biomarker comprises testing for an angiogenic factor, one or more proteins associated with lung cancer, RNA, DNA, micro-RNA, exosome, circulating tumor cells or a change in metabolites associated with lung cancer. In some embodiments, the one or more protein associated with lung cancer comprises AGER, C10orf116, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, creatine kinase, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, plasminogen activator, UPA, MT1-MMP, SFN, TF, ALB, S100A9, STMN, ENO, IGFBP7, or THBS1.

In some embodiments of the methods of the disclosure, the testing is administered once a month, every 2 months, every 3 months, every 4 months, every 5 months, every 6 months, every 8 months, every 12 months, every 18 months, every 2 years, every 2.5 years or every 3 years.

In some embodiments of the methods of the disclosure, the methods further comprise administering an inflammation inhibitor to the subject. In some embodiments, the inflammation inhibitor is formulated as an aerosol. In some embodiments, the aerosol is administered as a nasal spray. In some embodiments, the inflammation inhibitor is an IL-1 inhibitor or an IL-6 inhibitor. In some embodiments, the inflammation inhibitor is an inhibitor of an IL-1 driven inflammatory mediator. In some embodiments, the inhibitor of an IL-1 driven inflammatory mediator is a GM-CSF inhibitor or a JAK/STAT inhibitor. In some embodiments, the IL-1 inhibitor is an IL-1α inhibitor or an IL-1β inhibitor. In some embodiments, the IL-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, Canakinumab, a DOM4-130-201 and DOM4-130-202 antibody. In some embodiments, the IL-1β inhibitor is Canakinumab or a derivative thereof. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the IL-6 inhibitor is selected from the group consisting of Tocilizumab, Siltuximab, Olokizumab, Elsilimomab, Sirukimab, Levilimab, ALX-0061, Gerilimzumab and Sarilumab.

The disclosure further provides methods of treating lung cancer in a subject comprising: (a) identifying a subject who has lung cancer; (b) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in tables 1 and 2; and (d) administering an inflammation inhibitor to the subject diagnosed as having a positive IL-1 genotype pattern in (c).

The disclosure further provides methods of treating lung cancer in a subject comprising: (a) identifying a subject who has lung cancer; (b) obtaining information regarding the subject's single nucleotide polymorphism (SNP) alleles for each of the rs16944 polymorphic locus, the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in table 3; and (d) administering an inflammation inhibitor to the subject diagnosed as having a positive IL-1 genotype pattern in (c).

The disclosure further provides methods of treating lung cancer in a subject comprising: (a) identifying a subject who has a high risk for lung cancer based on age, family history of lung cancer, smoking history, or history of environmental exposure to smoke; as described by one of the current guidelines for lung cancer risk (www.cdc.gov/cancer/lung/pdf/guidelines.pdf); (b) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in tables 1 and 2; (d) administering low-dose computed tomography (CT) screening for lung cancer or suspicious nodules if subject is diagnosed as having a positive IL-1 genotype pattern in (c); and (e) administering an inflammation inhibitor to the subject diagnosed as having a positive IL-1 genotype pattern in (c) and a positive screening assessment for lung cancer or suspicious nodules in (d).

The disclosure further provides methods of treating lung cancer in a subject comprising: (a) identifying a subject who has a high risk for lung cancer based on age, family history of lung cancer, smoking history, or history of environmental exposure to smoke; (b) obtaining information regarding the subject's single nucleotide polymorphism (SNP) alleles for each of the rs16944 polymorphic locus, the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in table 3; (d) administering low-dose computed tomography (CT) screening for lung cancer or suspicious nodules if subject is diagnosed as having a positive IL-1 genotype pattern in (c); and (e) administering an inflammation inhibitor to the subject diagnosed as having a positive IL-1 genotype pattern in (c) and a positive screening assessment for lung cancer or suspicious nodules in (d).

In some embodiments of the methods of the disclosure, identifying a subject who has lung cancer comprises: (i) identifying a subject who has one or more risk factors or biomarkers of lung cancer; and (ii) testing the subject for lung cancer.

In some embodiments of the methods of the disclosure, the risk factor comprises an environmental risk factor, a genetic risk factor, a biomarker, a previous history of lung cancer, or lung nodules or masses. In some embodiments, the environmental risk factor comprises a smoking history, exposure to second hand smoke, asbestos, radon or diesel exhaust, inhalation of carcinogenic chemicals or radioactive materials or previous radiation therapy directed to the thorax. In some embodiments, the genetic risk factor comprises a family history of lung cancer. In some embodiments, the biomarker comprises an angiogenic factor, one or more lung cancer associated proteins, an RNA, a DNA, a micro-RNA, an exosome, a circulating tumor cell or a change in metabolites. In some embodiments, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, (SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, UPA, MT1-MMP, SFN, TF, ALB, S100A9, STMN, ENO, IGFBP7, or THBS1. In some embodiments, the risk factor comprises a lung nodule, lung tumor, lung mass, evidence of angiogenesis or evidence of tumor invasion of other tissues.

In some embodiments of the methods of the disclosure, testing the subject for lung cancer comprises a biopsy of a lung tumor.

In some embodiments of the methods of the disclosure, the methods comprise administering an inflammation inhibitor to the subject. In some embodiments, the inflammation inhibitor is formulated as an aerosol. In some embodiments, the aerosol is administered as a nasal spray. In some embodiments, the inflammation inhibitor is an IL-1 inhibitor or an IL-6 inhibitor. In some embodiments, the inflammation inhibitor is an inhibitor of an IL-1 driven inflammatory mediator. In some embodiments, the inhibitor of an IL-1 driven inflammatory mediator is a GM-CSF inhibitor or a JAK/STAT inhibitor. In some embodiments, IL-1 inhibitor is an IL-1β inhibitor or an IL-1α inhibitor. In some embodiments, the L-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201, a DOM4-130-202 antibody and Canakinumab. In some embodiments, the IL-1β inhibitor is Canakinumab or a derivative thereof. In some embodiments, the Canakinumab is administered to the subject at a dose of 25 mg to 300 mg. In some embodiments, the subject weighs less than 40 kg and the Canakinumab is administered to the subject at a dose of 2 mg/kg or 4 mg/kg. In some embodiments, the subject weighs more than 40 kg and the Canakinumab is administered to the subject at a dose of 150 mg or 300 mg. In some embodiments, the Canakinumab is administered every 2 weeks, every 4 weeks, every 6 weeks, every 8 weeks, every 10 weeks, every 3 months, every 5 months or every 6 months from the first administration. In some embodiments, the Canakinumab is administered every 4 weeks from the first administration. In some embodiments, the Canakinumab is administered parenterally. In some embodiments, the Canakinumab is administered by intravenous injection, intravenous infusion, intramuscularly, via intrapulmonary administration or subcutaneously. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the IL-6 inhibitor is selected from the group consisting of Tocilizumab, Siltuximab, Olokizumab, Elsilimomab, Sirukimab, Levilimab, ALX-0061, Gerilimzumab and Sarilumab.

In some embodiments of the methods of the disclosure, the lung cancer is stage 0, stage 1, stage 2, stage 3 or stage 4 lung cancer.

In some embodiments of the methods of the disclosure, administering the inflammation inhibitor reduces a sign or a symptom of the cancer. In some embodiments, administering the inflammation inhibitor reduces a number of tumors of the cancer, reduces a size of a tumor of the cancer, reduces a growth rate of a tumor of the cancer, reduces early metaplastic changes in the cancer, reduces neo-angiogenesis, reduces tissue invasiveness by the cancer, reduces tissue invasion by the cancer through a basement membrane, reduces invasion of bone by the cancer, reduces metastasis of the cancer to distant organs, or a combination thereof. In some embodiments, administering the inflammation inhibitor reduces a level of one or more biomarkers associated with lung cancer. In some embodiments, the biomarker comprises AGER, C10orf116, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, (SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, UPA, MT1-MMP, SFN, TF, ALB, S100A9, STMN, ENO, IGFBP7, or THBS1.

The disclosure provides methods of reducing a risk of developing colorectal cancer in a subject comprising: (a) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (b) diagnosing the subject as at risk of developing colorectal cancer if the subject has a positive IL-1 genotype pattern obtained in (b) that is the same as any of the IL-1 positive genotype patterns described in tables 1 and 2; and (c) administering a non-genetic colorectal cancer test to the subject diagnosed as having an IL-1 positive genotype pattern in step (b).

The disclosure provides methods of reducing a risk of developing colorectal cancer in a subject comprising: (a) obtaining information regarding the subject's single nucleotide polymorphism (SNP) alleles for each of the rs16944 polymorphic locus, the rs1143623 polymorphic locus and the rs4848306 polymorphic locus; (b) diagnosing the subject as at risk of developing colorectal cancer if the subject has a positive IL-1 genotype pattern obtained in (b) that is the same as any of the IL-1 positive genotype patterns described in table 3; and (c) administering a non-genetic lung or colorectal cancer test to the subject diagnosed as having an IL-1 positive genotype pattern in step (b).

In some embodiments of the methods of the disclosure, the subject does not have a risk factor for colorectal cancer.

In some embodiments of the methods of the disclosure, the subject has one or more risk factors for colorectal cancer. In some embodiments, the one or more risk factors comprise being overweight or obese, lack of physical activity, diet, smoking, heavy alcohol use, age over fifty, a history of adenomatous polyps, a family history of adenomatous polyps, a previous diagnosis of colorectal cancer, a family history of colorectal cancer, a history of inflammatory bowel disease, type II diabetes, radiation therapy to treat prostate cancer or a genetic predisposition to colorectal cancer. In some embodiments, the genetic predisposition to colorectal cancer comprises Lynch syndrome, familial adenomatous polyposis (FAP), or a mutation in LBK1, MUTYH or SMAD4. In some embodiments, the Lynch syndrome comprises MLH1 mutation or a MSH2 mutation. In some embodiments, the FAP comprises a mutation in the adenomatous polyposis coli (APC) gene. In some embodiments, the diet comprises a diet high in red meat or processed meat, or both.

In some embodiments of the methods of the disclosure, the non-genetic colorectal cancer test comprises a high-sensitivity fecal occult blood test (FOBT), a fecal immunochemical test (FIT), a sigmoidoscopy, a colonoscopy, computed tomographic (CT) colonography, a double contrast barium enema or a blood test. In some embodiments, the FIT tests for at least one DNA marker associated with colorectal cancer. In some embodiments, the at least one DNA marker associated with colorectal cancer comprises an alteration in APC, CTNNB1, KRAS, BRAF, SMAD4, TGFBR2, TP53, PIK3CA, ARID1A, SOX9, FAM123B, ERBB2, VIM, NDRG4, SEPT9, BMP) or TFPI2. In some embodiments, the alteration comprises a mutation or a change in DNA methylation. In some embodiments, the FIT or FOBT comprises an immunoassay for hemoglobin. In some embodiments, the blood test comprises testing for a biomarker associated with colorectal cancer. In some embodiments, the biomarker comprises methylated SEPT9 DNA.

In some embodiments of the methods of the disclosure, the testing is administered once a month, every 2 months, every 3 months, every 4 months, every 5 months, every 6 months, every 8 months, every 12 months, every 18 months, every 2 years, every 2.5 years or every 3 years.

In some embodiments of the methods of the disclosure, the method further comprises administering an inflammation inhibitor. In some embodiments, the inflammation inhibitor is an inhibitor of an IL-1 driven inflammatory mediator. In some embodiments, the inhibitor of an IL-1 driven inflammatory mediator is a GM-CSF inhibitor or a JAK/STAT inhibitor. In some embodiments, the inflammation inhibitor is an IL-1 inhibitor or an IL-6 inhibitor. In some embodiments, IL-1 inhibitor is an IL-1α inhibitor or an IL-1β inhibitor. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the IL-1α inhibitor is Bermekimab. In some embodiments, the IL-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, Canakinumab, a DOM4-130-201 and DOM4-130-202 antibody. In some embodiments, the IL-6 inhibitor is selected from the group consisting of Tocilizumab, Siltuximab, Olokizumab, Elsilimomab, Sirukimab, Levilimab, ALX-0061, Gerilimzumab and Sarilumab.

The disclosure further provides methods of treating colorectal cancer in a subject comprising: (a) identifying a subject who has colorectal cancer; (b) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in tables 1 and 2; (d) administering an inflammation inhibitor to the subject to the subject.

The disclosure further provides methods of treating colorectal cancer in a subject comprising: (a) identifying a subject who has colorectal cancer; (b) obtaining information regarding the subject's single nucleotide polymorphism (SNP) alleles for each of the rs16944 polymorphic locus, the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in table 3; (d) administering an inflammation inhibitor to the subject to the subject.

The disclosure further provides methods of treating colorectal cancer in a subject comprising: (a) identifying a subject who has colorectal cancer; (b) obtaining information regarding the subject's single nucleotide polymorphism (SNP) alleles for e each of the rs16944 polymorphic locus, the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in tables 1 and 2; (d) administering an inflammation inhibitor to the subject to the subject.

The disclosure further provides methods of treating colorectal cancer in a subject comprising: (a) identifying a subject who has a high risk for colorectal cancer based on age, obesity, diets high in red meat or meats cooked at very high temperature, smoking, heavy alcohol use, personal history of colorectal polyps or colorectal cancer, having a family history of hereditary non-polyposis colon cancer; as described by one of the current guidelines for colorectal cancer risk (www.cancer.org/cancer/colon-rectal-cancer/causes-risks-prevention/risk-factors.html); (b) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in tables 1 and 2; (d) if subject is diagnosed as having a positive IL-1 genotype pattern in (c) administering a colonoscopy screening for colon cancer or suspicious polyps; and (e) administering an inflammation inhibitor to the subject diagnosed as having a positive IL-1 genotype pattern in (c) and a positive screening assessment for colon cancer or suspicious nodules in (d).

The disclosure further provides methods of treating colorectal cancer in a subject comprising: (a) identifying a subject who has a high risk for colorectal cancer based on age, obesity, diets high in red meat or meats cooked at very high temperature, smoking, heavy alcohol use, personal history of colorectal polyps or colorectal cancer, having a family history of hereditary non-polyposis colon cancer; (b) obtaining information regarding the subject's single nucleotide polymorphism (SNP) alleles for each of the rs16944 polymorphic locus, the rs1143623 polymorphic locus and the rs4848306 polymorphic locus; (c) diagnosing the subject as having a positive IL-1 genotype pattern if the SNP alleles obtained in (b) are the same as any of the IL-1 positive genotype patterns described in table 3; (d) if subject is diagnosed as having a positive IL-1 genotype pattern in (c) administering a colonoscopy screening for colon cancer or suspicious polyps; and (e) administering an inflammation inhibitor to the subject diagnosed as having a positive IL-1 genotype pattern in (c) and a positive screening assessment for colon cancer or suspicious nodules in (d).

In some embodiments of the methods of the disclosure, identifying a subject who has colorectal cancer comprises: (i) identifying a subject who has one or more risk factors or biomarkers of colorectal cancer; and (ii) testing the subject for colorectal cancer.

In some embodiments of the methods of the disclosure, the one or more risk factors comprise being overweight or obese, lack of physical activity, diet, smoking, heavy alcohol use, age over fifty, a personal history of adenomatous polyps, a family history of adenomatous polyps or hereditary non-polyposis colon cancer, a previous diagnosis of colorectal cancer, a family history of colorectal cancer, a history of inflammatory bowel disease, type II diabetes, radiation therapy to treat prostate cancer, a genetic predisposition to colorectal cancer or one testing positive for one or more indicators of colorectal cancer. In some embodiments, the genetic predisposition to colorectal cancer comprises Lynch syndrome, familial adenomatous polyposis (FAP), or a mutation in LBK1, MUTYH or SMAD4. In some embodiments, the Lynch syndrome comprises an MLH1 mutation or an MSH2 mutation. In some embodiments, the FAP comprises a mutation in the adenomatous polyposis coli (APC) gene. In some embodiments, the diet comprises a diet high in red meat, processed meat, meat cooked at high temperature, or a combination thereof. In some embodiments, the biomarker for colorectal cancer is assayed using a high-sensitivity fecal occult blood test (FOBT), a fecal immunochemical test (FIT), a sigmoidoscopy, a colonoscopy screening for colon cancer or suspicious polyps, computed tomographic (CT) colonography, a double contrast barium enema or a blood test. In some embodiments, the FIT tests for at least one DNA marker associated with colorectal cancer. In some embodiments, the at least one DNA marker associated with colorectal cancer comprises an a mutation or a change in methylation in APC, CTNNB1, KRAS, BRAF, SMAD4, TGFBR2, TP53, PIK3CA, ARID1A, SOX9, FAM123B, ERBB2, VIM, NDRG4, SEPT9, BMP3 or TFPI2. In some embodiments, the biomarker comprises methylated SEPT9 DNA. In some embodiments, the FIT or the FOBT comprises an immunoassay for hemoglobin.

In some embodiments of the methods of the disclosure, testing the subject for colorectal cancer comprises a biopsy.

In some embodiments of the methods of the disclosure, the inflammation inhibitor is an IL-1 inhibitor or an IL-6 inhibitor. In some embodiments, the inflammation inhibitor is an inhibitor of an IL-1 driven inflammatory mediator. In some embodiments, the inhibitor of an IL-1 driven inflammatory mediator is a GM-CSF inhibitor or a JAK/STAT inhibitor. In some embodiments, the inflammation inhibitor is formulated as an aerosol. In some embodiments, the aerosol is administered as a nasal spray. In some embodiments, the IL-1 inhibitor is an IL-1β inhibitor or an IL-1α inhibitor. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the IL-1α inhibitor is Bermekimab. In some embodiments, the Bermekimab is administered at between 3 mg/kg to 20 mg/kg. In some embodiments, the Bermekimab is administered at 7.5 mg/kg. In some embodiments, the Bermekimab is administered parenterally. In some embodiments, the parenteral administration comprises subcutaneous injection, intramuscular injection, intravenous injection or intravenous infusion. In some embodiments, the Bermekimab is administered every week, every two weeks, every three weeks, every 4 weeks, every 5 weeks, every 6 weeks or every 8 weeks. In some embodiments, the L-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201, a DOM4-130-202 antibody and Canakinumab. In some embodiments, the IL-1β inhibitor is Canakinumab or a derivative thereof. In some embodiments, the IL-6 inhibitor is selected from the group consisting of Tocilizumab, Siltuximab, Olokizumab, Elsilimomab, Sirukimab, Levilimab, ALX-0061, Gerilimzumab and Sarilumab.

In some embodiments of the methods of the disclosure, the colorectal cancer is stage 0, stage 1, stage 2, stage 3 or stage 4 colorectal cancer.

In some embodiments of the methods of the disclosure, administering the inflammation inhibitor reduces a sign or a symptom of the colorectal cancer. In some embodiments, administering the inflammation inhibitor reduces a number of tumors of the cancer, reduces a size of a tumor of the cancer, reduces a growth rate of a tumor of the cancer, reduces early metaplastic changes in the cancer, reduces neo-angiogenesis, reduces tissue invasiveness by the cancer, reduces tissue invasion by the cancer through a basement membrane, reduces invasion of bone by the cancer, reduces metastasis of the cancer to distant organs, or a combination thereof. In some embodiments, administering the inflammation inhibitor reduces one or more biomarkers associated with colorectal cancer. In some embodiments, the biomarker comprises a mutation or change in methylation state of APC, CTNNB1, KRAS, BRAF, SMAD4, TGFBR2, TP53, PIK3CA, ARID1A, SOX9, FAM123B, ERBB2, VIM, NDRG4, SEPT9, BMP3 or TFPI2.

The disclosure further provides methods of reducing a risk of metastatic breast cancer in a subject comprising: (a) identifying a subject who has breast cancer with no evidence of metastasis; (b) obtaining information regarding the subject's single nucleotide polymorphism (SNP) alleles for each of the rs17561 polymorphic locus, the rs16944 polymorphic locus and the rs1143634 polymorphic locus, and optionally further obtaining information regarding the subject's SNPs at the rs1143623 polymorphic locus and the rs4848306 polymorphic locus; (c) diagnosing the subject as at risk for metastatic breast cancer if the subject has a positive IL-1 genotype pattern obtained in (b) that is the same as any of the IL-1 positive genotype patterns described in tables 1 and 2; and (d) administering an inflammation inhibitor to the subject diagnosed with a positive IL-1 genotype pattern in step (c).

The disclosure further provides methods of reducing a risk of metastatic breast cancer in a subject comprising: (a) identifying a subject who has breast cancer with no evidence of metastasis; (b) obtaining information regarding the subject's single nucleotide polymorphism (SNP) alleles for each of the rs16944 polymorphic locus, the rs1143623 polymorphic locus and the rs4848306 polymorphic locus; (c) diagnosing the subject as at risk for metastatic breast cancer if the subject has a positive IL-1 genotype pattern obtained in (b) that is the same as any of the IL-1 positive genotype patterns described in table 3; and (d) administering an inflammation inhibitor to the subject diagnosed with a positive IL-1 genotype pattern in step (c).

In some embodiments of the methods of the disclosure, the breast cancer comprises a carcinoma, a sarcoma, a Phyllodes tumor, Paget disease, an angiosarcoma or an inflammatory breast cancer. In some embodiments, the breast cancer is a pre-metastatic stage 0, stage I, stage II or stage III breast cancer.

In some embodiments of the methods of the disclosure, the inflammation inhibitor is an IL-1 inhibitor or an IL-6 inhibitor. In some embodiments, the inflammation inhibitor is an inhibitor of an IL-1 driven inflammatory mediator. In some embodiments, the inhibitor of an IL-1 driven inflammatory mediator is a GM-CSF inhibitor or a JAK/STAT inhibitor. In some embodiments, the IL-1 inhibitor is an IL-1α inhibitor or an IL-1β inhibitor. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the L-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201 antibody, DOM4-130-202 antibody and Canakinumab. In some embodiments, the IL-1β inhibitor is Canakinumab or a derivative thereof. In some embodiments, the Canakinumab is administered to the subject at a dose of 25 mg to 300 mg. In some embodiments, the subject weighs less than 40 kg and the Canakinumab is administered to the subject at a dose of 2 mg/kg or 4 mg/kg. In some embodiments, subject weighs more than 40 kg and the Canakinumab is administered to the subject at a dose of 150 mg or 300 mg. In some embodiments, the Canakinumab is administered every 2 weeks, every 4 weeks, every 6 weeks, every 8 weeks, every 10 weeks, every 3 months, every 5 months or every 6 months from the first administration. In some embodiments, the Canakinumab is administered every 4 weeks from the first administration. In some embodiments, the Canakinumab is administered parenterally. In some embodiments, the Canakinumab is administered by intravenous injection, intravenous infusion, intramuscularly or subcutaneously. In some embodiments, the IL-6 inhibitor is selected from the group consisting of Tocilizumab, Siltuximab, Olokizumab, Elsilimomab, Sirukimab, Levilimab, ALX-0061, Gerilimzumab and Sarilumab.

In some embodiments of the methods of the disclosure the IL-1 inhibitor is an IL-1α inhibitor or an IL-1β inhibitor. In some embodiments, the IL-1α inhibitor is selected from the group consisting of Bermekimab, ABT-981, Isunakinra, AC-701, Sairei-To, Can-04, XL-130, a MABp1 antibody and Givinostat. In some embodiments, the IL-1α inhibitor is Bermekimab. In some embodiments, the Bermekimab is administered at between 3 mg/kg to 20 mg/kg. In some embodiments, the Bermekimab is administered at 7.5 mg/kg. In some embodiments, the Bermekimab is administered parenterally. In some embodiments, the parenteral administration comprises subcutaneous injection, intramuscular injection, intravenous injection or intravenous infusion. In some embodiments, the Bermekimab is administered every week, every two weeks, every three weeks, every 4 weeks, every 5 weeks, every 6 weeks or every 8 weeks.

In some embodiments of the methods of the disclosure, administering the inflammation inhibitor reduces a sign or a symptom of the breast cancer. In some embodiments, administering the inflammation inhibitor reduces metastasis of the breast cancer.

In some embodiments of the methods of the disclosure, the methods further comprise chemotherapy, radiation treatment, surgical removal of the cancer, an immunotherapy, an immune checkpoint inhibitor, a therapeutic vaccine, an antibody therapy, or a combination thereof. In some embodiments of the methods of the disclosure, the chemotherapy comprises a taxane, a platinum agent, an alkylating agent, a mitotic inhibitor, an antimetabolite, an alkaloid, an antitumor antibiotic, a topoisomerase inhibitor, a tyrosine kinase inhibitor, an mTOR inhibitor, a B-Raf inhibitor, an EGFR inhibitor, a PARP inhibitor, a phosphoinositide 3-kinase (PI3K) inhibitor, a CDK inhibitor or a combination thereof. In some embodiments, the topoisomerase inhibitor comprises doxorubicin, epirubicin, irinotecan, topotecan, mitoxantrone, daunorubicin or etoposide. In some embodiments, the alkylating agent comprises cyclophosphamide, chlorambucil, melphalan, ifosfamide or mechlorethamine hydrochloride. In some embodiments, the antimetabolite comprises pemetrexed, gemcitabine, methotrexate, 5-fluorouracil, capecitabine or trifluridine and tipiracil. In some embodiments, the alkaloid comprises actinomycin D, doxorubicin or mitomycin, vinorelbine or vinblastine. In some embodiments, the antitumor antibiotic comprises doxorubicin, mitoxantrone or bleomycin. In some embodiments, the taxane comprises taxol, docetaxel or paclitaxel. In some embodiments, the tyrosine kinase inhibitor comprises afatinib, apatinib, alectinib, brigantinib, ceritinib, CDX-301, crizotinib, trametinib, selumetinib, lapatinib, neratinib or sunitinib. In some embodiments, the mTOR inhibitor comprises everolimus. In some embodiments, the platinum agent comprises cisplatin, oxaliplatin or carboplatin. In some embodiments, the B-Raf inhibitor comprises dabrafenib. In some embodiments, the EGFR inhibitor comprises erlotinib, gefetinib or osimertinib. In some embodiments, the PARP inhibitor comprises veliparib, olaparib or talazoparib. In some embodiments, the PI3K inhibitor comprises buparlisib. In some embodiments, the mitotic inhibitor comprises ixabepilone, paclitaxel or eribulin. In some embodiments, the CDK inhibitor comprises palbociclib, abemaciclib or ribociclib. In some embodiments, the antibody therapy comprises APX005M, avelumab, bavituximab, bevacizumab, cixutumab, conatumumab, durvalumab, denosumab, dalotuzumab, ficlatuzumab, figitumumab, fresolimumab, Hu3S193, ipilimumab, MN-14, mapatumuzab, matuzumab, MEDI4736, necitumumab, nivolumab, nimotuzumab, nofetumomab, olaratumab, onartuzumab, pembrolizumab, panitumumab, pertuzumab, racotumomab, ramucirumab, rovalpituzumab, tucotuzumab, tremelimumab, trastuzumab, zalutumumab or a combination thereof. In some embodiments, the immune checkpoint inhibitor comprises a programmed cell death 1 (PD-1) inhibitor, a CD274 molecule (PD-L1) inhibitor or a cytotoxic T-lymphocyte associated protein 4 (CTLA-4) checkpoint inhibitor. In some embodiments, the immune checkpoint inhibitor comprises atezolizumab, durvalumab, ipilimumab, tremelimumab or indiximod.

In some embodiments of the methods of the disclosure, a subject is diagnosed as IL-1 positive based on SNPs at each of the rs17561 polymorphic locus, the rs16944 polymorphic locus, the rs1143634 polymorphic locus, the rs1143623 polymorphic locus and the rs4848306 polymorphic locus. In some embodiments, a subject is diagnosed as IL-1 positive if the subject has an IL-1 genotype pattern that is the same as any of: (i) T/T or T/G at rs17561, C/C, T/T, C/T or T/C at rs4848306, G/G at rs1143623, C/C at rs16944 and T/T or T/C at rs1143634; (ii) G/G at rs17561, C/C, T/T, C/T or T/C at rs4848306, G/G at rs1143623, C/C at rs16944 and C/C, T/T, C/T or T/C at rs1143634; (iii) G/G, T/T, G/T or T/G at rs17561, C/C, T/T, C/T or T/C at rs4848306, G/G at rs1143623, C/C at rs16944 and C/C at rs1143634; (iv) T/T or T/G at rs17561, C/C or C/T at rs4848306, G/G at rs1143623, C/T at rs16944 and T/T or T/C at rs1143634; (v) G/G at rs17561, C/C or C/T at rs4848306, G/G at rs1143623, C/T at rs16944 and C/C, T/T, C/T or T/C at rs1143634; (vi) G/G, T/T, G/T or T/G at rs17561, C/C or C/T at rs4848306, G/G at rs1143623, C/T at rs16944 and C/C at rs1143634; (vii) T/T or T/G at rs17561, C/C at rs4848306, C/G at rs1143623, C/T at rs16944 and T/T or T/C at rs1143634; (viii) G/G at rs17561, C/C at rs4848306, C/G at rs1143623, C/T at rs16944 and C/C, T/T, C/T or T/C at rs1143634; (ix) G/G, T/T, G/T or T/G at rs17561, C/C at rs4848306, C/G at rs1143623, C/T at rs16944 and C/C at rs1143634; (x) T/T or T/G at rs17561, C/C at rs4848306, G/G at rs1143623, T/T at rs16944 and T/T or T/C at rs1143634; (xi) G/G at rs17561, C/C at rs4848306, G/G at rs1143623, T/T at rs16944 and C/C, T/T, C/T or T/C at rs1143634; or (xii) G/G, T/T, G/T or T/G at rs17561, C/C at rs4848306, G/G at rs1143623, T/T at rs16944 and C/C at rs1143634.

In some embodiments of the methods of the disclosure, a subject is diagnosed as IL-1 positive based on SNPs at each of the rs17561 polymorphic locus, the rs16944 polymorphic locus and the rs1143634 polymorphic locus. In some embodiments, a subject is diagnosed as IL-1 positive if the subject has an IL-1 genotype pattern that is the same as any of: (xiii) T/T or T/G at rs17561, C/C at rs16944 and T/T/ or T/C at rs1143634; (xiv) G/G at rs17561, C/C at rs16944 and C/C, T/T, C/T or T/C at rs1143634; (xv) G/G, T/T, G/T or T/G at rs17561, C/C at rs16944 and C/C at rs1143634; and (xvi) T/T or T/G at rs17561, C/T at rs16944 and T/T or T/C at rs1143634.

In some embodiments of the methods of the disclosure, a subject is diagnosed as IL-1 positive based on SNPs at each of the rs16944 polymorphic locus, the rs1143623 polymorphic locus and the rs4848306 polymorphic locus. In some embodiments, a subject is diagnosed as IL-1 positive if the subject has an IL-1 genotype pattern that is the same as any of: (xvii) C/C, T/T, C/T or T/C at rs4848306, G/G at rs1143623, C/C at rs16944; (xviii) C/C or C/T at rs4848306, G/G at rs1143623, C/T at rs16944; (xix) C/C at rs4848306, C/G at rs1143623, C/T at rs16944; and (xx) C/C at rs4848306, G/G at rs1143623, T/T at rs16944.

In some embodiments of the methods of the disclosure, a subject is diagnosed as IL-1 positive based on SNPs either at each of the rs17561 polymorphic locus, the rs16944 polymorphic locus and the rs1143634 polymorphic locus or SNPS at each of the rs17561 polymorphic locus, the rs16944 polymorphic locus, the rs1143634 polymorphic locus, the rs1143623 polymorphic locus and the rs4848306 polymorphic locus, and the IL-1 positive genotypes are the same as any one of the IL-1 positive genotypes listed in tables 1 and 2.

Any aspect or embodiment described herein can be combined with any other aspect or embodiment as disclosed herein. While the disclosure has been described in conjunction with the detailed description thereof, the foregoing description is intended to illustrate and not limit the scope of the disclosure, which is defined by the scope of the appended claims. Other aspects, advantages, and modifications are within the scope of the following claims.

The patent and scientific literature referred to herein establishes the knowledge that is available to those with skill in the art. All United States patents and published or unpublished United States patent applications cited herein are incorporated by reference. All published foreign patents and patent applications cited herein are hereby incorporated by reference. All other published references, documents, manuscripts and scientific literature cited herein are hereby incorporated by reference.

Other features and advantages of the invention will be apparent from the following detailed description and claims.

### BRIEF DESCRIPTION OF THE DRAWINGS

FIG. 1 shows the incidence of lung cancer in a Caucasian population analyzed with respect to smoking and IL-1 genotype. 4,232 individuals were prospectively monitored for incidence of lung cancer for a mean of 14.7 years.

FIG. 2 is a table showing the incidence of lung cancer in a population of 4,209 Caucasian individuals analyzed with respect to smoking and IL-1 genotype.

### DETAILED DESCRIPTION OF THE INVENTION

The present invention is based upon the discovery that inflammation, such as that caused by an overproduction of IL-1, is associated with an increased risk of developing cancers and further, that specific IL-1 genotype patterns stratify subjects into groups relating to their member's likelihood of over-producing IL-1. It is thus possible to specifically target subjects who have high levels of inflammation and who are at risk of developing, or who have developed a cancer such as lung, colorectal or breast cancer, for additional screening, treatment with IL-1 inhibitors and other therapies. Additional screening can reduce the risk of developing a cancer such as lung or colorectal cancer. Additional therapies for those with cancer who are IL-1 positive can more effectively treat an existing cancer, for example by inhibiting metastasis. Cancers that can be treated by the compositions and methods of the disclosure include lung, colorectal and breast cancer.

### Inflammation and Cancer

Inflammation is a protective response to any challenge to one's body from external pathogens, damaging exposures such as ultraviolet radiation, or cells in the body that have become damaged or express surface markers that alter or subvert the host clearance systems, as occurs in tumor cells. There is a clear link between inflammation and an increased risk of developing cancer and/ or the progression of many types of cancers. These cancers include, but are not limited to lung cancer, breast cancer, gastric cancer, prostate cancer, hematological cancers, pancreatic cancer, and glioblastoma.

Chronic inflammation accounts for approximately 25% of human cancers, and is a recognized etiologic factor in carcinogenesis. Patients with chronic inflammatory and oxyradical overload diseases are thus at higher risk of developing cancer.

Chronic inflammation causes tumor development via several mechanisms.

First, chronic inflammation can induce oncogenic mutation (Grivennikov et al, 2010; Ozbabacan et al, 2014). Without wishing to be bound by theory, inflammatory processes may induce DNA mutations in cells via oxidative/nitrosative stress. This condition occurs when the generation of free radicals and active intermediates in a system exceeds the system's ability to neutralize and eliminate these free radicals and active intermediates (Federico et al, 2007). Chronic inflammation, i.e. chronic inflammation caused by infectious agents, inflammatory diseases, and other factors, causes various types of damage to nucleic acids, proteins and tissue via generation of reactive oxygen species and reactive nitrogen species (ROS/RNS generation). Under inflammatory conditions, reactive oxygen species (ROS) and reactive nitrogen species (RNS) are generated from inflammatory and epithelial cells (Ohnishi et al, 2003). When persistent infection induces chronic inflammation, products generated by leukocytes and other cells, including leukotrienes and reactive species of oxygen and nitrogen, can also contribute to DNA damage. Cancer occurs when the DNA of cells mutates in such a way that the cells escape normal controls on excessive growth and proliferation. Thus, the longer the inflammation persists, the higher the likelihood of cellular damage, and the higher the risk of cancer.

Second, tissue injury under chronic inflammation activates progenitor/stem cells for regeneration. In these cells, ROS/RNS from inflammation can cause multiple mutations, which may generate mutant stem cells and cancer stem cells, leading to carcinogenesis (Ohnishi et al, 2013). Inflammatory cells also release prostaglandins produced by the action of enzyme cyclooxygenase-2 (COX-2), which intensifies the inflammation and has an impact on various carcinogenic routes (Fernandes et al, 2015).

Third, inhibition or elimination of cell death and/ or repair programs also occurs in chronically inflamed tissues, resulting in DNA replication and the proliferation of cells that have lost normal growth control. Normal inflammation is self-limiting, because the production of anti-inflammatory cytokines follows the pro-inflammatory cytokines closely. However, chronic inflammation appears to be due to persistence of the initiating factors, a failure of mechanisms required for resolving the inflammatory response, or increased IL-1β levels resulting from dysfunctional control of IL-1 production, processing, and release.

Fourth, there are specific cellular signaling pathways linking inflammation and cancer. One such pathway is the hypoxia inducible factor 1 alpha (HIF-1α) signaling pathway. In one experiment, the HIF-1 signaling pathway was stimulated by IL-1β in A549 cells. The proinflammatory cytokine IL-1β up-regulates HIF-1α protein and activates the HIF-1-responsive gene vascular endothelial growth factor (VEGF), which is pivotal in metastasis via a pathway dependent on nuclear factor kappaB (NFκB). IL-1 mediated NFκB-dependent cyclooxygenases-2 (COX-2) expression serves as a positive effector for HIF-1α induction. Thus, IL-1β up-regulates functional HIF-1α protein through a classical inflammatory signaling pathway involving NFkB and COX-2, leading to up-regulation of VEGF, a potent angiogenic factor required for tumor growth and metastasis (Jung et al, 2003). In this manner, a pro-inflammatory signal (IL-1-NFκB-COX-2) is translated into an oncogenic signal (VEGF stimulation) through HIF-1α up-regulation and VEGF secretion.

In addition, IL-1β has been shown to act through the COX2-HIF1α pathway to repress the expression of microRNA-101 (miR-101), a microRNA with an established role in tumor suppression. In one study, IL-1β was dramatically elevated in the serum of patients with non-small cell lung cancer (NSCLC), which comprises 80% to 85% of all lung cancers. IL-1β promoted the proliferation and migration of NSCLC cells. Subsequent repression of mir-101 represents an important mechanism of its tumor-promoting activity (Wang et al, 2014a).

Alternatively, or in addition, several pro-inflammatory gene products have been identified that mediate a critical role in suppression of apoptosis, proliferation, angiogenesis, invasion, and metastasis. Among these gene products are TNF and members of its superfamily, IL-1α, IL-1β, IL-6, IL-8, IL-18, chemokines, matrix metallopeptidase 9 (MMP-9), vascular endothelial growth factor A (VEGF), prostaglandin-endoperoxide synthase 2 (COX-2), and arachidonate 5-lipoxygenase (5-LOX). The expression of these genes is mainly regulated by the transcription factor NF-κB, which is constitutively active in most tumors and is induced by carcinogens (such as cigarette smoke) (Aggarwal et al, 2006). For example, cigarette smoking and inhaled particulate matter can stimulate the airway or lung epithelium, thereby activating inflammasomes that induce the production of IL-1β and IL-18. Both IL-1β and IL-18 promote the epithelial to mesenchymal transition (EMT) and secretion of pro-inflammatory cytokines such as vascular endothelial growth factor A (VEGF), C-X-C motif chemokine ligand 2 (CXCL2) and hepatocyte growth factor (HGF). Proinflammatory cytokines affect the tumor microenvironment and promote lung cancer progression. Proinflammatory cytokines implicated in carcinogenesis include IL-1, IL-6, IL-15 and colony stimulating factors (CSF) (Hussain and Harris, 2007). The NLR family pyrin domain containing 3 (NLRP3) inflammasome promotes lung cancer by inhibiting natural killer cells (NKs). In contrast, absent in melanoma 2 (AIM2) suppresses lung cancer progression by activating NKs (He et al, 2018).

Without wishing to be bound by theory, it is likely that the processes by which chronic inflammation drives oncogenesis are through “landscaper” defects rather than a germ line genetic “gatekeeper” or “caretaker” defects (Kinzler and Vogelstein, 1998). Landscaper defects, as applied to chronic ulcerative colitis, for example, reflect factors that cause an abnormal microenvironment due to inflammation, and those factors in the microenvironment increase the risk of neoplastic transformation. Indeed, as stated above, many factors associated with chronic inflammation appear to increase genomic damage and cellular proliferation, which favor malignant transformation of pancreatic cells including various cytokines, reactive oxygen species, and mediators of the inflammatory pathway (e.g., NF—KB and cyclooxygenase-2), which increase cell cycling, cause loss of tumor suppressor function, and stimulate oncogene expression that may lead to pancreatic malignancy (Farrow and Evers, 2002).

Inflammation usually accompanies tumor development, and contributes to tumor-mediated angiogenesis. The role of IL-1β is also evident in these processes. Without wishing to be bound by theory, this may be due to IL-1β being secreted into the tumor microenvironment, thus activating cells in the tumor's stroma, including the malignant cells (Voronov et al, 2003). These findings agree with previous observations that tumor cells engineered to secrete IL-1 resulted in a more severe and invasive progression pattern compared with that of mock-transfected cells (Apte et al, 2000). Inflammation also contributes to tumor-mediated angiogenesis through IL-1β upregulation of the expression of vascular endothelial cell growth factor (VEGF) and its receptors on endothelial cells (EC) (Berse et al., 1999) or aortic smooth muscle cells (Starvri et al, 1995; Maruyama et al, 1999; Nasu et al, 2006). In one example, VEGF secretion from these cells was significantly inhibited by addition of the interleukin 1 receptor antagonist IL1-Ra (Voronov et al, 2014). In addition, administering IL-1β to mice with a subcutaneous melanoma increased tumor size and pulmonary metastasis (Bani et al, 1991; Weinrich et al, 2003). IL-1 also stimulated the proliferation of endothelial cells (EC), adhesion molecule expression and production of cytokines and inflammatory molecules in vitro. While IL-1 does not directly activate EC migration, proliferation, and organization into blood vessel-like structures, it does activate infiltrating myeloid cells causing them to produce a cascade of cytokines/chemokines, which further activate tissue resident ECs to produce direct pro-angiogenic factors, such as VEGF (Carmi et al., 2009) (Voronov et al, 2014). This suggests that IL-1-related molecules may be deeply involved in the control of the angiogenic process (Dinarello, 1996; Liss et al, 2001; Nadini and Carraro, 2005).

Proinflammatory cytokines and inflammation also play an important role in the etiology of prostate cancer (Xu et al, 2014). High IL-6 and IL-1β levels are poor prognostic factors for overall survival in a multivariate analysis (P=0.011 and P=0.048, respectively). Pancreatic cancer patients with both a high IL-6 level and a high IL-1β level also had shortened overall and progression-free survival, a reduction in the tumor control rate, and required a high dose intensity of gemcitabine (GEM) compared with patients with low levels of both IL-6 and IL-1β (Mitsunaga et al, 2013).

In addition to the pathways and mechanisms described herein, inflammation has been linked to specific types of cancers. For example, colon carcinogenesis frequently arises in subjects with inflammatory bowel diseases such as, for example, chronic ulcerative colitis and Crohn's disease (Coussens et al, 2002). As a further example, IL-1β has multiple roles in melanoma. IL-1β increases the mRNA expression of genes for the production of cyclooxygenase-2 (COX-2), which is involved in the conversion of arachidonic acid (AA) to prostaglandin-E2 (PGE2). IL-1β can also be excreted from the cell, where it contributes to stem cell differentiation state modulation, angiogenesis, tumor invasiveness, tumor growth, chemokine activation to recruit pro-inflammatory cells, and other effects (Schneider et al, 2014; Li et al, 2012; Wang et al, 2012).

In colitis-associated cancer (CAC) the pro-tumorigenic function of IL-1β has mainly been attributed to increased IL-6 secretion by intestine-resident mononuclear phagocytes (MPs) (Wang et al, 2014b). However, IL-1β also increases the self-renewal of cancer stem cells (CSCs) in the human colon. IL-1β may act through zinc finger E-box binding homeobox 1 (Zeb1) to induce epithelial-mesenchymal transition (EMT) in colon cancer cells. IL-1β-induced spheres displayed an up-regulation of stemness factor genes (for example, BMI1 proto-oncogene, polycomb ring finger (Bmi1) and Nestin) and increased drug resistance, both hallmarks of CSCs. Furthermore, expression of the EMT activator Zeb1 was increased in IL-1β-induced spheres, indicating that a close association between EMT and IL-1β-induced CSC self-renewal (Li et al, 2012). Inflammasomal production of IL-1β thus may contribute to the ability of cancer stem cells to self-renew as well as to increase stem cell marker expression and invasive capacity (Li et al, 2012; Wang et al, 2012; Schneider et al, 2014).

Glioblastoma multiforme (GBM), a fast growing glioma, is an incurable highly malignant tumor of the brain with poor prognosis. IL-1 acts as a tumor promoting agent in malignant glioma (Tarassishin et al, 2014a; Basu et al, 2004; Thornton et al, 2006). IL-1 is the strongest inducer of pro-angiogenesis and pro-invasion factors such as VEGF and MMPs in human astrocytes and glioma cells. IL-1 also activates Stat3, a transcription factor crucial in glioma progression. For example, IL-1 activated glioblastoma conditioned media enhanced angiogenesis and neurotoxicity (Tarassishin et al, 2014b).

Further evidence of the link between inflammation and cancer can be seen with pancreatic cancer. Chronic pancreatitis (CP), independent of the underlying cause, over the course of a number of decades, markedly increases the risk for pancreatic adenocarcinoma. The risk is potentiated by known cofactors such as tobacco smoking and, likely, by common genetic factors that are yet to be identified (Whitcomb, 2004).

In a further example, in the microenvironment of the bone marrow, IL-1 produced by myeloma pre-cursor plasma cells stimulates stromal cells to release IL-6, which in turn promotes the survival and expansion of the pre-myeloma cells and contributes to cancer progression (Lust and Donaven, 1999). Of 47 patients who received Anakinra with dexamethasone, progression-free disease lasted over 3 years and in 8 patients over 4 years (Lust et al, 2009). Compared to historical experience, these findings indicate a significant failure to progress to active disease. This poor response to treatment may be because both IL-1α and IL-1β contribute to tumor angiogenesis and invasiveness, which are key processes in the progression of tumors.

IL-1α-mediated systemic inflammation can also be a debilitating aspect of cancer. Many tumors produce IL-1α, which promotes angiogenesis and tumor growth (Krelin Y, et al, 2007). Unlike precursors of IL-1β, the IL-1α precursor is fully active. Neutralization of local IL-1α reduces the infiltration of tumor-associated macrophages and myeloid-derived suppressor cells, which contribute to the immunosuppression of cancer mediated by inflammation (Balkwill and Mantovani, 2012; Dinarello, 2014). Further, the expression of IL-1α was significantly associated with tumor size, FIGO histology grade, lymph node metastasis, stromal invasion, and tumor differentiation in human cervical cancer. IL-1α and IL-6 play crucial roles in cancer initiation, development, and metastasis (Liu et al, 2013). For example, IL-1α plays an important role in the development, invasion, and metastasis of cancer. IL-1α expression is elevated in a variety of cancers such as breast cancer (Kurtzman et al, 1999), pancreatic cancer, (Tang et al, 2005) and head and neck cancer (Wolf et al, 2001). Moreover, the enhanced expression of IL-1α, and IL-6, has been correlated with poorer prognosis of cancer (Song et al, 2016).

In summary, individuals with elevated IL-1 driven systemic inflammation not only have a greater risk for developing cancers or metastasis, but also frequently have a poor response to treatment.

### Cytokine Polymorphisms and Cancer

The instant invention is based on the finding that specific IL-1 genotype patterns identify subjects, referred to herein as IL-1 positive, who produce higher IL-1β protein levels when cells in local tissues are activated, and thereby drive higher innate immune responses compared to subjects who do not have these IL-1 positive genotype patterns. IL-1 genotype testing can thus be used to identify subjects at risk for certain cancers. These subjects can be subjects who are already at risk, or subjects who are not in populations traditionally thought of as at risk for certain cancers. The IL-1 genotype testing methods of the instant disclosure can be used to guide the intensity of screening, monitoring for early detection, and to guide specific interventions. The IL-1 genotype testing of the instant disclosure can also be used to identify subjects who, due to high familial risk, environmental risk or medical history, are more likely to benefit from early testing and/ or drug therapy to prevent development of certain cancers.

Certain cytokine single nucleotide polymorphisms (SNPs) have been associated with the occurrence of certain cancers. For example, increased gastric cancer risk is associated to IL1B-511, IL1RN variable number tandem repeat (VNTR), and TNFA-308, despite the heterogeneous findings across previous meta-analyses (Peleteiro et al, 2010). Further, the IL1β-511T polymorphism correlates with increased risk of developing gastric cancer in the global population (OR of 1.23, 95% CI 1.09-1.37, P=0.0002). In a further example, the analysis of the population stratified for Caucasian and Asian ethnicities showed that the IL1β-511T polymorphism correlates with a statistically significant increased risk of gastric cancer in the Caucasian (OR of 1.56, 95% CI 1.32-1.84, P<0.00001), but not in the Asian population (Vincenzi et al, 2008).

For non-small cell lung cancer (NSCLC), Landvik et al. (2009) reported an association between IL-1 genotype and NSCLC. Landvik et al. selected 6 SNPs from the promoter region of the IL1B gene: −3737 C>T (rs4848306), −1464 G>C (rs1143623), −511 C>T (rs16944), and −31 T>C (rs1143627), +3954 C>T (rs1143634), and −3893 G>A (rs12621220). Two of the SNPs, −1464 (rs1143623) and −3893 (rs12621220), were individually associated with increased risk of NSCLC. Landvik et al. then removed −3737 C>T (rs4848306) and +3954 C>T (rs1143634) and reported that one haplotype, GGCT, was significantly associated with non-NSCLC. Accordingly, Landvik et al. constructed the 4 SNP “high risk haplotype”, GGCT, and compared it to a “protective haplotype”, ACTC, relative to luciferase activity in the promoter-reporter construct. The GGCT haplotype produced significantly higher luciferase activity than did the ACTC haplotype. However, since the −3893 G>A (rs12621220) SNP used by Landvik et al. to generate their four SNP haplotype is non-functional with respect to transcriptional activity, all associations reported by Landvik and involving −3893 G>A (rs12621220), such as higher mRNA levels with the haplotype (GGCT), are in fact due to the effect of only three SNPs, −1464 G>C (rs1143623), −511 C>T (rs16944), and −31 T>C (rs1143627).

For colorectal cancer, Sanabria-Salas and colleagues (2017) reported a specific IL1B haplotype (CGTC) was strongly associated with colorectal cancer and was carried in close to 50% of subjects of African ancestry in the Columbian population studied. The IL-1B CGTC haplotype of interest to Sanabria-Salas included −3737 C>T (rs4848306), −1464 G>C (rs1143623), −511 C>T (rs16944), and −31 T>C (rs1143627), what is sometimes referred to as the B4 haplotype (Rogus et al., 2008). However, the Sanabria-Salas approach to defining whether or not a subject carries the IL-1B CGTC haplotype is flawed. It depends on a mathematical iterative process that starts with an un-admixed set of assumptions on distributions of the SNPs in the population. Information in the chromosomes of admixed subjects is inferred from local ancestry, which works if one starts out with a specific population group. However in North America, South America and much of Europe there is substantial admixture so there is no practical way to set a general assumption about the distribution of certain haplotypes, or to force a more narrow assumption because one must assume a very specific admixture. Unlike Sanabria-Salas et al. (2017), the methods of the instant invention are able to unambiguously identify the IL1B promoter haplotypes and avoid mathematical estimates that are highly prone to error given the broad range of carriage for the certain haplotypes across different racial groups.

Unlike other combinations of SNPs used to characterize IL-1 genotype, some embodiments of the instant disclosure stratify IL-1 haplotype pairs into IL-1 positive and IL-1 negative genotype patterns based on single nucleotide polymorphisms (SNPs) at 5 specific locations in the IL-1 locus. Specifically, the loci analyzed in the instant invention are rs16944, rs1143623, rs4848306, rs17561 and rs1143634 loci. Three of these loci, rs4848306, rs1143623 and rs16944, are functional SNPs in the IL-1B promoter/enhancer region.

The combinations of 3 and 5 polymorphic loci, and the haplotype pairs identified as IL-1 positive and IL-1 negative by the instant disclosure, provide superior methods of stratifying populations according to IL-1 genotype for the reasons outline below.

First, the disclosure provides methods to unambiguously identify a subject as IL-1 positive or IL-1 negative using haplotype pairs determined from a survey of naturally occurring haplotypes. The methods of the instant disclosure are able to determine whether a subject is IL-1 positive or IL-1 negative without recourse to statistical models that may not be applicable to all populations.

Second, the 5 SNP-based haplotype pairs of the instant disclosure have been analyzed relative to actual tissue fluid levels of IL-1β protein for more than 900 subjects carrying all of the 10 possible haplotype pairs. Additional populations have been analyzed for specific diseases. This allows the 5 SNP haplotype pairs of the instant disclosure to identify a subject's specific IL-1 haplotype pair, and define that subject as one who will produce high or lower levels of IL-13 when challenged. For example, the inventors have identified 3 haplotype pairs that are predictably high producers of IL-1 beta and 3 pairs that are predictably lower producers of IL-1β and 4 pairs that are somewhere in the middle. The high IL-1 producing haplotype pairs chronically produce approximately 30% higher tissue levels than the 3 lower producers.

Third, haplotype context is required for the different functional SNPs working together to regulate transcription of the IL1B gene in response to complex activation of transcription. This occurs if the functional SNPs have different activities depending on the specific context of the functional SNPs within the pattern. Transcription factors binding to one or more SNPs in the pattern bring together 3 dimensional nucleic acid structures to influence initiation of transcription and define the transcription rate.

Contrary to the routine mathematical projections of what haplotypes a subject may have at a specific genetic locus, the haplotypes of the instant disclosure are unambiguously determined from the composite genotype of the subject. Routine mathematical projections used in genotyping are based upon certain assumptions about the general population that may be influenced in the individual due to ancestry of the population from which they come. That means that for almost any place in North America, South America, and Europe, the admixture must be considered and therefore modifies the accuracy of the projection.

The ability to unambiguously define the two haplotypes carried by a specific subject provides greater precision in identifying which two haplotypes are carried by that subject. That capability exists because out of 8 possible haplotypes from 3 functional SNPs assayed, only 4 of the 8 are actually observed in nature across all major racial populations. However, these haplotypes are observed in different frequencies in different populations. For example one haplotype, termed B4 (Rogus et al., 2008), accounts for 6% of Caucasian haplotypes in the IL1 promoter, while the B4 haplotype accounts for 46% of haplotypes carried by subjects of African ancestry. Once the functional haplotypes have been unambiguously determined for a subject, the other two SNPs add further information about the biologic activity of the subject's IL-1 transcription rates when cells are activated. That provides, in some racial populations, a substantially different assessment of the subject's IL-1 biologic activity than one may derive from nonfunctional patterns that may be generated using standard mathematical formula.

Third, the inventors have determined that non-functional SNPs, rs17561 and rs1143634 are also associated with inflammatory biomarkers. In Caucasian populations, carriage of both minor alleles at rs17561 and rs1143634 is found in only approximately 35% of the population. However, the minor alleles are found in 84% of the pro-inflammatory haplotype pairs B1/B3, B3/B3, B2/B3, and B3/B4 identified in Rogus et al. (2008). Adding genotype information from rs17561 and rs1143634 to genotyping at rs4848306, rs1143623 and rs16944, and classifying the 5 SNP haplotype pairs of the instant invention, as shown in Table 1 below allows, for the first time, for the successful stratification of IL-1 haplotype pairs that are common in populations beyond those that are predominantly Caucasian, such as African-American populations.

Fourth, the 5 SNP haplotype patterns of the instant disclosure account for differences in ancestry to a greater degree than previous studies. The set of patterns that include 5 SNPs of the instant disclosure represent additional ancestry context that goes beyond the IL-1 beta haplotype. As a result, when the 5 SNPs of the instant disclosure were used to assess large databases, there were clear situations where subjects of African or Chinese ancestries have very different frequencies of 5 SNP patterns based on ancestry factors that go beyond the IL-1β haplotypes.

For example there are 3 extended patterns for IL-1β promoter haplotype pairs that combine the B1 and B3 IL-1B haplotypes described by Rogus et al. (2008). Those 3 extended patterns have substantially different frequencies for Caucasians and African-Americans. One of the 3, for example as a B1 B3 haplotype pair, is carried by 14% of Caucasians but 2% of African-Americans. One of the other two B1 B3 extended patterns is carried by 6% of African-Americans but only 1.2% of Caucasians.

The net result for subjects of African ancestry is that the approach described by Sanabria-Salas (2017) to determine risk of colorectal cancer will result in 12% false positive results compared to the methods of the instant disclosure. In addition, with one of the IL-1β genotype patterns of the instant invention, 12% of subjects who were tested by the Sanabria-Salas approach would be classified as positive for high risk for NSCLC, but the methods the instant invention would classify these same subjects as IL-1 gene test negative.

This same problem applies to prior studies such as Rogus et al. (2008) which was limited to a Caucasian population. The 5 SNP test of the instant invention provides more refined information about how IL-1 haplotype pairs translate into higher or lower IL-1β production across all major racial populations. If one used the Rogus et al. (2008) 3 SNP patterns on subjects with an African ancestry several patterns would produce false negative results compared to the 5-SNP patterns. This example would result in approximately 18% of subjects of African ancestry receiving a false negative IL-1 gene test if the Rogus 3 SNP test were used instead of the 5 SNP test of the instant invention.

The methods of the Landvik studies also have a high probability of producing false negatives. The Landvik studies require that the ordinarily skilled artisan determine the probability of a specific subject carrying a high risk IL-1 haplotype (GGCT). That haplotype will be paired with one of the 4 haplotypes in the IL-1B promoter region that can be identified by the instant disclosure. Since the gene expression is a function of how the pairs of haplotypes behave when inherited together, this means a percentage of subjects tested based on the Landvik report will be identified as false negatives for a high risk haplotype.

Given this superior ability to stratify diverse populations of subjects, the claimed 5 SNP haplotype pattern of the instant disclosure provide, for the first time, for the identification of subjects at a higher risk of lung or colorectal cancer, or undergoing breast cancer metastasis, and targeting appropriate treatments and/ or testing regimens to these subjects. The IL-1 genotype can be used to predict the response of a subject to anti-IL-1 therapy.

### Stratification by IL-1 Genotype

Subjects can be stratified into one of two IL-1 genotype patterns, i.e., positive or negative, based upon their complex IL-1 genotype for three or five single nucleotide polymorphisms (SNPs) in the IL-1 locus. IL-1 positive and IL-1 negative genotypes of the disclosure are listed in Table 1, Table 2 and Table 3 below.

TABLE 1

| | | | | | IL-1 |
|---|---|---|---|---|---|
| rs17561 +4845 | rs4848306 −3737 | rs1143623 −1464 | rs16944 −511 | rs1143634 +3954 | Pattern |
| T/‡ | †/† | G/G | C/C | T/† | Positive |
| G/G | †/† | G/G | C/C | †/† | Positive |
| ‡/‡ | †/† | G/G | C/C | C/C | Positive |
| T/‡ | C/† | G/G | C/T | T/† | Positive |
| G/G | C/† | G/G | C/T | †/† | Positive |
| ‡/‡ | C/† | G/G | C/T | C/C | Positive |
| T/‡ | C/C | C/G | C/T | T/† | Positive |
| G/G | C/C | C/G | C/T | †/† | Positive |
| ‡/‡ | C/C | C/G | C/T | C/C | Positive |
| T/‡ | C/T | C/G | C/T | T/† | Negative |
| G/G | C/T | C/G | C/T | †/† | Negative |
| ‡/‡ | C/T | C/G | C/T | C/C | Negative |
| T/‡ | C/C | G/G | T/T | T/† | Positive |
| G/G | C/C | G/G | T/T | †/† | Positive |
| ‡/‡ | C/C | G/G | T/T | C/C | Positive |
| T/‡ | C/C | C/* | T/T | T/† | Negative |
| G/G | C/C | C/* | T/T | †/† | Negative |
| ‡/‡ | C/C | C/* | T/T | C/C | Negative |

TABLE 2

| | | | IL-1 |
|---|---|---|---|
| rs17561 +4845 | rs16944 −511 | rs1143634 +3954 | Pattern |
| T/‡ | C/C | T/† | Positive |
| G/G | C/C | †/† | Positive |
| ‡/‡ | C/C | C/C | Positive |
| T/‡ | C/T | T/† | Positive |
| G/G | C/T | †/† | Negative |
| ‡/‡ | C/T | C/C | Negative |
| T/‡ | T/T | T/† | Negative |
| G/G | T/T | †/† | Negative |
| ‡/‡ | T/T | C/C | Negative |

TABLE 3

| | | | IL-1 |
|---|---|---|---|
| rs4848306 −3737 | rs1143623 −1464 | rs16944 −511 | Pattern |
| †/† | G/G | C/C | Positive |
| C/† | G/G | C/T | Positive |
| C/C | C/G | C/T | Positive |
| C/T | C/G | C/T | Negative |
| C/C | G/G | T/T | Positive |
| C/C | C/* | T/T | Negative |

In Tables 1-3, “*” is G or C; “t” is C or T; and “$” is G or T.

A subject having an uncommon complex IL-1 genotype not exemplified in Tables 1-3 is considered herein as having an IL-1 genotype pattern of “Negative”.

A subject may be stratified into an IL-1 genotype pattern by the SNP loci listed in Tables 1-3 and/ or SNP loci in linkage disequilibrium (LD), e.g., 80% LD, with the SNP loci listed in Tables 1-3.

A subject of certain racial/ethnic groups may be stratified into an IL-1 genotype pattern based upon five SNP loci listed in Table 1. Other racial/ethnic groups may require three SNP loci (as in Table 2 or Table 3) to be stratified into an IL-1 genotype pattern. Differences in the frequencies or even the absence of a specific SNP in certain racial/ethnic groups may require the inclusion of additional informative SNPs. For example, the three SNPs disclosed in Table 2 are able to stratify Caucasian populations, but may fail to accurately stratify Asian populations.

Accordingly, the disclosure provides methods of diagnosing a subject as being IL-1 positive or negative based on IL-1 positive or negative genotype patterns.

The methods comprise obtaining information regarding the subject's single nucleotide polymorphism (SNP) alleles for: (i) each of the rs17561 polymorphic locus, the rs16944 polymorphic locus and the rs1143634 polymorphic locus; (ii) each of the rs16944 polymorphic locus, the rs1143623 polymorphic locus and the rs4848306 polymorphic locus; or (iii) each of the rs17561 polymorphic locus, the rs16944 polymorphic locus the rs1143634 polymorphic locus, the rs1143623 polymorphic locus and the rs4848306 polymorphic locus.

In those embodiments comprising the SNPs at rs17561, rs16944 and rs1143634, a subject can be diagnosed as IL-1 positive if the subject has an IL-1 genotype pattern that is the same as any of: T/T or T/G at rs17561, C/C at rs16944 and T/T/ or T/C at rs1143634; G/G at rs17561, C/C at rs16944 and C/C, T/T, C/T or T/C at rs1143634; G/G, T/T, G/T or T/G at rs17561, C/C at rs16944 and C/C at rs1143634; and T/T or T/G at rs17561, C/T at rs16944 and T/T or T/C at rs1143634.

In those embodiments comprising the SNPs at rs16944, rs1143623 and rs4848306, a subject can be diagnosed as IL-1 positive if the subject has an IL-1 genotype pattern that is the same as any of: C/C, T/T, C/T or T/C at rs4848306, G/G at rs1143623, C/C at rs16944; C/C or C/T at rs4848306, G/G at rs1143623, C/T at rs16944; C/C at rs4848306, C/G at rs1143623, C/T at rs16944; or C/C at rs4848306, G/G at rs1143623, T/T at rs16944.

In those embodiments comprising the SNPs at rs17561, rs16944, rs1143634, rs1143623 and rs4848306, a subject can be diagnosed as IL-1 positive if the subject has an IL-1 genotype pattern that is the same as any of: (i) T/T or T/G at rs17561, C/C, T/T, C/T or T/C at rs4848306, G/G at rs1143623, C/C at rs16944 and T/T or T/C at rs1143634; (ii) G/G at rs17561, C/C, T/T, C/T or T/C at rs4848306, G/G at rs1143623, C/C at rs16944 and C/C, T/T, C/T or T/C at rs1143634; (iii) G/G, T/T, G/T or T/G at rs17561, C/C, T/T, C/T or T/C at rs4848306, G/G at rs1143623, C/C at rs16944 and C/C at rs1143634; (iv) T/T or T/G at rs17561, C/C or C/T at rs4848306, G/G at rs1143623, C/T at rs16944 and T/T or T/C at rs1143634; (v) G/G at rs17561, C/C or C/T at rs4848306, G/G at rs1143623, C/T at rs16944 and C/C, T/T, C/T or T/C at rs1143634; (vi) G/G, T/T, G/T or T/G at rs17561, C/C or C/T at rs4848306, G/G at rs1143623, C/T at rs16944 and C/C at rs1143634; (vii) T/T or T/G at rs17561, C/C at rs4848306, C/G at rs1143623, C/T at rs16944 and T/T or T/C at rs1143634; (viii) G/G at rs17561, C/C at rs4848306, C/G at rs1143623, C/T at rs16944 and C/C, T/T, C/T or T/C at rs1143634; (ix) G/G, T/T, G/T or T/G at rs117561, C/C at rs4848306, C/G at rs1143623, C/T at rs16944 and C/C at rs1143634; (x) T/T or T/G at rs17561, C/C at rs4848306, G/G at rs1143623, T/T at rs16944 and T/T or T/C at rs1143634; (xi) G/G at rs117561, C/C at rs4848306, G/G at rs1143623, T/T at rs16944 and C/C, T/T, C/T or T/C at rs1143634; or (xii) G/G, T/T, G/T or T/G at rs17561, C/C at rs4848306, G/G at rs1143623, T/T at rs16944 and C/C at rs1143634.

A subject at risk for lung cancer, colorectal cancer or metastatic breast cancer, or in need of treatment for lung, colorectal or breast cancer, will provide or has provided a biological sample comprising a nucleic acid. Single nucleotide polymorphism (SNP) alleles in the isolated nucleic acid for each of the, at least 3, or 5 polymorphic loci identified in Tables 1-3, or polymorphic loci in linkage disequilibrium to the polymorphic loci identified in Tables 1-3 will be detected by any method known in the art and a composite IL-1 genotype will be determined. From the determined composite IL-1 genotype, a positive or negative IL-1 genotype pattern will be determined based on the information disclosed in Tables 1-3.

### Inflammation Inhibitors

The present invention allows for optimal treatment for a subject based upon his/her IL-1 genotype pattern. For subjects with high levels of inflammation, for example subjects diagnosed as IL-1 positive using the methods of the instant disclosure, this treatment can include an inflammation inhibitor to lower levels of inflammation. The inflammation inhibitor can be, for example, an IL-1 inhibitor, an IL-6 inhibitor, a GM-CSF inhibitor or a JAK/STAT inhibitor. Inflammation inhibitors reduce key leverage points of the biological cascades leading to inflammation, such as IL-1β and IL-6.

Due to the possibility of side effects and adverse events, the ability to predict which subjects that could derive a clinical benefit from IL-1 inhibitors from those that will not is critical for the success of this class of drugs. IL-1 inhibitors, such as IL-1β inhibitors, in general suppress the IL-1 mediated innate immune response and increase the risk of fatal infection. Thus, in IL-1 negative subjects without higher IL-1 driven levels of inflammation, treatment with an IL-1 inhibitor is more likely to result in immunosuppression and infection. In contrast, subjects with higher IL-1 driven inflammation are more likely to benefit from anti-IL-1 treatment, and less likely to experience suppression of innate immunity with the associated risk of infection.

IL-6 is acts as both a pro-inflammatory cytokine and an anti-inflammatory myokine, and is thought to stimulate inflammatory and auto-immune processes in many diseases. IL-6 acts downstream of IL-1 to mediate the inflammatory response.

Accordingly, the disclosure features methods for predicting the risk of and preventing lung and colorectal cancer in a human subject comprising diagnosing a subject as IL-1 positive using the SNP genotypes described herein, and optionally administering an IL-1 inhibitor if the subject is diagnosed as IL-1 positive to reduce inflammation and thereby reduce the risk of developing lung or colorectal cancer.

The disclosure features methods for reducing metastatic breast cancer comprising diagnosing a subject as IL-1 positive using the SNP genotypes described herein, and administering an IL-1 inhibitor if the subject is diagnosed as IL-1 positive to reduce inflammation and thereby reduce the risk of metastasis.

The disclosure also features methods for treating lung and colorectal cancer comprising diagnosing a subject as IL-1 positive using the SNP genotypes described herein, and administering an IL-1 inhibitor to the subject.

In some embodiments, if the subject has a positive IL-1 genotype pattern and one or more risk factors, biomarkers associated with lung cancer, or a diagnosis of lung cancer, the subject is administered an IL-1 inhibitor.

In some embodiments, if the subject has a positive IL-1 genotype pattern and one or more risk factors, biomarkers associated with colorectal cancer, or a diagnosis of colorectal cancer, the subject is administered an IL-1 inhibitor.

In some embodiments, if the subject has a positive IL-1 genotype pattern and breast cancer, the subject is administered an IL-1 inhibitor.

IL-1 inhibitors of the disclosure can be administered to the subject in combination with one or more additional cancer therapies. The methods of the instant disclosure, including genotype testing and administration of IL-1 inhibitors, can be used in conjunction with any cancer therapy known in in the art.

The present invention, in view of the disclosures of Tables 1-3, allows a skilled artisan to identify: subjects likely to derive more benefit from an IL-1 inhibitor, such as an IL-1 α or IL-1β inhibitor; subjects with a positive IL-1 genotype pattern who may respond favorably to lower levels of an IL-1 inhibitor than subjects of a negative IL-1 genotype pattern; subjects who should be on an IL-1-1 inhibitor earlier than others because their genotype pattern is more aggressive; and subjects with an IL-1 dominant lung, colorectal or breast cancer subtype that may be predictably responsive to IL-1 inhibitors but not other agents which have different modes of action.

Modulators of IL-1 biological activity (e.g., IL-1α, IL-1β, or IL-1 receptor antagonist) or a protein encoded by a gene that is in linkage disequilibrium with an IL-1 gene, can comprise any type of compound, including a protein, peptide, peptidomimetic, lipid, small molecule, or nucleic acid. A modulator may be a botanical, or an extract of a botanical.

A modulator may indirectly act upon an IL-1 gene in that the modulator activates or represses a gene or protein that, in turn or ultimately, acts upon the IL-1 gene. As used herein, the term “ultimately” is meant that the modulator acts upon a first gene or protein and the first gene or protein directly acts upon the IL-1 gene or the first gene or protein acts upon a second gene or protein which directly (or indirectly) acts upon the IL-1 gene. Such indirect gene regulation is well known in the art. A modulator that acts upstream to the IL-1 gene is useful in the present invention. An example of a modulator that acts upstream of the IL-1 gene is Aldeyra's NS2 compound which traps excess free aldehydes, which are known to activate a number of intracellular inflammatory factors including NF-kB, a prominent protein in the inflammatory response. Another example of that acts upstream of the IL-1 gene is Ionis Pharmaceutical's IONIS-APO(a)-L_{Rx} and Arrowhead's ARC-LPA, which reduces Lp(a) levels that would be expected to activate arterial wall macrophages to produce IL-1β.

Alternately, a modulator may act downstream of the IL-1 gene by directly or indirectly affecting a gene or protein that operates in parallel to IL-1 in an inflammatory cascade.

An agonist can be a protein or derivative thereof having at least one bioactivity of the wild-type protein, e.g., receptor binding activity. An agonist can also be a compound that upregulates expression of a gene or which increases at least one bioactivity of a protein. An agonist can also be a compound which increases the interaction of a polypeptide with another molecule, e.g., a receptor.

An inhibitor (sometimes referred to as an antagonist) can be a compound which inhibits or decreases the interaction between a protein and another molecule, e.g., blocking the binding to receptor, blocking signal transduction, and preventing post-translation processing (e.g., IL-1 converting enzyme (ICE) inhibitor). The IL-1β converting enzymes, such as caspase 1, which are produced within inflammasomes to cleave the IL-1β pro peptide produce the mature cell-secreted IL-1β protein. An inhibitor can also be a compound that downregulates expression of a gene or which reduces the amount of a protein present. The inhibitor can be a dominant negative form of a polypeptide, e.g., a form of a polypeptide which is capable of interacting with a target. Inhibitors include nucleic acids (e.g., single (antisense) or double stranded (triplex) DNA or PNA and ribozymes), protein (e.g., antibodies) and small molecules that act to suppress or inhibit IL-1 transcription and/ or protein activity.

An anti-inflammatory drug refers to any agent or therapeutic regimen (including a pharmaceutical, biologic, nutraceutical, and botanical) that prevents or postpones the development of or alleviates a symptom of the particular disease, disorder, or condition that involved an inflammatory process in the subject. The drug can be a polypeptide, peptidomimetic, nucleic acid or other inorganic or organic molecule, a “small molecule,” vitamin, mineral, or other nutrient. The drug modulates the production of the active IL-1β or IL-1α polypeptides, or at least one activity of an IL-1 polypeptide, e.g., interaction with a receptor, by mimicking or potentiating (agonizing) or inhibiting (antagonizing) the effects of a naturally-occurring polypeptide. An anti-inflammatory drug also includes, but is not limited to, anti-cholesterol drugs (e.g., statins), diabetes mellitus drugs, drugs that treat acute syndromes of the heart and vascular system (e.g., a cardiovascular disease), and arthritis.

Non-limiting examples of anti-inflammatory agents that modulate or inhibit IL-1 biological activity useful in the present invention are listed in Table 4. These agents generally have a mode of action that includes modulation of IL-1 gene expression, modulation of inflammasomes, IL-1 receptor blocking agents, and agents that bind IL-1β or IL-1α to inhibit attachment to the active receptor. IL-1 blocking agents may also indirectly target IL-1 by blocking key activators of IL-1 gene expression.

TABLE 4

| | |
|---|---|
| ABT -981 | Gevokizumab |
| AC-701 | Givinostat |
| Ammonium trichloro-tellurate | Isunakinra |
| Anakinra | Rilonacept |
| Anakinra Biosimilar | RON-2315 |
| APX-002 | Sairei-To |
| Binimetinib | SER-140 |
| Can-04 | Tadekinig-alpha |
| Canakinumab | Xilonix |
| Diacerein | XL-130 |
| DLX-2681 | NUTRILITE ® IL1 Heart Health |
| | Nutrigenomic Dietary Supplement |
| DOM4-130-201 antibody | DOM4-130-202 antibody |
| Bermekimab | MABp1 antibody |

IL-1 inhibitors of the disclosure can inhibit IL-1β, IL-1α, or both IL-1β and IL-1α.

Exemplary IL-1β inhibitors include ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, Canakinumab, a DOM4-130-201 and DOM4-130-202 antibody. In some embodiments, the IL-1β inhibitor is Canakinumab or a derivative thereof.

Exemplary IL-1α inhibitors include Bermekimab, ABT-981, Isunakinra, AC-701, Sairei-To, Can-04, XL-130, a MABp1 antibody and Givinostat. In some embodiments, the IL-1a inhibitor is Bermekimab or a derivative thereof.

In some embodiments, the IL-1 inhibitor comprises an inflammasome modulator. In some embodiments, the inflammasome modulator can cross the blood brain barrier. In some embodiments, the inflammasome modulator comprises Diacerein, Sarei-To, Binimetinib, Can-04, Rilonacept, XL-130, Givinostat or Ammonium trichloro-tellurate.

In some embodiments, the inflammation inhibitor is an interleukin 6 (IL-6) inhibitor. IL-6 is a multifunctional cytokine. IL-6 inhibitors include inhibitors that target IL-6 and the interleukin 6 receptor (IL6R), for example antibodies, biologics or small molecules that bind to IL-6 or IL6R. Exemplary IL-6 inhibitors are shown in Table 5 below:

TABLE 5
IL-6 inhibitors

| | |
|---|---|
| Tocilizumab (Actemra) | Siltuximab (Sylvant) |
| Olokizumab (CPD6038) | Elsilimomab (BMS-945429) |
| Sirukimab (CNTO 136) | Levilimab (BCD-089) |
| ALX-0061 | Gerilimzumab (ARGX-109 |
| FE301 | FM101 |
| Sarilumab (Kevzara) | |

In some embodiments, the inflammation inhibitor is an inhibitor of an IL-1 driven inflammatory mediator. The IL-1 driven inflammatory response acts through, and in concert with, a number of additional factors (mediators), inhibitors of any one of which are envisaged as within the scope of the instant disclosure. These inflammatory mediators include the cytokines IL-6, IL-8, and IL-10, as well as granulocyte-macrophage colony-stimulating factor (GM-CSF) and members of the JAK/STAT signaling pathway.

In some embodiments, the inhibitor of an IL-1 driven inflammatory mediator is a GM-CSF or a Janus kinase/signal transducer and activator of transcription (JAK/STAT) inhibitor. In some embodiments, the JAK/STAT and/ or GM-CSF inhibitor is selected from the group disclosed in Table 6. In some embodiments, the JAK/STAT inhibitor is selected from the group consisting of baricitinib, upadacitinib and tofacitinib. Exemplary GM-CSF inhibitors include mavrilimumab, MOR103, lenzilumab, and MORAb-002. Exemplary GM-CSF and/or JAK/STAT inhibitors are shown in Table 6 below:

TABLE 6
GM-CSF and/or JAK/STAT inhibitors

| | |
|---|---|
| baricitinib | upadacitinib |
| tofacitinib | ruxolitinib |
| oclacitinib | peficitinib |
| fedratinib | cerdulatinib |
| gandotinib | lestaurtinib |
| pacritinib | abrocitinib |
| Cucurbitacin I | CHZ868 |
| mavrilimumab | MOR103 |
| Namilumab (MT203) | lenzilumab (KB003) |
| MORAb-002 | |

Any of the agents listed in Tables 4-6 may be used in the present invention. The subject may be administered one or more agents of Table 4-6 at a higher dose or at a lower dose (e.g., the dose of a single treatment and/ or a daily dose comprising one or more single treatments) depending on his/her IL-1 genotype pattern and status of one or more clinical indicators such as cancer risk factors or cancer diagnosis. Alternately, the subject may be not given the particular agent depending on his/her IL-1 genotype pattern and status of one or more clinical indicators, and instead may be administered a different agent.

Additionally, agents other than those listed in Tables 4-6 may be used in the present invention. For this, an alternate agent having a mode of action (MOA) similar to or identical to a drug listed in Tables 4-6 may be provided instead of or in addition to the agents listed in Tables 4-6. One skilled in the art is able to determine alternate agents that are useful in the present invention.

A subject may be administered one or more agents from Tables 4-6 or one or more alternate agents having a MOA similar to or identical to an agent listed in Tables 4-6 at the standard therapeutic dose. An agent may be given at a dose lower than the standard therapeutic dose, e.g., 99%, 95%, 90%, 85%, 80%, 75%, 70%, 60%, 50%, 40%, 30%, 20%, 15%, 10%, or 5%, and any percentage in between lower than the standard therapeutic dose. A agent may be given at a dose higher than the standard therapeutic dose, e.g., 5%, 10%, 15%, 20%, 30%, 40%, 50%, 60%, 70%, 75%, 80%, 85%, 90%, 95%, 100%, 125%, 150%, 175%, 200%, 300%, 400%, 500%, 600%, 700%, 800%, 900%, 1000%, 2000%, or more, and any percentage in between higher than the standard therapeutic dose. For example, if a standard therapeutic dose is 10 mg per day, a subject may be given 7 mg per day as a lower than standard therapeutic dose or 13 mg per day as a higher than standard therapeutic dose.

In some embodiments, for example in those embodiments where the subject is at risk of or diagnosed with lung cancer, the IL-1 inhibitor is formulated as an aerosol. Aerosols are can be inhaled into the lungs, and are thus able to target IL-1 inhibitors to inflamed lung tissues. In some embodiments, the aerosol is administered as a nasal spray.

In some embodiments, the IL-1β inhibitor is Canakinumab or a derivative thereof. In some embodiments, Canakinumab is administered to the subject at a dose of 25 mg to 300 mg. In some embodiments, the subject weighs less than 40 kg and the Canakinumab is administered to the subject at a dose of 2 mg/kg or 4 mg/kg. Alternatively, when the subject weighs more than 40 kg, the Canakinumab can be administered to the subject at a dose of 150 mg or 300 mg. Canakinumab can be administered every 2 weeks, every 4 weeks, every 6 weeks, every 8 weeks, every 10 weeks, every 3 months, every 5 months or every 6 months from the first administration. In some aspects, the Canakinumab is administered every 4 weeks from the first administration. In some aspects, Canakinumab is administered parenterally. Parenteral administration includes intravenous injection, intravenous infusion, intramuscularly, via intrapulmonary administration or subcutaneously.

In some embodiments, the IL-1α inhibitor is Bermekimab. In some aspects, the Bermekimab is administered at between 3 mg/kg to 20 mg/kg. In some aspects, the Bermekimab is administered at 7.5 mg/kg. Parenteral administration includes intravenous injection, intravenous infusion, intramuscularly, via intrapulmonary administration or subcutaneously. In some aspects, the Bermekimab is administered every week, every two weeks, every three weeks, every 4 weeks, every 5 weeks, every 6 weeks or every 8 weeks.

In some embodiments, administering the IL-1 inhibitor to a subject who is IL-1 positive reduces the risk of developing a cancer. For example, administering IL-1 inhibitors to subjects with one or more risk factors or biomarkers associated with lung or colorectal cancer can reduce the risk of developing lung or colorectal cancer.

In some embodiments, administering an IL-1 inhibitor to a subject who is IL-1 positive and has breast cancer reduces the risk of metastatic breast cancer in the subject.

In some embodiments, administering an IL-1 inhibitor to a subject who is IL-1 positive and who has cancer reduces a sign or a symptom of the cancer. The cancer can be lung, colorectal or breast cancer. Administering the IL-1 inhibitor can reduce a number of tumors of the cancer, reduce a size of a tumor of the cancer, reduce a growth rate of a tumor of the cancer, reduce early metaplastic changes in the cancer, reduce neo-angiogenesis, reduces tissue invasiveness by the cancer, reduces tissue invasion by the cancer through a basement membrane, reduce invasion of bone by the cancer, reduce metastasis of the cancer to distant organs, or a combination thereof.

In some embodiments, administering an IL-1 inhibitor to a subject who is IL-1 positive and who has cancer reduces a level of one or more biomarkers associated with the cancer. The cancer can be lung, colorectal or breast cancer. In those embodiments where subjects are IL-1 positive and have lung cancer, administering an IL-1 inhibitor can reduce the level of one or more biomarkers associated with lung cancer. In those embodiments where subjects are IL-1 positive and have colorectal cancer, administering an IL-1 inhibitor can reduce the level of one or more biomarkers associated with colorectal cancer. In those embodiments where subjects are IL-1 positive and have breast cancer, administering an IL-1 inhibitor can reduce the level of one or more biomarkers associated with breast cancer, for example biomarkers associated with breast cancer metastasis. All biomarkers associated with lung, colorectal and breast cancer are envisaged as within the scope of the instant disclosure. Exemplary biomarkers include, but are not limited to, angiogenic factors, a cancer associated proteins, RNAs, DNAs, micro-RNAs, exosomes, a circulating tumor cells, changes in metabolites or changes in DNA methylation status associated with cancers.

### Lung Cancer

The disclosure provides methods for reducing the risk of developing lung cancer in a subject who does not have lung cancer but is IL-1 positive.

The disclosure further provides methods of treating lung cancer in a subject comprising: determining whether the subject is IL-1 genotype positive or IL-1 genotype negative using the methods described herein, and administering an IL-1 inhibitor to the subject diagnosed as having an IL-1 positive genotype. In some embodiments, identifying a subject who has lung cancer comprises: (i) identifying a subject who has one or more risk factors or biomarkers of lung cancer; and (ii) testing the subject for lung cancer.

Lung cancer is a disease in which malignant cancer cells form in the tissues of the lung. Primary lung cancer starts in the lungs, from cells that are lung cells. Secondary lung cancer occurs when cancer cells travel from a cancer in another part of the body or metastasize to the lungs. There are two main subtypes of primary lung cancers: non-small lung cancers (NSCLC), and small cell lung cancers (SCLC). Lung cancers further comprise lung carcinoid tumors, mesotheliomas, Pancoast tumors, sarcomas and rare lung cancers such as adenoid cystic carcinomas and lymphomas.

In some embodiments, the lung cancer of the disclosure is a non-small cell lung cancer (NSCLC). NSCLC comprises about 80 to 85% of all lung cancers. NSCLC comprises multiple subtypes of cancers which arise from different types of lung cells. These subtypes of NSCLC comprise squamous cell carcinomas, adenocarcinomas and large cell carcinomas. Adenocarcinomas comprise about 40% of all lung cancers. Adenocarcinomas arise from glandular (i.e. secretory) cells in epithelial tissues. Squamous cell carcinomas, sometimes called epidermoid carcinomas, comprise about 25 to 30% of all lung cancers. Squamous cell carcinomas arise from squamous cells, which are thin, flat cells that line the surface of the lung. Large cell carcinomas, sometimes called undifferentiated carcinomas, comprise 10% to 15% of all lung cancers. Large cell carcinomas can appear in any part of the lung, and tend to grow and spread quickly. Large cell carcinomas can be particularly hard to treat because of this rapid growth. A subtype of large cell carcinoma, large cell neuroendocrine carcinoma, is a fast growing and highly aggressive cancer that resembles small-cell lung cancer (SCLC).

In some embodiments, the lung cancer of the disclosure is a small cell lung cancer (SCLC). SCLCs comprise about 10% to 15% of lung cancers. Small cell lung cancers comprise small cell carcinoma (sometimes called oat cell cancer) and combined small cell carcinoma. In some embodiments, SCLC is a neuroendocrine carcinoma that exhibits aggressive behavior, rapid growth, early spread to distant sites. SCLCs are frequently sensitive to chemotherapy and radiation.

In some embodiments, the lung cancer of the disclosure is a lung carcinoid tumor. Lung carcinoid tumors, sometimes called lung carcinoids, are a rare form of lung cancer. Lung carcinoid tumors tend to be slow growing. Lung carcinoid tumors arise from neuroendocrine cells. Neuroendocrine cells are hormone producing cells that make hormones such as adrenaline.

In some embodiments, the lung cancer of the disclosure is a Pancoast tumor. Pancoast tumors, sometimes called superior sulcus tumors, are tumors that are located at the apex of the lung. In some embodiments, a Pancoast tumor is a NSCLC. In some embodiments, a Pancoast tumor is a SCLC. In some embodiments, the Pancoast tumor is a carcinoma, for example a NSCLC squamous cell carcinoma. In some embodiments, the Pancoast tumor principally involves chest wall structures, for example the lymphatic system, the lower roots of the brachial plexus, the intercostal nerves, the stellate ganglion, sympathetic chain, adjacent ribs and/or vertebrae.

In some embodiments, the lung cancer of the disclosure is a sarcoma. Sarcomas are rare cancers that arise from mesenchymal cells, such as cells found in connective tissues. In some embodiments, the sarcoma is a metastatic sarcoma that has spread to the lungs from a primary tumor elsewhere in the body. In some embodiments, the sarcoma is a primary lung sarcoma. Primary lung sarcomas comprise ˜0.5% of lung cancers.

All types of lung cancers are envisaged as being treated by the compositions and methods of the disclosure.

### Lung Cancer Risk Factors

In some embodiments, the subject does not have a risk factor for lung cancer, or does not have a conventional risk factor acknowledged by the medical community. For example, smoking is a risk factor for lung cancer. However, current US Preventive Services Task Force (USPSTF) guidelines recommend computed tomography (CT) screening for lung cancer only in high risk smokers who are age 55-80, who quit less than 15 years previously, and have a smoking history that includes more than 30 pack years of smoking. The methods of the current disclosure, by identifying subjects who are IL-1 positive, is able to identify subjects outside of conventional “at-risk” populations such as the one identified by the USPSTF, and target these subjects for additional screening and other preventative measures.

Further, implementation of the USPSTF guidelines has proved expensive and challenging, as CT screening is expensive, can have relatively low acceptance among patients, and frequently yields false positives, which makes it a problematic initial screening tool. Thus, the methods of the instant disclosure, by identifying subjects who are IL-1 positive and at increased risk for lung cancer, are able to better identify subjects in need of additional screening or treatment.

In some embodiments, the subject has one or more risk factors for lung cancer. In those embodiments where the subject has one or more risk factors for lung cancer, diagnosing the subject as IL-1 positive using the methods of the instant disclosure can target the subject for additional screening, and optionally therapeutic intervention.

Chronic inflammation also adds to other risk factors, such as smoking, to increase relative risk for certain cancers. For example, T allele carriers of the IL1B rs1143634 polymorphism have a higher risk of lung cancer, especially among smokers. The attributable proportion due to interaction (AP) between IL1B rs1143634 genotypes and smoking was estimated to be 0.45 (95% CI 0.08-0.83, p=0.02). This measure was not equal to zero, suggesting the existence of an additive interaction (Kiyohara et al, 2010). Repetitive exposure to tobacco smoke promotes tumor development both in carcinogen-treated mice and in transgenic mice undergoing sporadic K-Ras activation in lung epithelial cells. Tumor promotion is due to induction of inflammation that results in enhanced pneumocyte proliferation and is abrogated by IKKb ablation in myeloid cells or inactivation of JNK1. Furthermore, induction of a low grade (subacute) inflammatory response contributes to the tumor-promoting activity of tobacco smoke, leading to the enhanced proliferation of both premalignant and malignant pulmonary epithelial cells in the formation of lung cancer (Takahashi et al, 2010).

Risk factors for the development of lung cancer include genetic risk factors, physiological risk factors such as a previous history of lung cancer, lung nodules or masses, or the presence of lung cancer biomarkers, and environmental risk factors such as exposure to radiation and carcinogens. Risk factors of the disclosure may contribute singly to an increased risk of developing lung cancer. Alternatively, risk factors of the disclosure may act additively or synergistically to drastically increase the risk of lung cancer.

In some embodiments of the methods of the disclosure, the one or more risk factors for lung cancer comprises smoking. Smoking is the leading risk factor for lung cancer. About 80% of all lung cancer deaths are thought to result from smoking. In men, the estimated cumulative risk of death by lung cancer by 75 years of age among smokers ranges from 14 to 28%. The association between smoking and SCLC is particularly strong. All forms of tobacco smoking, including cigars, pipes and cigarettes increase the risk of developing lung cancer. In some aspects, the smoking history comprises at least 30 pack years. In some aspects, the smoking history comprises less than 15 years since quitting. In some aspects, the smoking history comprises at least 30 pack years and less than 15 years since quitting.

In some embodiments of the methods of the disclosure, the one or more risk factors for lung cancer comprises exposure to second hand smoke. Even if the subject does not smoke tobacco, second hand smoke, sometimes called environmental tobacco smoke, increases the risk of developing lung cancer. Secondhand smoke is thought to cause more than 7,000 deaths per year from lung cancer.

In some embodiments of the methods of the disclosure, the one or more risk factors for lung cancer comprises exposure to asbestos. Asbestos is a set of naturally occurring silicate minerals which form long, thin crystals or fibers that can be released into the air and inhaled. Without wishing to be bound by theory, it is thought that asbestos fibers inhaled into the lungs cause genetic damage to cells that leads to a loss of controlled cell growth and the development of cancer. Asbestos can be found in many environments, which include, but are not limited to, mines, mills, textile factories, buildings with asbestos insulation or fire protection, and shipyards. Asbestos exposure can act synergistically with other risk factors, such as smoking, to greatly increase the risk of lung cancer.

In some embodiments of the methods of the disclosure, the one or more risk factors for lung cancer comprises exposure to radon. Radon is a naturally occurring radioactive gas that arises from the breakdown of uranium in soil and rocks. Breathing radon exposes the cells of the lungs to small amounts of radiation, which can cause genetic damage leading to cancer. Indoor environments, for example basements, can concentrate radon gas, increasing the level of exposure. According to the EPA, radon is the second leading cause of lung cancer.

In some embodiments of the methods of the disclosure, the one or more risk factors for lung cancer comprises exposure to diesel exhaust. Exposure to high levels diesel exhaust, such as in the workplace, has been tied to an increased risk of developing lung cancer. Diesel exhaust comprises a mixture of gases and particulates, which includes carcinogens such as sulfur oxides, polycyclic aromatic hydrocarbons, and trace metallic compounds. Diesel exhaust has been found to cause genetic changes in cells, which leads to cancer.

In some embodiments of the methods of the disclosure, the one or more risk factors for lung cancer comprises inhalation of carcinogens or carcinogenic chemicals. Carcinogens are agents that alter the genetic structure of cells, so that the cells escape controls on cell growth, multiply, and become malignant. Carcinogens can be single chemicals or agents (e.g., benzene), or mixtures of tens, hundreds or even thousands of chemicals, such as tobacco smoke or diesel exhaust. Exemplary carcinogens that increase the risk of lung cancer when inhaled include, but are not limited to, tobacco smoke, diesel exhaust and particulate air pollution. Exemplary carcinogenic chemicals that increase the risk of lung cancer when inhaled include, but are not limited to, asbestos, arsenic, aluminum, benzene, beryllium, cadmium, chromium and nickel compounds.

In some embodiments of the methods of the disclosure, the one or more risk factors for lung cancer comprises inhalation of radioactive materials. Radioactive materials of the disclosure can be inhaled as a gas or as particles. Exemplary radioactive materials include, but are not limited to radon (e.g. radon-222 and its decay products), plutonium-239 and radioactive cesium isotopes (e.g. cesium-134 and -137). For example, subjects who work or live near nuclear power plants are at an increased risk of exposure to radioactive substances through inhalation

In some embodiments of the methods of the disclosure, the one or more risk factors for lung cancer comprises a previous radiation therapy directed to the thorax, for example for the treatment of another cancer such as Hodgkin lymphoma or breast cancer. Radiation therapy targeting a cancer in the thorax causes genetic damage to proximal healthy lung cells, which leads to lung cancer. Radiation therapy to the chest increases the risk of the subsequent development of lung cancer. Radiation therapy can act synergistically with other risk factors, such as smoking, to increase the risk of developing lung cancer.

In some embodiments of the methods of the disclosure, the one or more risk factors for lung cancer comprises a familial history of lung cancer. Having a close family member (parent or sibling, e.g.) with lung cancer can increase the risk of developing lung cancer. A strong family history of lung cancer can indicate a genetic and heritable predisposition for the development of lung cancer. If, for example, the strong family history of lung cancer is not accompanied by a family history of smoking a shared exposure to environmental carcinogens, this can be evidence of a genetic, familial risk of lung cancer. Alternatively, or in addition, a strong family history of lung cancer can be due to shared environmental risk factors.

In some embodiments of the methods of the disclosure, the one or more risk factors for lung cancer comprises a previous occurrence of lung cancer. A previous occurrence of lung cancer, even if it is seemingly in complete remission, increases the risk of developing another lung cancer. In some embodiments, the second lung cancer is a recurrence of the first cancer. In some embodiments, the cancer is a new, unrelated cancer (a second cancer). In particular, survivors of non-small cell lung cancer are at increased risk of developing additional cancers, including additional lung cancers. Smoking acts synergistically with a previous lung cancer to increase the risk of developing a new lung cancer.

In some embodiments of the methods of the disclosure, the one or more risk factors for lung cancer comprises the detection of a lung nodule or mass. Methods of detection of lung abnormalities, and their use in the diagnosis of and screening for lung cancer will be well known to the ordinary skilled artisan. For example, lung nodules or masses can be detected by computed tomography (CT) scan or chest radiography. About 40% of pulmonary nodules turn out to be cancerous. Thus, the detection of a lung nodule or mass increases the risk of developing lung cancer. As referred to herein, a “suspicious nodule” is a nodule that, based on any of the imaging tests described herein or known in the art, has an appearance that is consistent with lung cancer.

In some embodiments of the methods of the disclosure, the one or more risk factors for lung cancer comprises one or more biomarkers associated with lung cancer. In some embodiments, the biomarker comprises an angiogenic factor, a lung cancer associated protein, an RNA, a DNA, a micro-RNA, an exosome, a circulating tumor cell or a change in metabolites. Subjects can be screened for lung cancer biomarkers using a variety of methods known in the art. These methods include, but are not limited to high throughput sequencing to identify the downregulation or upregulation of RNAs associated with lung cancer, immunohistochemistry methods, such as ELISAs, to identify protein expression associated with lung cancer, spectrometry of exhaled breathe to identify volatile organic compounds that reflect metabolic activity associated with lung cancer, and the purification and analysis of factors circulating in the blood such as exosomes or cancer cells.

Lung cancer risk factors of the disclosure can be determined by a variety of methods. For example, evaluating a patient's medical and family history can determine if there is a family history of lung cancer, or a history of smoking. Assessment of the patient's environment, e.g. at home or at work, can determine if there has been exposure to carcinogens or radioactive agents. Genetic testing for single nucleotide polymorphisms (SNPs) associated with lung cancer can determine if there is a genetic risk for lung cancer. All risk factors for lung cancers, and all methods for assessing those risk factors, are considered to be within the scope of the disclosure.

Testing subjects for IL-1 genotype allows for more intensive monitoring for early detection of lung cancer in IL-1 positive subjects. Testing subjects for IL-1 genotype allows early intervention with IL-1 inhibitors such as Canakinumab to prevent lung cancer in IL-1 positive subjects. Testing subjects for IL-1 genotype allows for early intervention with new genetically-directed treatment protocols that may combine drugs targeting somatic mutations such as EGFR, in combination with IL-1 blocking drugs. IL-1 positive subjects carry IL-1 variants that drive high production of IL-1, which activates EGFR expression (Lee, Syu et al. 2015), and so the need for EGFR targeted therapies may be particularly acute in subjects who are also IL-1 genotype positive.

### Lung Cancer Tests

The disclosure provides methods of reducing a risk of developing lung cancer in a subject comprising determining the subject's IL-1 genotype status; diagnosing the subject as at risk of developing lung cancer if the subject has a positive IL-1 genotype pattern and administering a non-genetic lung cancer test to the subject diagnosed as having an IL-1 positive genotype. The methods of the disclosure are thus able to reduce the risk of or prevent lung cancer by identifying IL-1 positive subjects who will derive a benefit from increased lung cancer screening using non-genetic lung cancer tests.

In some embodiments, the non-genetic lung cancer test is administered to a subject who would not otherwise receive it—e.g., a subject who is not thought to be at risk. In some embodiments, the non-genetic lung cancer test is administered more frequently to a subject who is IL-1 positive than to a subject who is IL-1 negative. In some embodiments, the testing is administered once a month, every 2 months, every 3 months, every 4 months, every 5 months, every 6 months, every 8 months, every 12 months, every 18 months, every 2 years, every 2.5 years or every 3 years.

In some embodiments, the non-genetic lung cancer test comprises an imaging test.

Imaging tests for lung cancer will be known to the person of ordinary skill in the art, and include chest X-rays, sputum cytology, magnetic resonance imaging (MRI) or fluorodeoxyglucose positron emission tomography computed tomography (PET/CT). In some embodiments, the chest X-ray comprises a low-dose spiral computed tomography (CT) scan, a low dose CT scan, or a low-dose helical CT scan.

In some embodiments, the non-genetic lung cancer test comprises a test for a lung cancer biomarker. Exemplary biomarkers for lung cancer include, but are not limited to an angiogenic factors, lung cancer associated proteins, an RNA, a DNA, a micro-RNA, an exosome, a circulating tumor cell or a change in metabolites.

In some embodiments, the lung cancer associated biomarker comprises a protein. The expression of lung cancer associated proteins can be assayed by any method known in the art, for example by immunohistochemistry methods such as ELISAs or antibody stains which use antibodies specific to the protein to detect its expression. Exemplary lung cancer associated proteins comprise advanced glycosylation end-product specific receptor (AGER), adipogenesis regulatory factor (C10orf116), adducin 2 (ADD2), periaxin (PRX), laminin subunit beta 3 (LAMB3), synemin (SYNM), spectrin alpha, erythrocytic 1 (SPTA1), ankyrin 1 (ANK1), hemoglobin subunit epsilon 1 (HBE1), hemoglobin subunit gamma 1 (HBG1), carbonic anhydrase 1 (CA1), tenascin XB (TNXB), multimerin 2 (MMRN2), hemoglobin subunit alpha 1 (HBA1), caveolin 1 (CAV1), hemoglobin subunit beta (HBB), collagen type VI alpha 6 chain (COL6A6), chromosome 1 open reading frame 198 (C1orf198), chloride intracellular channel 2 (CLIC2), caveolae associated protein 2 (SDPR), EH domain containing 2 (EHD2), apolipoprotein A2 (APOA2), NADH:ubiquinone oxidoreductase subunit B7 (NDUFB7), caveolae associated protein 3 (PRKCDBP), aminin subunit alpha 3 (LAMA3), EvC ciliary complex subunit 2 (LBN), four and a half LIM domains 5 (ACT), insulin like growth factor binding protein 3 (IGFBP3), prostaglandin D2 synthase (L-PGDS), serum amyloid A1 (SAA), retinoic acid receptor beta (HAP), hepatocyte growth factor (HGF), transthyretin (TTR), clusterin (CLU), tripartite motif containing 21 (SSA), apolipoprotein A4 (APOA4), ceruloplasmin (CP), haptoglobin (HP), keratin 2 (KRT2A), glutamate transporter 1b (GLT1B), casein kinase 1 alpha 1 (CK1), AKT serine/threonine kinase 1 (AKT), mannose binding lectin 2 (MBL2), tRNA-Leu (AAG) 1-2 (AAG1-2), fibrinogen alpha chain (FGA), gelsolin (GSN), ficolin 3 (FCN3), carnosine dipeptidase 1 (CNDP1), calcitonin related polypeptide alpha (CALCA), carbamoyl-phosphate synthase 1 (CPS1), chromogranin B (CHGB), involucrin (IVL), anterior gradient 2, protein disulphide isomerase family member (AGR2), nuclear autoantigenic sperm protein (NASP), phosphofructokinase, platelet (PFKP), thrombospondin 2 (THBS2), thioredoxin domain containing 17 (TXNDC17), proprotein convertase subtilisin/kexin type 1 (PCSK1), cellular retinoic acid binding protein 2 (CRABP2), acyl-CoA binding domain containing 3 (ACBD3), desmoglein 2 (DSG2), LPS responsive beige-like anchor protein (LRBA), serine/threonine kinase receptor associated protein (STRAP), VGF nerve growth factor (VGF), NOP2 nucleolar protein (NOP2), lipocalin 2 (LCN2), creatine kinase, mitochondrial 1B (CKMT1B), aldo-keto reductase family 1 member B10 (AKR1B10), proliferating cell nuclear antigen (PCNA), carboxypeptidase D (CPD), proteasome activator subunit 3 (PSME3), villin 1 (VIL1), serpin family B member 5 (SERPINB5), ribosomal protein L5 (RPL5), plakophilin 1 (PKP1), Ribosomal protein L10 (RPL10), aldo-keto reductase family 1 member C1 (AKR1C1), ribosomal protein S2 (RPS2), aldo-keto reductase family 1 member C3 (AKR1C3), visinin like 1 (VSNL1), adenosylhomocysteinase (AHCY), IMMP10, p21 (RAC1) activated kinase 2 (PAK2), isoleucyl-tRNA synthetase (IARS), proteasome 26S subunit, non-ATPase 2 (PSMD2), guanylate binding protein 5 (GBP5), minichromosome maintenance complex component 6 (MCM6), N-myc downstream regulated 1 (NDRG1), NOP58 ribonucleoprotein (NOP58), S100 calcium binding protein A2 (S100A2), Neuregulin 1 (NRG1), Neuregulin 2 (NRG2), ISG15 ubiquitin like modifier (UCRP), CER, plasminogen activator, urokinase (UPA), matrix metallopeptidase 14 (MT1-MMP), stratifin (SFN), transferrin (TF), albumin (ALB), S100 calcium binding protein A9 (S100A9), stathmin 1 (STMN), ENO, insulin like growth factor binding protein 7 (IGFBP7), or thrombospondin 1 (THBS1).

In some embodiments, the lung cancer biomarker comprises an angiogenic factor.

Angiogenic factors are factors that stimulate the proliferation and differentiation of cell types necessary for building blood vessels, including endothelial cells and smooth muscle cells. Angiogenic factors include growth factors such as fibroblast growth factor 1 (FGF-1) and vascular endothelial growth factor (VEGF). Any angiogenic factor associated with angiogenesis in lung cancer is envisaged as within the scope of the disclosure. In some embodiments, the angiogenic factors are proteins, RNAs or DNAs, and can be detected using the methods described herein.

In some embodiments, the lung cancer associated biomarker comprises an RNA, for example a messenger RNA of a gene associated with lung cancer or a microRNA. Exemplary genes associated with lung cancer, include, but are not limited to AGER, C10orf116, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, (SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, UPA, MT1-MMP, SFN, TF, ALB, S100A9, STMN, ENO, IGFBP7, or THBS1. Methods of measuring RNAs are known in the art, and include RT-PCR, microarrays and high throughput sequencing.

In some embodiments, the lung cancer biomarker comprises a change in metabolites. Changes in metabolites associated with lung cancer can be detected by a variety of methods known in the art. For example, changes in metabolites can be detected by metabolomic profiling of lung biopsy samples, or bio fluids such as blood, plasma or urine. Alternatively, or in addition, metabolites from exhaled breathe samples can be assayed for changes indicative of lung cancer. Metabolites in sample can be measured by methods known in the art, such as liquid chromatography, mass spectrometry, or a combination thereof.

### Colorectal Cancer

The disclosure provides methods of reducing a risk of developing colorectal cancer in a subject comprising: determining whether the subject is IL-1 genotype positive or IL-1 genotype negative using the methods described herein, and administering a non-genetic colorectal cancer test to the subject diagnosed as having an IL-1 positive genotype.

The disclosure further provides methods of treating colorectal cancer in a subject comprising: determining whether the subject is IL-1 genotype positive or IL-1 genotype negative using the methods described herein, and administering an IL-1 inhibitor to the subject diagnosed as having an IL-1 positive genotype. In some embodiments, identifying a subject who has colorectal cancer comprises: (i) identifying a subject who has one or more risk factors or biomarkers of colorectal cancer; and (ii) testing the subject for colorectal cancer using the methods described herein.

In some embodiments, the subject does not have a risk factor for colorectal cancer. The methods of the current disclosure, by identifying subjects who are IL-1 positive, is able to identify subjects outside of conventional “at-risk” populations for colorectal cancer target these subjects for additional screening and other preventative measures. For example, subjects who are IL-1 positive but have not have colon polyps when screened can be targeted for additional monitoring using the methods described herein.

Colorectal cancer is a cancer that starts in the colon or rectum. Colorectal cancers can also be termed colon or rectal, depending on where the cancer originated. In many cases, colorectal cancer is associated with the development of polyps, which are abnormal growths that start in the inner lining of the colon or rectum. Polyps can have a stalk, or be flat, and the latter are difficult to identify in conventional screening methods such as colonoscopies. As referred to herein, “suspicious polyps” are polyps whose appearance is consistent with colorectal cancer, or polyps leading to colorectal cancer.

In some embodiments, the subject has one or more risk factors for colorectal cancer. Risk factors for colorectal cancer include, but are not limited to, being overweight or obese, lack of physical activity, diet, smoking, heavy alcohol use, age over fifty, a history of adenomatous polyps, a family history of adenomatous polyps, a previous diagnosis of colorectal cancer, a family history of colorectal cancer, a history of inflammatory bowel disease, type II diabetes, radiation therapy to treat prostate cancer or a genetic predisposition to colorectal cancer.

In some embodiments, the one or more risk factors for colorectal cancer comprises a genetic predisposition to colorectal cancer. Genetic predispositions for colorectal cancer include Lynch syndrome, familial adenomatous polyposis (FAP), and mutations in genes such as in serine/threonine kinase 11 (LBK1), mutY DNA glycosylase (MUTYH) or SMAD family member 4 (SMAD4). Lynch syndrome, also known as hereditary non-polyposis colorectal cancer, is an inherited cancer syndrome that can be caused by mutations in mutL homolog 1 (MLH1) or a mutS homolog 2 (MSH2). FAP is an inherited disorder characterized by cancer of the colon and rectum. People with FAP may begin to develop benign polyps in the colon as early as their teens, which can then turn cancerous. FAP can be caused by mutations in the adenomatous polyposis coli (APC) gene.

In some embodiments, the one or more risk factors for colorectal cancer comprises diet. Diets high in red and/ or processed meat are associated with the development of colorectal cancer.

The methods of the instant disclosure include administering a non-genetic colorectal cancer test to subjects diagnosed as having an IL-1 positive genotype. Non-genetic tests for colorectal cancer will be known to the person of ordinary skill in the art. Exemplary non-genetic tests for colorectal cancer include a high-sensitivity fecal occult blood test (FOBT), a fecal immunochemical test (FIT), a sigmoidoscopy, a colonoscopy, computed tomographic (CT) colonography, a double contrast barium enema or a blood test. Both FOBT and FIT are tests that can be used evaluate stool samples for blood that can be caused by the presence of polyps or cancers in the colon, for example using immunoassays that test for the presence of hemoglobin or other blood markers. FIT can also test for the presence of DNA markers associated with colorectal cancer in stool. A sigmoidoscopy is an examination procedure that is used to examine the lower 20 inches of a subject's colon and rectum, thereby screening colorectal cancer and polyps. A flexible sigmoidoscope, usually with a video camera, is inserted into the rectum. computed tomographic (CT) colonography uses special X-ray equipment to examine to colon for cancer and polyps.

In some embodiments, the non-genetic colorectal cancer test comprises testing for one or more biomarkers associated with colorectal cancer. Colorectal biomarkers include DNA markers associated with colorectal cancer, for example DNA markers in stool samples. Without wishing to be bound by theory, it is thought that colorectal cancers shed cells, and that these cells and the colorectal cancer DNA markers of these cells can be detected in stool samples. Exemplary DNA markers comprise an alteration in APC, catenin beta 1 (CTNNB1), KRAS proto-oncogene, GTPase (KRAS), B-Raf proto-oncogene, serine/threonine kinase (BRAF), SMAD4, transforming growth factor beta receptor 2 (TGFBR2), tumor protein p53 (TP53), phosphatidylinositol-4,5-bisphosphate 3-kinase catalytic subunit alpha (PIK3CA), AT-rich interaction domain 1A (ARID1A), SRY-box 9 (SOX9), APC membrane recruitment protein 1 (FAM123B), erb-b2 receptor tyrosine kinase 2 (ERBB2), vimentin (VIM), NDRG family member 4 (NDRG4), septin 9 (SEPT9), bone morphogenetic protein 3 (BMP3) or tissue factor pathway inhibitor 2 (TFPI2). In some embodiments, the alteration in the DNA marker comprises a mutation, such as an insertion, deletion, rearrangement or substitution. In some embodiments, the alteration comprises a change in DNA methylation. In some embodiments, the biomarker comprises methylated SEPT9 DNA.

In some embodiments, the non-genetic colorectal cancer test is administered to a subject who would not otherwise receive it—e.g., a subject who is not thought to be at risk. In some embodiments, the non-genetic colorectal cancer test is administered more frequently to a subject who is IL-1 positive than to a subject who is IL-1 negative. In some embodiments, the testing is administered once a month, every 2 months, every 3 months, every 4 months, every 5 months, every 6 months, every 8 months, every 12 months, every 18 months, every 2 years, every 2.5 years or every 3 years.

The disclosure further provides methods of treating colorectal cancer in a subject comprising: determining whether the subject is IL-1 genotype positive or IL-1 genotype negative using the methods described herein, and administering an IL-1 inhibitor to the subject diagnosed as having an IL-1 positive genotype.

### Breast Cancer

IL-1β from both the tumor cells and the tumor microenvironment influences the growth of primary breast tumors, dissemination of breast cancer cells into the bone metastatic niche, and proliferation into overt metastases. IL-1β is a pro-inflammatory cytokine whose expression in primary tumors has been identified as a biomarker for predicting breast cancer patients at increased risk for developing bone metastasis. Breast cancer cells with increased ability to metastasize in bone (MDA-IV) have higher IL-1β expression, compared to a corresponding parental line, indicating that IL-1β expression enhances metastatic potential (Nutter et al. 2014).

Accordingly, the disclosure provides methods of reducing a risk of metastatic breast cancer in a subject comprising: identifying a subject who has breast cancer, determining whether the subject is IL-1 genotype positive or negative using the methods described herein, diagnosing the subject as at risk for metastatic breast cancer if the subject has a positive IL-1 genotype pattern, and administering an IL-1 inhibitor to the subject to the subject diagnosed with a positive IL-1 genotype pattern.

Breast cancer is a type of cancer that forms in the cells of the breasts, and is the second most common cancer diagnosed in women the U.S. Breast cancer occurs in both men and women, but is more frequently seen in women. Types of breast cancer include carcinoma, sarcoma, Phyllodes tumor, Paget disease, angiosarcoma and inflammatory breast cancer. All types of breast cancer are within the scope of the instant disclosure.

In some embodiments, the breast cancer is a stage 0, stage I, stage II or stage III breast cancer.

Subjects who are IL-1 positive and have breast cancer can be treated with any of the IL-1 inhibitors and methods described herein. In some embodiments, administering the IL-1 inhibitor reduces a sign or a symptom of the breast cancer. In some embodiments, administering the IL-1 inhibitor reduces metastasis of the breast cancer. Without wishing to be bound by theory, it is thought that inflammation can contribute to angiogenesis through the upregulation of pro-angiogenic factor such as VEGF, such as contribute to breast cancer metastasis. Reducing inflammation in subjects who are IL-1 positive by administering an IL-1 inhibitor described herein can reduce angiogenesis, and thereby metastasis of breast cancers. Alternatively, or in addition, administering IL-1 inhibitors to IL-1 positive subjects can reduce lymph node metastasis, invasion, and tumor differentiation, thereby reducing the risk of metastasis.

### Treatment of Cancer

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

In some embodiments of the methods of the disclosure, the method further comprises an additional therapy for cancer. All cancer therapies are combinable with the IL-1 genotyping methods and IL-1 inhibitors described herein.

For example, a subject diagnosed with lung, colorectal or breast cancer and who has an IL-1 positive genotype pattern according to Tables 1-3 will be administered both an IL-1β inhibitor such as canakinumab and one or more of chemotherapy, radiation treatment, surgical removal of the cancer, an immunotherapy, an antibody therapy, an immune checkpoint inhibitor, a therapeutic vaccine, or a combination thereof. For example, the IL-1β inhibitor is Canakinumab.

Surgery is a preferred treatments for patients with early stage (e.g., non-metastatic) lung cancers. Surgery for lung cancer can involve removal of part or all of the lung. Surgery for breast cancer can involve removal of part or all of the breast. Surgery for colorectal cancer can remove part of the colon. Wedge resections remove only the tumor and a small portion of healthy tissue, while, for lung cancer, lobectomies remove one of the lung's lobes and pneumonectomies remove the entire lung. The appropriate surgical intervention will depend on the type, position, and stage of the cancer. Surgery is frequently combined with other cancer therapies of the disclosure such as radiation, chemotherapy, antibody therapy and immune checkpoint inhibitors.

Radiation therapy can be used to treat cancers of the disclosure. Radiation therapy can be delivered from an external source, such as external beam radiation therapy (EBRT). Alternatively, radiation can be delivered via an implant placed close to or inside the tumors in the body (internal radiation). An example of the latter sort of therapy is high dose rate (HDR) brachytherapy. Radiation is frequently combined with other lung cancer therapies of the disclosure such as chemotherapy, antibody therapy and immune checkpoint inhibitors. The appropriate radiation therapy will depend on the type, position, and stage of the cancer.

In some embodiments of the methods of the disclosure, the methods further comprise a chemotherapy. For example, a subject who is diagnosed with lung, colorectal or breast cancer and has a positive IL-1 genotype according to Tables 1-3 can be administered an IL-1β inhibitor such as canakinumab and a chemotherapeutic agent from Table 7. Optionally, the chemotherapeutic agent and the IL-1β inhibitor are combined with one or more additional lung cancer therapies.

Chemotherapies interfere with the ability of cancer cells to grow and divide. Chemotherapies of the disclosure include, but are not limited to, DNA damaging agents such as alkylating agents, antimetabolites, alkaloids, mitotic inhibitors, topoisomerase inhibitors, antitumor antibiotics, tyrosine kinase inhibitors, mTOR inhibitors, a B-Raf inhibitors, EGFR inhibitors, PARP inhibitors, phosphoinositide 3-kinase (PI3K) inhibitors, CDK inhibitors or a combination thereof.

Antimetabolites include, for example, folic acid, pyrimidine and purine analogues, and can interfere with the enzymatic reactions in cancer cells. Exemplary antimetabolites include but are not limited to methotrexate and gemcitabine.

Alkaloids attack cancer cells during various phases of the cell cycle. Alkaloid chemotherapies include, but are not limited to, vinca alkaloids such as vincristine, vinblastine and vinorelbine, taxanes such as paclitaxel and docetaxel, podophyllotoxins such as etoposide and teniposide and camptothecan analogs such as irinotecan and topotecan.

Topoisomerase inhibitors interfere with the action of topoisomerase enzymes, which are critical for successful DNA replication. Exemplary topoisomerases include, but are not limited to, irinotecan, topotecan, etoposide, etoposide phosphate and teniposide.

Antitumor antibiotics slow or stop the growth of cancer cells. Exemplary antitumor antibiotics include doxorubicin, mitoxantrone or bleomycin.

Taxanes bind to microtubules and interfere with cancer cell division. Exemplary taxanes include taxol, docetaxel or paclitaxel.

Platinum-based agents are important drugs or drug candidates for cancer chemotherapy. Exemplary platinum agents comprise cisplatin, oxaliplatin or carboplatin.

B-Raf kinase dysregulation has been implicated in a number of cancers, including colorectal cancer. Exemplary B-Raf inhibitors include dabrafenib.

EGFR signaling plays an important role in cell proliferation, survival, gene expression and apoptosis. EGFR signaling has been implicated in the progression of a number of cancers. For example, mutations in members of the EGFR pathway are frequently found in lung cancers, and mutations in the EGFR family member HER2 are associated with aggressive breast cancer. EGFR inhibitors include erlotinib, gefetinib and osimertinib.

Some cancers are more dependent on poly-ADP ribose polymerase (PARP) than regular cells. For example, BRCA1, BRCA2 or triple negative breast cancers can be susceptible to PARP inhibitors. Exemplary PARP inhibitors include veliparib, olaparib and talazoparib.

The phosphoinositide 3-kinase (PI3K) pathway can frequently be upregulated in cancer cells such as breast, lung and colorectal cancer, and can be targeted in cancer therapies. Exemplary PI3K inhibitors include pubarlisib.

Tyrosine kinase inhibitors inhibit the activity of tyrosine kinases, which can be important mediators of the cell proliferation, differentiation, migration and metabolism of cancer cells. Exemplary tyrosine kinase inhibitors include afatinib, apatinib, alectinib, brigantinib, ceritinib, CDX-301, crizotinib, trametinib, selumetinib, lapatinib, neratinib and sunitinib.

mTOR, or target of rapamycin, plays a key role in cell growth and proliferation, and the inhibition of mTOR can treat certain cancers, including lung, breast and colorectal cancers. Exemplary mTOR inhibitors include everolimus.

Mitotic inhibitors are drugs that inhibit mitosis, or cell division, frequently by disrupting microtubule structure. Exemplary mitotic inhibitors include, but are not limited to, ixabepilone, paclitaxel and eribulin.

Cyclin Dependent Kinase inhibitors (CDK) inhibit cyclin dependent kinases, and can be used to inhibit cellular proliferation. CDK inhibitors can be used to many cancers, including lung, colorectal and breast cancer. For example, CDK4/6 inhibitors such as abemaciclib, palbociclib and ribocliclib can be used to treat metastic breast cancer.

Chemotherapies of the disclosure include, but are not limited to, paclitaxel, paclitaxel albumin-stabilized nanoparticle formulation, afatinib dimaleate, apatinib, alectinib, everolimus, pemetrexed disodium, brigantinib, cisplatin, carboplatin, ceritinib, crizotinib, CDX-301, dabrafenib, docetaxel, erlotinib hydrochloride, irinotecan, indoximod, gefetinib, gemcitabine hydrochloride, mechlorethamine hydrochloride, trametinib, methotrexate, vinorelbine tartrate, osimertinib, taxol, doxorubicin, doxorubicin hydrochloride, etoposide, etoposide phosphate, topotecan hydrochloride, vinblastine, veliparib, olaparib, buparlisib, selumetinib, sunitinib or a combination thereof. A list of exemplary, but non-limiting chemotherapeutic agents is disclosed in Table 7:

TABLE 7
Chemotherapies

| Name | Brand Name ® |
|---|---|
| paclitaxel | Onxol, Taxol |
| paclitaxel albumin-stabilized | Abraxane |
| nanoparticle formulation | |
| afatinib dimaleate | Gilotrif |
| apatinib | Rivoceranib |
| alectinib | Alecensa |
| everolimus | Afinitor, Zortress, Afinitor |
| | Disperz |
| pemetrexed disodium | Alimta |
| brigantinib | Alunbrig |
| cisplatin | Platinol |
| carboplatin | Paraplatin |
| ceritinib | Zykadia |
| crizotinib | Xalkori |
| CDX-301 | |
| dabrafenib | Tafinlar |
| docetaxel | Docefrez, Taxotere |
| erlotinib hydrochloride | Tarceva |
| irinotecan | Camptosar, Onivyde |
| indoximod | |
| gefetinib | Iressa |
| gemcitabine hydrochloride | Gemza |
| mechlorethamine hydrochloride | Mustargen, Valchlor |
| trametinib | Mekinist |
| methotrexate | Trexall, Rasuva, Otrexup, |
| | Xatmep |
| vinorelbine tartrate | Navelbine |
| osimertinib | Tagrisso |
| doxorubicin, doxorubicin hydrochloride | Adriamycin, Doxil, Lipidox, |
| | Myocet, Rubex |
| etoposide, etoposide phosphate | Etopophos, Toposar |
| topotecan hydrochloride | Hycamtin |
| vinblastine | Velban |
| veliparib | |
| olaparib | Lynparza |
| buparlisib (BKM120) | |
| Selumetinib (AZD6244) | |
| sunitinib | Sutent |

In some embodiments of the methods of the disclosure, the methods further comprise an antibody therapy. For example, a subject who is diagnosed with lung cancer and has a positive IL-1 genotype according to Tables 1-3 can be administered an IL-1β inhibitor such as canakinumab and an antibody therapy from Table 8. In some embodiments, the IL-1β and the antibody therapy can be combined with more or more additional lung cancer therapies such as the chemotherapies disclosed in Table 7, as well as surgery and/ or radiation.

In some embodiments of the methods of the disclosure, including those embodiments wherein the method further comprises an antibody therapy, the antibody therapy comprises APX005M, avelumab, bavituximab, bevacizumab, cetuximab, cetuximab, conatumumab, durvalumab, denosumab, dalotuzumab, ficlatuzumab, figitumumab, fresolimumab, Hu3S193, ipilimumab, MN-14, mapatumuzab, matuzumab, MEDI4736, necitumumab, nivolumab, nimotuzumab, nofetumomab, olaratumab, onartuzumab, pembrolizumab, panitumumab, pertuzumab, racotumomab, ramucirumab, rovalpituzumab, tucotuzumab, tremelimumab, trastuzumab, zalutumumab or a combination thereof. Exemplary, but not limiting antibodies used in antibody therapies of the disclosure are disclosed in Table 8:

TABLE 8
Antibodies

| Name | Brand Name ® |
|---|---|
| APX005M | |
| avelumab | Bavencio |
| Bavituximab (PGN401) | |
| bevacizumab | Avastin |
| cetuximab | Erbitux |
| conatumumab | |
| durvalumab | Imfinzi |
| denosumab | Xgeva, Prolia |
| Dalotuzumab (MK-0646) | |
| Ficlatuzumab (AV-299) | |
| figitumumab | |
| Fresolimumab (GC1008) | |
| Hu3S193 | |
| ipilimumab | Yervoy |
| MN-14 | |
| mapatumuzab | |
| Matuzumab (EMD72000) | |
| MEDI4736 | |
| necitumumab | Portrazza |
| nivolumab | Opdivo |
| nimotuzumab | Theracim, Theraloc, Biomab |
| | EGFR |
| nofetumomab | Verluma |
| olaratumab | Lartruvo |
| onartuzumab | |
| pembrolizumab | Keytruda |
| panitumumab | Vectibix |
| pertuzumab | Perjeta |
| racotumomab | |
| ramucirumab | Cyramza |
| rovalpituzumab | Rova-T |
| tucotuzumab | |
| tremelimumab | |
| trastuzumab | Herceptin |
| zalutumumab | |

In some embodiments of the methods of the disclosure, the methods further comprise an immune checkpoint inhibitor. For example, a subject who is diagnosed with lung, colorectal or breast cancer and has a positive IL-1 genotype according to Tables 1-3 may be administered an IL-1 inhibitor such as canakinumab and an immune checkpoint inhibitor from Table 9. In some embodiments, the IL-1 inhibitor and the antibody therapy can be combined with more or more additional cancer therapies such as the chemotherapeutic agents disclosed in Table 7, and the antibody therapies disclosed in Table 8, as well as surgery and/ or radiation.

Immune checkpoints are regulators of the immune system. Immune checkpoints play a critical role in maintaining self-tolerance, preventing autoimmunity and protecting tissues from damage from the immune system. Immune checkpoints can function by downregulating the immune system. Negative immune checkpoints are frequently co-opted by tumors to inhibit the ability of the immune system to mount an effective immune response to the tumor. Blocking negative regulators of immune checkpoints thus allows for the activation of anti-cancer immunity. Immune checkpoints comprise the programmed cell death 1 (PD-1) checkpoint, the CD274 molecule (PD-L1) checkpoint and the cytotoxic T-lymphocyte associated protein 4 (CTLA-4) checkpoint. In some embodiments of the methods of the disclosure, the immune checkpoint inhibitor comprises a programmed cell death 1 (PD-1) inhibitor, a CD274 molecule (PD-L1) inhibitor or a cytotoxic T-lymphocyte associated protein 4 (CTLA-4) checkpoint inhibitor. Exemplary but non-limiting PD-1 inhibitors comprise nivolumab and pembrolizumab. Exemplary but non-limiting PD-L1 inhibitors comprise atezolizumab, avelumab and durvalumab. Exemplary but non-limiting CLTA-4 inhibitors comprise ipilimumab. In some embodiments, the immune checkpoint inhibitor comprises atezolizumab, avelumab, durvalumab, ipilimumab, tremelimumab, indiximod, nivolumab, pembrolizumab or a combination thereof. Exemplary but non-limiting immune checkpoint inhibitors are disclosed in Table 9:

| Name | Brand Name ® |
|---|---|
| atezolizumab | Tecentriq |
| avelumab | Bavencio |
| durvalumab | Imfinzi |
| ipilimumab | Yervoy |
| tremelimumab | |
| indiximod | |
| nivolumab | Opdivo |
| pembrolizumab | Keytruda |

Any drug of Tables 4-6 may be administered with any other drug or drugs known in the art that is capable of treating or reducing a sign or a symptom of one or more of the diseases or disorders relevant to the present invention, such as lung, colorectal or breast cancer.

Canakinumab may be administered with any other drug or drugs known in the art that is capable of treating or reducing a sign or a symptom of one or more diseases or disorders relevant to the present invention, such as lung cancer.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “monotherapy” refers to the administration of a single active or therapeutic compound to a subject in need thereof. Preferably, monotherapy will involve administration of a therapeutically effective amount of an active compound. For example, cancer monotherapy with one of the compound of the present invention, or a pharmaceutically acceptable salt, polymorph, solvate, analog or derivative thereof, to a subject in need of treatment of cancer. Monotherapy may be contrasted with combination therapy, in which a combination of multiple active compounds is administered, preferably with each component of the combination present in a therapeutically effective amount. In one aspect, monotherapy with a compound of the present invention, or a pharmaceutically acceptable salt, polymorph or solvate thereof, is more effective than combination therapy in inducing a desired biological effect.

In an embodiment, any drug of Tables 4-6 may be administered in combination with any of the drugs listed in Tables 7-9, wherein the drug listed in Tables 7-9 is used as a monotherapy for a cancer of the disclosure. For example, canakinumab may be administered with one of the drugs listed in Tables 7-9.

Alternatively, any drug of Tables 4-6 may be administered together with a combination therapy for a cancer of the disclosure. The combination therapy may comprise one or more of the drugs listed in Tables 7-9. For example, canakinumab may be administered together with a combination therapy for lung, colorectal or breast cancer. The combination therapy may comprise one or more of the drugs listed in Tables 7-9.

As used herein, the terms “prevent,” “preventing,” “prevention,” “prophylactic treatment” and the like refer to reducing the probability of developing a disorder or condition in a subject, who does not have, but is at risk of or susceptible to developing a disorder or condition.

As used herein, the terms “treat,” treating,” “treatment,” and the like refer to reducing or ameliorating a disorder and/ or a symptom associated therewith. It will be appreciated that, although not precluded, treating a disorder or condition does not require that the disorder, condition or symptoms associated therewith be completely eliminated. Treating may include a health care professional or diagnostic scientist making a recommendation to a subject for a desired course of action or treatment regimen, e.g., a prescription. It should be noted that “Treating” or “treat” describes the management and care of a patient for the purpose of combating a disease, condition, or disorder and includes the administration of a compound of the present invention, or a pharmaceutically acceptable salt, polymorph or solvate thereof, to alleviate one or more symptoms or complications of a disease, condition or disorder, or to eliminate the disease, condition or disorder. The term “treat” can also include treatment of a cell in vitro or an animal model.

A compound of the present invention, or a pharmaceutically acceptable salt, polymorph or solvate thereof, can also be used to prevent a disease, condition or disorder, or used to identify suitable candidates for such purposes. As used herein, “preventing” or “prevent” describes reducing or eliminating the onset of the symptoms or complications of the disease, condition or disorder. “Prevent” or “preventing” also describes reducing the probability, or risk, of developing a sign or a symptom of a disease of the disclosure.

As used herein, the term “alleviate” is meant to describe a process by which the severity of a sign or symptom of a disorder is decreased. Importantly, a sign or symptom can be alleviated without being eliminated. In a preferred embodiment, the administration of pharmaceutical compositions of the invention leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

In another aspect of the invention, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/ or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the subject experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Treating cancer can result in a reduction in size of a tumor. A reduction in size of a tumor may also be referred to as “tumor regression”. Preferably, after treatment, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer can result in a reduction in tumor volume. Preferably, after treatment, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer results in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer can result in a decrease in number of metastatic lesions in other tissues or organs distant from the primary tumor site. Preferably, after treatment, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in increase in average survival time of a population of treated subjects in comparison to a population receiving monotherapy with a drug that is not a compound of the present invention, or a pharmaceutically acceptable salt, polymorph, solvate, analog or derivative thereof. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a compound of the present invention, or a pharmaceutically acceptable salt, polymorph, solvate, analog or derivative thereof. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active compound. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In an aspect, cell death occurs by apoptosis.

A cancer that is to be treated can be staged according to the American Joint Committee on Cancer (AJCC) TNM classification system, where the tumor (T) has been assigned a stage of TX, Tl, Tlmic, Tla, Tib, Tic, T2, T3, T4, T4a, T4b, T4c, or T4d; and where the regional lymph nodes (N) have been assigned a stage of NX, NO, N1, N2, N2a, N2b, N3, N3a, N3b, or N3c; and where distant metastasis (M) can be assigned a stage of MX, M0, or Ml. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pNO, PNO (I−), PNO (I+), PNO (mol−), PNO (mol+), PN1, PN1(mi), PN1a, PN1b, PN1c, pN2, pN2a, pN2b, pN3, pN3a, pN3b, or pN3c.

Lung cancers of the disclosure can be divided into the 4 stages as described by the AJCC. In stage TX, the primary tumor cannot be assessed, but the existence of the tumor is proven by the presence of malignant cells in sputum or bronchial washings although not visualized by imaging or bronchoscopy. In stage T0, there is no evidence of primary tumor. Stage Tis is a carcinoma in situ. In stage T1, the tumor is 3 cm or less in its greatest dimension, and is surrounded by lung or visceral pleura, and there is no evidence of invasion more proximal than the lobar bronchus. Stage T1 is divided into: stage T1a, in which the tumor is 2 cm or less in its greatest dimension; and stage T1b, in which the tumor is more than 2 cm but less than 3 cm or less in its greatest dimension. In stage T2, the tumor is more than 3 cm but less 7 cm and comprises any of the following features: the tumor involves main bronchus, the tumor is 2 cm or more distal to the carina; the tumor invades visceral pleura (PL1 or PL2); or the tumor is associated with atelectasis or obstructive pneumonitis that extends to the hilar region but does not involve the entire lung. A tumor is classified as T2a if the tumor is more than 3 cm but 5 cm or less in its greatest dimension. A tumor is classified as T2b if the tumor is more than 5 cm but 7 cm or less in its greatest dimension. In stage T3, the tumor is more than 7 cm, or directly invades any of the following: parietal pleural (PL3), chest wall (including superior sulcus tumors), diaphragm, phrenic nerve, mediastinal pleura, parietal pericardium; or the tumor is in the main bronchus and less than 2 cm distal to the carina, but does not involve carina; or there is associated atelectasis or obstructive pneumonitis of the entire lung or separate tumor nodule(s) in the same lobe. In stage T4, the tumor is of any size, and invades any of the following: mediastinum, heart, great vessels, trachea, recurrent laryngeal nerve, esophagus, vertebral body, carina, separate tumor nodule(s) in a different ipsilateral lobe.

All stages of lung cancer are envisaged as being treated by the compositions and methods of the disclosure.

Breast cancers of the disclosure can be divided into 5 stages. At stage T0, or precancerous, there is no evidence of the cancer in the breast. At stage T1, the tumor is 20 millimeters (mm) or smaller in size at its widest point. At stage T2, the tumor is between 20 and 50 mm. At stage T3 the tumor is larger than 50 mm. At stage T4, or metastatic breast cancer, the tumor can be classified as T4a, having gown into the chest wall; T4b, when the tumor has grown into the skin; T4c, when the cancer has grown into the chest wall and the skin; and T4d, inflammatory breast cancer.

All stages of breast cancer are envisaged as being treated by the compositions and methods of the disclosure.

Colorectal cancers of the disclosure can be divided into 5 stages. At stage T0, carcinoma in situ, the cancer has not moved from the point of origin and is still restricted to the innermost lining of the colon. At stage T1, also called Dukes A colon cancer, the cancer has begun to spread but is still in the inner lining. At stage T2, also called Dukes B colon cancer, the cancer may have gown through the wall of the colon into nearby tissue but has not yet spread to the lymph nodes. At stage T3, also called Dukes C colon cancer, the cancer has spread to nearby lymph nodes. At stage T4, also called Dukes D colon cancer, the cancer has spread through the lymph system to distant parts of the body (metastasis).

A drug is prepared depending in its route of drug administration. Examples of drug administration routes that are useful in the present invention are described on the U.S. Food and Drug Administration's website at the World Wide Web (www.fda.gov/Drugs/DevelopmentApprovalProcess/FormsSubmissionRequirements/ElectronicS ubmissions/DataStandardsManualmonographs/ucm071667.htm).

Preparations for oral administration generally contain inert excipients in addition to the active pharmaceutical ingredient. Oral preparations may be enclosed in gelatin capsules or compressed into tablets. Common excipients used in such preparations include pharmaceutically compatible fillers/diluents such as microcrystalline cellulose, hydroxypropyl methylcellulose, starch, lactose, sucrose, glucose, mannitol, sorbitol, dibasic calcium phosphate, or calcium carbonate; binding agents such as alginic acid, carboxymethylcellulose, microcrystalline cellulose, gelatin, gum tragacanth, or polyvinylpyrrolidone; disintegrating agents such as alginic acid, cellulose, starch, or polyvinylpyrrolidone; lubricants such as calcium stearate, magnesium stearate, talc, silica, or sodium stearyl fumarate; glidants such as colloidal silicon dioxide; sweetening agents such as sucrose or saccharin; flavoring agents such as peppermint, methyl salicylate, or citrus flavoring; coloring agents; and preservatives such as antioxidants (e.g., vitamin A, vitamin C, vitamin E, or retinyl palmitate), citric acid, or sodium citrate. Oral preparations may also be administered as aqueous suspensions, elixirs, or syrups. For these, the active ingredient may be combined with various sweetening or flavoring agents, coloring agents, and, if so desired, emulsifying and/ or suspending agents, as well as diluents such as water, ethanol, glycerin, and combinations thereof.

For parenteral administration (including subcutaneous, intradermal, intravenous, intramuscular, and intraperitoneal), the preparation may be an aqueous or an oil-based solution. Aqueous solutions may include a sterile diluent such as water, saline solution, a pharmaceutically acceptable polyol such as glycerol, propylene glycol, or other synthetic solvents; an antibacterial and/ or antifungal agent such as benzyl alcohol, methyl paraben, chlorobutanol, phenol, thimerosal, and the like; an antioxidant such as ascorbic acid or sodium bisulfite; a chelating agent such as ethylenediaminetetraacetic acid (EDTA); a buffer such as acetate, citrate, or phosphate; and/ or an agent for the adjustment of tonicity such as sodium chloride, dextrose, or a polyalcohol such as mannitol or sorbitol. The pH of the aqueous solution may be adjusted with acids or bases such as hydrochloric acid or sodium hydroxide. Oil-based solutions or suspensions may further comprise sesame, peanut, olive oil, or mineral oil.

For topical (e.g., transdermal or transmucosal) administration, penetrants appropriate to the barrier to be permeated are generally included in the preparation. Transmucosal administration may be accomplished through the use of nasal sprays, aerosol sprays, tablets, or suppositories, and transdermal administration may be via ointments, salves, gels, patches, or creams as generally known in the art. Topical ocular formulations, e.g., eye drops and eye ointments, are considered.

The amount of agent that is administered to the subject can and will vary depending upon the type of agent, the subject, and the particular mode of administration. Those skilled in the art will appreciate that dosages may also be determined with guidance from Goodman & Gilman's The Pharmacological Basis of Therapeutics, Twelfth Edition (2011), Appendix II, pp. 1891-1991, and the Physicians' Desk Reference 70^{th} Edition, 2016.

### Pharmacogenomics

Pharmacogenomics is the methodology which associates genetic variability with physiological and clinical responses to a drug. Pharmacogenetics is a subset of pharmacogenomics and is defined as “the study of variations in DNA sequence as related to drug response” (ICH E15; see the World Wide Web www.fda.gov/downloads/RegulatoryInformation/Guidances/ucm129296.pdf). Pharmacogenetics often focuses on genetic polymorphisms in genes related to drug metabolism, drug mechanism of action, underlying disease type, and drug associated side effects. Pharmacogenetics is the cornerstone of Personalized Medicine which allows the development and the targeted use of drug therapies to obtain effective and safe treatment, as well as to adjust existing treatment regimens to further optimize the efficacy and safety profile for the individual patient.

Pharmacogenetics has become a core component of many drug development programs, being used to explain variability in drug response among subjects in clinical trials, to address unexpected emerging clinical issues, such as adverse events, to determine eligibility for a clinical trial (pre-screening) to optimize trial yield, to develop drug companion diagnostic tests to identify patients who are more likely or less likely to benefit from treatment or who may be at risk of adverse events, to provide information in drug labels to guide physician treatment decisions, to better understand the mechanism of action or metabolism of new and existing drugs, and to provide better understanding of disease mechanisms as associated with treatment response.

Generally, pharmacogenetics analyses are often performed using the candidate genes research technique, which is a hypothesis-driven approach, based on the detection of polymorphisms in candidate genes pre-selected using knowledge of the disease, the drug's mode of action, toxicology, or metabolism of the drug.

### Clinical Indicators

In the present invention, using the candidate genes research technique, a subject has his/her composite IL-1 genotype or IL-1 genotype pattern determined (as disclosed herein). Additionally, s/he may have one or more risk factors as described herein.

Based on the combination of risk factors, diagnosis and the subject's IL-1 genotype pattern a more aggressive and optimal therapeutic intervention will be determined.

A subject may be administered a higher dose or a lower dose (e.g., the dose of a single treatment and/ or a daily dose comprising one or more single treatments) of a particular drug depending on his/her composite IL-1 genotype or IL-1 genotype pattern; alternately, the subject may be not given the particular drug depending on his/her composite IL-1 genotype or IL-1 genotype pattern and instead may be administered another drug. For example, the other drug may operate by a different mode of action.

Alternately, the present invention may be used to optimize the size of a clinical trial.

For this, a study population is stratified by IL-1 pattern during or before randomization. This way, each group in a study will have sufficient numbers of members from each Pattern. This allows for smaller-sized groups which can nonetheless be informative and provide statistical significance. Non-Caucasian ethnic/racial groups have different frequencies for each pattern; thus, study populations comprising Non-Caucasians may need to have their total population size adjusted accordingly.

Such stratification of clinical trial subjects may occur any time before, during, or after the clinical trial. In the latter case, for example, if a clinical trial does not provide statistical significance using a general, non-stratified population, true statistical significant may be later be discovered when the subject data is reconsidered and stratified by IL-1 pattern. That is, if the data of the clinical did not show statistical evidence of a treatment response, the data could later be revaluated with consideration of IL-1 patterns. If so, it is possible that a previously “unsuccessful” clinical trial could be made “successful” when subjects are retroactively stratified by IL-1 pattern.

When subjects are stratified by IL-1 pattern, subjects of certain patterns who will benefit from the treatment are identified and subjects of other patterns who will not benefit (or benefit less) from the treatment are identified. Once the treatment is approved for clinical use, the stratified clinical trials will have revealed which patient populations (i.e., patients with a specific IL-1 pattern) should be provided the treatment and which patients should not.

### Ex Vivo Diagnostics

In aspects of the present invention, IL-1 levels can be measured ex vivo and in response to treatment with a therapeutic compound. For this, lymphocytes will be obtained from a subject. The lymphocytes will be treated with an IL-1 activator and then IL-1 levels (protein and/ or mRNA) will be measured. If the lymphocytes produce increased IL-1 and to a critical level, then a diagnosis of the subject can be made and a prediction regarding an optimal treatment can be determined.

### Isolated Nucleic Acid Molecules

As used herein, an “isolated nucleic acid molecule” generally is one that contains one or more of the SNPs disclosed herein or one that hybridizes to such molecule such as a nucleic acid with a complementary sequence, and is separated from most other nucleic acids present in the natural source of the nucleic acid molecule. As used herein, “a non-naturally occurring nucleic acid molecule” generally is one that contains one or more of the SNPs disclosed herein or one that hybridizes to such a molecule, such as a nucleic acid with a complementary sequence, but which does not correspond to a naturally occurring molecule, e.g., it can be a molecule prepared by recombinant nucleic acid technology, chemical synthesis, or other synthetic means such as polymerase chain reaction (PCR), and/ or a nucleic acid which comprises one or more synthetic components such as a non-natural nucleotide or an added tag/motif.

The isolated nucleic acid may be obtained from any bodily fluid (such as blood, serum, plasma, urine, saliva, phlegm, gastric juices, semen, tears, sweat, etc.), skin, hair, cell (especially nucleated cells), biopsy, buccal swab, tissue, or tumor specimen. Alternately, the isolated nucleic acid may be amplified or synthesized from a nucleic acid obtained from any bodily fluid, skin, hair, cell, biopsy, buccal swab, tissue, or tumor specimen.

Generally, an isolated SNP-containing nucleic acid molecule includes one or more of SNPs and/ or one or more SNPs in linkage disequilibrium with one or more SNPs. The isolated SNP-containing nucleic acid molecule may include flanking nucleotide sequences on either side of the SNP position. A flanking sequence can include nucleotide residues that are naturally associated with the SNP site and/ or heterologous nucleotide sequences. Preferably, the flanking sequence is up to about 10,000, 1,000, 500, 300, 100, 60, 50, 30, 25, 20, 15, 10, 8, or 4 nucleotides (or any other length in-between) on either side of a SNP position, or as long as the full-length gene, entire protein-coding sequence (or any portion thereof such as an exon), entire enhancer/promoter region or portion thereof, or entire intron or portion thereof.

An isolated SNP-containing nucleic acid molecule can include, for example, a full-length gene or transcript, such as a gene isolated from genomic DNA (e.g., by cloning or PCR amplification), a cDNA molecule, or an mRNA transcript molecule.

An isolated nucleic acid molecule of the disclosed subject matter further encompasses a SNP-containing polynucleotide that is the product of any one of a variety of nucleic acid amplification methods, which are used to increase the copy numbers of a polynucleotide of interest in a nucleic acid sample. Such amplification methods are well known in the art, and they include but are not limited to, polymerase chain reaction (PCR) (U.S. Pat. Nos. 4,683,195 and 4,683,202; PCR Technology: Principles and Applications for DNA Amplification, ed. H. A. Erlich, Freeman Press, NY, N.Y. (1992)), ligase chain reaction (LCR) (Wu and Wallace, Genomics 4:560 (1989); Landegren et al., Science 241:1077 (1988)), strand displacement amplification (SDA) (U.S. Pat. Nos. 5,270,184 and 5,422,252), transcription-mediated amplification (TMA) (U.S. Pat. No. 5,399,491), linked linear amplification (LLA) (U.S. Pat. No. 6,027,923) and the like, and isothermal amplification methods such as nucleic acid sequence based amplification (NASBA) and self-sustained sequence replication (Guatelli et al., Proc Natl Acad Sci USA 87:1874 (1990)). Based on such methodologies, a person skilled in the art can readily design primers in any suitable regions 5′ and 3′ to a SNP disclosed herein. Such primers may be used to amplify DNA of any length so long that it contains the SNP of interest in its sequence.

The isolated nucleic acid molecules that include, consist of, or consist essentially of one or more polynucleotide sequences that contain one or more SNPs disclosed herein, complements thereof, SNPs in linkage disequilibrium with the SNPs disclosed herein, and/or SNP-containing fragments thereof. Non-limiting examples of SNPs in linkage disequilibrium with the SNPs disclosed herein include those listed in Table 10 below.

| | | Linkage | |
|---|---|---|---|
| SNP | Common | SNP | Rsquared |
| rs16944 | B(−511) | rs1143627 | 0.965 |
| | | rs13013349 | 0.964 |
| | | rs1143623 | 0.827 |
| rs1143623 | B(−1464) | rs12621220 | 0.963 |
| | | rs1143627 | 0.864 |
| | | rs13008855 | 0.857 |
| | | rs16944 | 0.827 |
| | | rs12053091 | 0.824 |
| rs484306 | B(−3737) | None | |
| rs17561 | A(+4845) | rs3783557 | 0.961 |
| | | rs11898680 | 0.821 |
| | A(−889) | rs1800587 | |
| rs1143634 | B(+3954) | rs3917373 | 0.881 |

Isolated nucleic acid molecules can be in the form of RNA, such as mRNA, or in the form DNA, including cDNA and genomic DNA, which may be obtained, for example, by molecular cloning or produced by chemical synthetic techniques or by a combination thereof. Sambrook and Russell, Molecular Cloning: A Laboratory Manual, Cold Spring Harbor Press, N.Y. (2000). Furthermore, isolated nucleic acid molecules, particularly SNP detection reagents such as probes and primers, can also be partially or completely in the form of one or more types of nucleic acid analogs, such as peptide nucleic acid (PNA). U.S. Pat. Nos. 5,539,082; 5,527,675; 5,623,049; and 5,714,331. The nucleic acid, especially DNA, can be double-stranded or single-stranded. Single-stranded nucleic acid can be the coding strand (sense strand) or the complementary non-coding strand (anti-sense strand). DNA, RNA, or PNA segments can be assembled, for example, from fragments of the human genome (in the case of DNA or RNA) or single nucleotides, short oligonucleotide linkers, or from a series of oligonucleotides, to provide a synthetic nucleic acid molecule. Nucleic acid molecules can be readily synthesized using the sequences provided herein as a reference; oligonucleotide and PNA oligomer synthesis techniques are well known in the art. See, e.g., Corey, “Peptide nucleic acids: expanding the scope of nucleic acid recognition,” Trends Biotechnol 15 (6):224-9 (June 1997), and Hyrup et al., “Peptide nucleic acids (PNA): synthesis, properties and potential applications,” Bioorg Med Chem 4 (1):5-23 (January 1996). Furthermore, large-scale automated oligonucleotide/PNA synthesis (including synthesis on an array or bead surface or other solid support) can readily be accomplished using commercially available nucleic acid synthesizers, such as the Applied Biosystems (Foster City, Calif.) 3900 High-Throughput DNA Synthesizer or Expedite 8909 Nucleic Acid Synthesis System and the sequence information provided herein.

The isolated SNP-containing nucleic acid molecule may comprise modified, synthetic, or non-naturally occurring nucleotides or structural elements or other alternative/modified nucleic acid chemistries known in the art. Such nucleic acid analogs are useful, for example, as detection reagents (e.g., primers/probes) for detecting the SNPs identified herein. Furthermore, kits/systems (such as beads, arrays, etc.) that include these analogs are also encompassed herein.

The practice of the present methods will employ, unless otherwise indicated, conventional techniques of cell biology, cell culture, molecular biology, transgenic biology, microbiology, recombinant DNA, and immunology, which are within the skill of the art. Such techniques are explained fully in the literature. See, for example, Molecular Cloning: A Laboratory Manual, Cold Spring Harbor Laboratory (2001); DNA Cloning, Volumes I and II (P. N. Glover ed., 1985); Oligonucleotide Synthesis (M. J. Gait ed., 1984); Mullis et al. U.S. Pat. No. 4,683,195; Nucleic Acid Hybridization (B. D. Hames & S. J. Higgins eds. 1984); Transcription And Translation (B. Q. Hames & S. J. Higgins eds. 1984); Culture Of Animal Cells (R. I. Freshney, Alan R. Liss, Inc., 1987); Immobilized Cells And Enzymes (IRL Press, 1986); B. Perbal, A Practical Guide To Molecular Cloning (1984); the treatise, Methods In Enzymology (Academic Press, Inc., N.Y.); Gene Transfer Vectors For Mammalian Cells (J. H. Miller and M. P. Calos eds., 1987, Cold Spring Harbor Laboratory); Methods In Enzymology, Vols. 154 and 155 (Wu at al. eds.), Immunochemical Methods In Cell And Molecular Biology (Mayer and Walker, eds., Academic Press, London, 1987); Handbook Of Experimental Immunology, Volumes I-IV (D. M. Weir and C. C. Blackwell, eds., 1986); Manipulating the Mouse Embryo, (Cold Spring Harbor Laboratory Press, Cold Spring Harbor, N.Y., 1986).

### SNP Detection Reagents

In aspects of the present invention, each of the one or more of the SNPs disclosed herein can be used for the design of SNP detection reagents. As used herein, a “SNP detection reagent” is a reagent that specifically detects a specific target SNP position disclosed herein, and that is preferably specific for a particular nucleotide (allele) of the target SNP position (i.e., the detection reagent preferably can differentiate between different alternative nucleotides at a target SNP position, thereby allowing the identity of the nucleotide present at the target SNP position to be determined). Typically, such detection reagent hybridizes to a target SNP-containing nucleic acid molecule by complementary base-pairing in a sequence specific manner, and discriminates the target variant sequence from other nucleic acid sequences such as an art-known form in a test sample. An example of a detection reagent is a non-naturally occurring nucleic acid probe that hybridizes to a target nucleic acid containing one of the SNPs disclosed herein. In a preferred embodiment, such a probe can differentiate between nucleic acids having a particular nucleotide (allele) at the target SNP position from other nucleic acids that have a different nucleotide at the same target SNP position. In addition, a detection reagent may hybridize to a specific region 5′ and/ or 3′ to the SNP position.

Another example of a detection reagent is a non-naturally occurring nucleic acid primer that acts as an initiation point of nucleotide extension along a complementary strand of a target polynucleotide. The SNP sequence information provided herein is also useful for designing primers, e.g., allele-specific primers, to amplify (e.g., using PCR) the SNP of the disclosed subject matter.

A SNP detection reagent may be an isolated or synthetic DNA or RNA polynucleotide probe or primer or PNA oligomer, or a combination of DNA, RNA and/ or PNA that hybridizes to a segment of a target nucleic acid molecule containing one of the SNPs disclosed herein. A detection reagent in the form of a non-naturally occurring polynucleotide may optionally contain modified base analogs, intercalators, or minor groove binders. Multiple detection reagents such as probes may be, for example, affixed to a solid support (e.g., an array and bead) or supplied in solution (e.g., probe/primer sets for enzymatic reactions such as PCR, RT-PCR, TaqMan® assays, and primer-extension reactions) to form a SNP detection kit.

For analyzing SNPs, it can be appropriate to use oligonucleotides specific for alternative SNP alleles. Such oligonucleotides that detect single nucleotide variations in target sequences may be referred to by such terms as “allele-specific oligonucleotides,” “allele-specific probes,” or “allele-specific primers.” The design and use of allele-specific probes for analyzing polymorphisms is described in, e.g., Mutation Detection: A Practical Approach, Cotton et al., eds., Oxford University Press (1998); Saiki et al., Nature 324:163-166 (1986); Dattagupta, EP235,726; and Saiki, WO 89/11548.

In another embodiment, a probe or primer may be designed to hybridize to a segment of target DNA such that the SNP aligns with either the 5′-most end or the 3′-most end of the probe or primer. When using an oligonucleotide ligation assay (U.S. Pat. No. 4,988,617), the 3′ most nucleotide of the probe aligns with the SNP position in the target sequence.

Allele-specific probes are often used in pairs (or, less commonly, in sets of 3 or 4), and such pairs may be identical except for a one nucleotide mismatch that represents the allelic variants at the SNP position. Typically, one member of a probe pair perfectly matches a reference form of a target sequence that has a more common SNP allele (i.e., the allele that is more frequent in the target population) and the other member of the pair perfectly matches a form of the target sequence that has a less common SNP allele (i.e., the allele that is rarer in the target population). In the case of an array, multiple pairs of probes can be immobilized on the same support for simultaneous analysis of multiple different polymorphisms.

In one type of PCR-based assay, an allele-specific primer hybridizes to a region on a target nucleic acid molecule that overlaps a SNP position and only primes amplification of an allelic form to which the primer exhibits perfect complementarity. Gibbs, Nucleic Acid Res 17:2427-2448 (1989). Typically, the primer's 3′-most nucleotide is aligned with and complementary to the SNP position of the target nucleic acid molecule. This primer is used in conjunction with a second primer that hybridizes at a distal site. Amplification proceeds from the two primers, producing a detectable product that indicates which allelic form is present in the test sample. A control is usually performed with a second pair of primers, one of which shows a single base mismatch at the polymorphic site and the other of which exhibits perfect complementarity to a distal site. The single-base mismatch prevents amplification or substantially reduces amplification efficiency, so that either no detectable product is formed or it is formed in lower amounts or at a slower pace. The method generally works most effectively when the mismatch is at the 3′-most position of the oligonucleotide (i.e., the 3′-most position of the oligonucleotide aligns with the target SNP position) because this position is most destabilizing to elongation from the primer (see, e.g., WO 93/22456). This PCR-based assay can be utilized as part of the TaqMan® assay, described below.

A primer may contain a sequence substantially complementary to a segment of a target SNP-containing nucleic acid molecule except that the primer has a mismatched nucleotide in one of the three nucleotide positions at the 3′-most end of the primer, such that the mismatched nucleotide does not base pair with a particular allele at the SNP site. In a preferred embodiment, the mismatched nucleotide in the primer is the second from the last nucleotide at the 3′-most position of the primer. In a more preferred embodiment, the mismatched nucleotide in the primer is the last nucleotide at the 3′-most position of the primer.

A SNP detection reagent may be labeled with a fluorogenic reporter dye that emits a detectable signal. While the preferred reporter dye is a fluorescent dye, any reporter dye that can be attached to a detection reagent such as an oligonucleotide probe or primer is suitable for use in the disclosed subject matter. Such dyes include, but are not limited to, Acridine, AMCA, BODIPY, Cascade Blue, Cy2, Cy3, Cy5, Cy7, Dabcyl, Edans, Eosin, Erythrosin, Fluorescein, 6-Fam, Tet, Joe, Hex, Oregon Green, Rhodamine, Rhodol Green, Tamra, Rox, and Texas Red.

In yet another embodiment, the detection reagent may be further labeled with a quencher dye such as TAMRA, especially when the reagent is used as a self-quenching probe such as a TaqMan® (U.S. Pat. Nos. 5,210,015 and 5,538,848) or Molecular Beacon probe (U.S. Pat. Nos. 5,118,801 and 5,312,728), or other stemless or linear beacon probe (Livak et al., PCR Method Appl 4:357-362 (1995); Tyagi et al., Nature Biotechnology 14:303-308 (1996); Nazarenko et al., Nuc′ Acids Res 25:2516-2521 (1997); U.S. Pat. Nos. 5,866,336 and 6,117,635.

Detection reagents may also contain other labels, including but not limited to, biotin for streptavidin binding, hapten for antibody binding, and an oligonucleotide for binding to another complementary oligonucleotide.

Reagents may not contain (or be complementary to) a SNP nucleotide as describe herein but that are used to assay one or more SNPs disclosed herein. For example, primers that flank, but do not hybridize directly to a target SNP position provided herein are useful in primer extension reactions in which the primers hybridize to a region adjacent to the target SNP position (i.e., within one or more nucleotides from the target SNP site). During the primer extension reaction, a primer is typically not able to extend past a target SNP site if a particular nucleotide (allele) is present at that target SNP site, and the primer extension product can be detected in order to determine which SNP allele is present at the target SNP site. For example, particular ddNTPs are typically used in the primer extension reaction to terminate primer extension once a ddNTP is incorporated into the extension product (a primer extension product which includes a ddNTP at the Y-most end of the primer extension product, and in which the ddNTP is a nucleotide of a SNP disclosed herein, is a composition that is specifically herein). Thus, reagents that bind to a nucleic acid molecule in a region adjacent to a SNP site and that are used for assaying the SNP site, even though the bound sequences do not necessarily include the SNP site itself, are also contemplated by the disclosed subject matter.

For example, the SNP may be identified using single-base extension (SBE). SBE determines the identity of a nucleotide base at a specific position along a nucleic acid. In the method, an oligonucleotide primer hybridizes to a complementary region along the nucleic acid, to form a duplex, with the primer's terminal 3′ end directly adjacent to the nucleotide base to be identified. The oligonucleotide primer is enzymatically extended by a single base in the presence of all four nucleotide terminators; the nucleotide terminator complementary to the base in the template being interrogated is incorporated and identified. The presence of all four terminators ensures that no further extension occurs beyond the single incorporated base. Many approaches can be taken for determining the identity of a terminator, including fluorescence labeling, mass labeling for mass spectrometry, measuring enzyme activity using a protein moiety, and isotope labeling.

Reagents and techniques described herein may be directed to performance of “Next Generation Sequencing.” (See, e.g., Srivatsan et al., PLoS Genet 4: e100139 (2008); Rasmussen et al., Nature 463:757-762 (2010); Li et al., Nature 463: 311-317 (2010); Pelak et al., PLoS Genet 6: e1001111 (2010); Ram et al., Syst Biol Reprod Med (57(3):117-118 (2011); McEllistrem, Future Microbiol 4: 857-865 (2009); Lo et al., Clin Chem 55: 607-608 (2009); Robinson, Genome Biol 11:144 (2010); and Araya et al., Trends Biotechnology doi10. 1016.j.tibtech.2011.04.003 (2011)). For example, such techniques may involve the fragmentation of a genomic nucleic acid sample followed by parallel sequencing of those fragments and the alignment of the sequenced fragments to reconstruct the original sequence. Here, the genomic nucleic acid of interest is sheared into fragments and “adapters” (short nucleic acids of known sequence) are ligated to the fragments. Adaptor-modified fragments can be enriched via PCR. An adaptor-modified fragment (and amplified copies thereof, if present) may be flowed across a flow cell where the fragments are allowed to hybridize to primers immobilized on the surface of the cell. The fragments are then amplified by isothermal bridge amplification into a cluster consisting of thousands of molecules identical to the original. Sequencing primers can then be hybridized to the ends of one strand of the clusters, reversibly blocked, and labeled nucleotides added. The addition of each particular nucleotide can be identified by the label, then the label can be removed and the nucleotide un-blocked so that another blocked and labeled nucleotide can be added to identify the next position in the nucleic acid sequence. Once the desired number of rounds of addition, detection, and unblocking occur, the resulting sequences can be aligned.

It will be apparent to one of skill in the art that such primers and probes are directly useful as reagents for detecting the SNPs of the disclosed subject matter, and can be incorporated into any kit/system format.

### SNP Genotyping Methods

SNP genotyping includes, for example, collecting a biological sample from a human subject (e.g., sample of tissues, cells, fluids, secretions, etc.), isolating nucleic acids (e.g., genomic DNA, mRNA or both) from the cells of the sample, contacting the nucleic acids with one or more primers which specifically hybridize to a region of the isolated nucleic acid containing a target SNP under conditions such that hybridization and amplification of the target nucleic acid region occurs, and determining the nucleotide present at the SNP position of interest, or, in some assays, detecting the presence or absence of an amplification product (assays can be designed so that hybridization and/ or amplification will only occur if a particular SNP allele is present or absent). In some assays, the size of the amplification product is detected and compared to the length of a control sample; for example, deletions and insertions can be detected by a change in size of the amplified product compared to a normal genotype.

SNP genotyping is useful for numerous practical applications, as described herein. Examples of such applications include, but are not limited to, SNP-disease association analysis, disease predisposition screening, disease diagnosis, disease prognosis, disease progression monitoring, determining therapeutic strategies based on a subject's genotype (“pharmacogenomics”), developing therapeutic agents based on SNP genotypes associated with a disease or likelihood of responding to a drug, stratifying patient populations for clinical trials of a therapeutic, preventive, or diagnostic agent, and human identification applications such as forensics.

Nucleic acid samples can be genotyped to determine which allele is present at any given SNP position of interest by methods well known in the art. The neighboring sequence can be used to design SNP detection reagents such as oligonucleotide probes, which may optionally be implemented in a kit format. Exemplary SNP genotyping methods are described in Chen et al., “Single nucleotide polymorphism genotyping: biochemistry, protocol, cost and throughput,” Pharmacogenomics J 3 (2):77-96 (2003); Kwok et al., “Detection of single nucleotide polymorphisms,” Curr Issues Mol Biol 5 (2):43-60 (April 2003); Shi, “Technologies for individual genotyping: detection of genetic polymorphisms in drug targets and disease genes,” Am J Pharmacogenomics 2 (3):197-205 (2002); and Kwok, “Methods for genotyping single nucleotide polymorphisms,” Annu Rev Genom Hum Genet 2:235-58 (2001). Techniques for high-throughput SNP genotyping are described in Mamellos, “High-throughput SNP analysis for genetic association studies,” Curr Opin Drug Disc Devel 6 (3):317-21 (May 2003).

SNP genotyping methods include, but are not limited to, TaqMan® assays, molecular beacon assays, nucleic acid arrays, allele-specific primer extension, allele-specific PCR, arrayed primer extension, homogeneous primer extension assays, primer extension with detection by mass spectrometry, pyrosequencing, multiplex primer extension sorted on genetic arrays, ligation with rolling circle amplification, homogeneous ligation, Oligonucleotide Ligation Assay (OLA: U.S. Pat. No. 4,988,167), multiplex ligation reaction sorted on genetic arrays, restriction-fragment length polymorphism, single base extension-tag assays, denaturing gradient gel electrophoresis, and the Invader assay. Such methods may be used in combination with detection mechanisms such as, for example, luminescence or chemiluminescence detection, fluorescence detection, time-resolved fluorescence detection, fluorescence resonance energy transfer, fluorescence polarization, mass spectrometry, and electrical detection.

In one embodiment, SNP genotyping is performed using the TaqMan® assay, which is also known as the 5′ nuclease assay (U.S. Pat. Nos. 5,210,015 and 5,538,848). The TaqMan® assay detects the accumulation of a specific amplified product during PCR. The TaqMan® assay utilizes an oligonucleotide probe labeled with a fluorescent reporter dye and a quencher dye. The reporter dye is excited by irradiation at an appropriate wavelength, it transfers energy to the quencher dye in the same probe via a process called fluorescence resonance energy transfer (FRET). When attached to the probe, the excited reporter dye does not emit a signal. The proximity of the quencher dye to the reporter dye in the intact probe maintains a reduced fluorescence for the reporter. The reporter dye and quencher dye may be at the 5′ most and the 3′ most ends, respectively, or vice versa. Alternatively, the reporter dye may be at the 5′ or 3′ most end while the quencher dye is attached to an internal nucleotide, or vice versa. In yet another embodiment, both the reporter and the quencher may be attached to internal nucleotides at a distance from each other such that fluorescence of the reporter is reduced.

During PCR, the 5′ nuclease activity of DNA polymerase cleaves the probe, thereby separating the reporter dye and the quencher dye and resulting in increased fluorescence of the reporter. Accumulation of PCR product is detected directly by monitoring the increase in fluorescence of the reporter dye. The DNA polymerase cleaves the probe between the reporter dye and the quencher dye only if the probe hybridizes to the target SNP-containing template which is amplified during PCR, and the probe is designed to hybridize to the target SNP site only if a particular SNP allele is present.

Preferred TaqMan® primer and probe sequences can readily be determined using the SNP and associated nucleic acid sequence information provided herein. A number of computer programs, such as Primer Express (Applied Biosystems, Foster City, Calif.), can be used to rapidly obtain optimal primer/probe sets. These probes and primers can be readily incorporated into a kit format. The disclosed subject matter also includes modifications of the TaqMan® assay well known in the art such as the use of Molecular Beacon probes (U.S. Pat. Nos. 5,118,801 and 5,312,728) and other variant formats (U.S. Pat. Nos. 5,866,336 and 6,117,635).

Another method for genotyping the SNPs can be the use of two oligonucleotide probes in an OLA (see, e.g., U.S. Pat. No. 4,988,617). In this method, one probe hybridizes to a segment of a target nucleic acid with its 3′ most end aligned with the SNP site. A second probe hybridizes to an adjacent segment of the target nucleic acid molecule directly 3′ to the first probe. The two juxtaposed probes hybridize to the target nucleic acid molecule, and are ligated in the presence of a linking agent such as a ligase if there is perfect complementarity between the 3′ most nucleotide of the first probe with the SNP site. If there is a mismatch, ligation would not occur. After the reaction, the ligated probes are separated from the target nucleic acid molecule, and detected as indicators of the presence of a SNP.

The following patents, patent applications, and published international patent applications, which are all hereby incorporated by reference, provide additional information pertaining to techniques for carrying out various types of Oligonucleotide Ligation Assay (OLA). The following U.S. patents describe OLA strategies for performing SNP detection: U.S. Pat. Nos. 6,027,889; 6,268,148; 5,494,810; 5,830,711 and 6,054,564. WO 97/31256 and WO 00/56927 describe OLA strategies for performing SNP detection using universal arrays, where a zipcode sequence can be introduced into one of the hybridization probes, and the resulting product, or amplified product, hybridized to a universal zip code array. U.S. application Ser. No. 01/17,329 (and Ser. No. 09/584,905) describes OLA (or LDR) followed by PCR, where zipcodes are incorporated into OLA probes, and amplified PCR products are determined by electrophoretic or universal zipcode array readout. U.S. applications 60/427,818, 60/445,636, and 60/445,494 describe SNPIex methods and software for multiplexed SNP detection using OLA followed by PCR, where zipcodes are incorporated into OLA probes, and amplified PCR products are hybridized with a zipchute reagent, and the identity of the SNP determined from electrophoretic readout of the zipchute. In some embodiments, OLA is carried out prior to PCR (or another method of nucleic acid amplification). In other embodiments, PCR (or another method of nucleic acid amplification) is carried out prior to OLA.

Another method for SNP genotyping is based on mass spectrometry. Mass spectrometry takes advantage of the unique mass of each of the four nucleotides of DNA. SNPs can be unambiguously genotyped by mass spectrometry by measuring the differences in the mass of nucleic acids having alternative SNP alleles. MALDI-TOF (Matrix Assisted Laser Desorption Ionization-Time of Flight) mass spectrometry technology is preferred for extremely precise determinations of molecular mass, such as SNPs. Numerous approaches to SNP analysis have been developed based on mass spectrometry. Preferred mass spectrometry-based methods of SNP genotyping include primer extension assays, which can also be utilized in combination with other approaches, such as traditional gel-based formats and microarrays.

Typically, a mass spectrometry with primer extension assay involves designing and annealing a primer to a template PCR amplicon upstream (5′) from a target SNP position. A mix of dideoxynucleotide triphosphates (ddNTPs) and/ or deoxynucleotide triphosphates (dNTPs) are added to a reaction mixture containing template (e.g., a SNP-containing nucleic acid molecule which has typically been amplified, such as by PCR), primer, and DNA polymerase. Extension of the primer terminates at the first position in the template where a nucleotide complementary to one of the ddNTPs in the mix occurs. The primer can be either immediately adjacent (i.e., the nucleotide at the 3′ end of the primer hybridizes to the nucleotide next to the target SNP site) or two or more nucleotides removed from the SNP position. If the primer is several nucleotides removed from the target SNP position, the only limitation is that the template sequence between the 3′ end of the primer and the SNP position cannot contain a nucleotide of the same type as the one to be detected, or this will cause premature termination of the extension primer. Alternatively, if all four ddNTPs alone, with no dNTPs, are added to the reaction mixture, the primer will always be extended by only one nucleotide, corresponding to the target SNP position. In this instance, primers are designed to bind one nucleotide upstream from the SNP position (i.e., the nucleotide at the 3′ end of the primer hybridizes to the nucleotide that is immediately adjacent to the target SNP site on the 5′ side of the target SNP site). Extension by only one nucleotide is preferable, as it minimizes the overall mass of the extended primer, thereby increasing the resolution of mass differences between alternative SNP nucleotides. Furthermore, mass-tagged ddNTPs can be employed in the primer extension reactions in place of unmodified ddNTPs. This increases the mass difference between primers extended with these ddNTPs, thereby providing increased sensitivity and accuracy, and is particularly useful for typing heterozygous base positions.

Primer extension assays may be used in conjunction with MALDI-TOF mass spectrometry for SNP genotyping, see, e.g., Wise et al., “A standard protocol for single nucleotide primer extension in the human genome using matrix-assisted laser desorption/ionization time-of-flight mass spectrometry,” Rapid Comm. Mass Spect. 17 (11):1195-202 (2003).

SNPs can also be scored by direct DNA sequencing. A variety of automated sequencing procedures can be utilized (e.g., Biotechniques 19:448 (1995)), including sequencing by mass spectrometry. See, e.g., PCT International Publication No. WO 94/16101; Cohen et al., Adv Chromatogr 36:127-162 (1996); and Griffin et al, Appl Biochem Biotechnol 38:147-159 (1993). The nucleic acid sequences of the disclosed subject matter enable one of ordinary skill in the art to readily design sequencing primers for such automated sequencing procedures. Commercial instrumentation, such as the Applied Biosystems 377, 3100, 3700, 3730, and 3730×1 DNA Analyzers (Foster City, Calif.), is commonly used in the art for automated sequencing.

Other methods that can be used to genotype the SNPs of the disclosed subject matter include single-strand conformational polymorphism (SSCP), and denaturing gradient gel electrophoresis (DGGE). Myers et al., Nature 313:495 (1985). SSCP identifies base differences by alteration in electrophoretic migration of single stranded PCR products, as described in Orita et al., Proc. Nat. Acad. Single-stranded PCR products can be generated by heating or otherwise denaturing double stranded PCR products. Single-stranded nucleic acids may refold or form secondary structures that are partially dependent on the base sequence. The different electrophoretic mobilities of single-stranded amplification products are related to base-sequence differences at SNP positions. DGGE differentiates SNP alleles based on the different sequence-dependent stabilities and melting properties inherent in polymorphic DNA and the corresponding differences in electrophoretic migration patterns in a denaturing gradient gel. PCR Technology: Principles and Applications for DNA Amplification Chapter 7, Erlich, ed., W.H. Freeman and Co, N.Y. (1992).

Sequence-specific ribozymes (U.S. Pat. No. 5,498,531) can also be used to score SNPs based on the development or loss of a ribozyme cleavage site. Perfectly matched sequences can be distinguished from mismatched sequences by nuclease cleavage digestion assays or by differences in melting temperature. If the SNP affects a restriction enzyme cleavage site, the SNP can be identified by alterations in restriction enzyme digestion patterns, and the corresponding changes in nucleic acid fragment lengths determined by gel electrophoresis.

### SNP Detection Kits and Systems

A person skilled in the art will recognize that, based on the SNP and associated sequence information disclosed herein, detection reagents can be developed and used to assay the SNP of the disclosed subject matter individually or in combination with other SNPs, and such detection reagents can be readily incorporated into one of the established kit or system formats which are well known in the art.

The terms “kits” and “systems,” as used herein in the context of SNP detection reagents, are intended to refer to such things as combinations of multiple SNP detection reagents, or one or more SNP detection reagents in combination with one or more other types of elements or components (e.g., other types of biochemical reagents, containers, packages such as packaging intended for commercial sale, substrates to which SNP detection reagents are attached, electronic hardware components, and software recorded on a non-transitory processor-readable medium). Accordingly, the disclosed subject matter further provides SNP detection kits and systems, including but not limited to, packaged probe and primer sets (e.g., TaqMan® probe/primer sets), arrays/microarrays of nucleic acid molecules, and beads that contain one or more probes, primers, or other detection reagents for detecting one or more SNPs of the disclosed subject matter.

The kits/systems can optionally include various electronic hardware components; for example, arrays (“DNA chips”) and microfluidic systems (“lab-on-a-chip” systems) provided by various manufacturers typically include hardware components. Other kits/systems (e.g., probe/primer sets) may not include electronic hardware components, but may include, for example, one or more SNP detection reagents (along with, optionally, other biochemical reagents) packaged in one or more containers.

In some embodiments, a SNP detection kit typically contains one or more detection reagents and other components (e.g., a buffer, enzymes such as DNA polymerases or ligases, chain extension nucleotides such as deoxynucleotide triphosphates, and in the case of Sanger-type DNA sequencing reactions, chain terminating nucleotides, positive control sequences, negative control sequences, and the like) necessary to carry out an assay or reaction, such as amplification and/ or detection of a SNP-containing nucleic acid molecule.

A kit may further contain instructions for using the kit to detect the SNP-containing nucleic acid molecule of interest.

The instructions may include information which allows a user to identify whether a subject having or suspected of having an inflammation-related cancer or cancer risk has genotype-specific differential expression of IL-1, i.e., is a “high” or “low” producer of IL-1, based upon the composite IL-1 genotype or IL-1 genotype patterns disclosed in Tables 1-3. The instructions may include information which allows a user to decide on an appropriate inflammation inhibitor (e.g., as disclosed in Table 4 and/ or an alternate inhibitor having a similar or identical mode of action as an agent disclosed in Table 4) and at an appropriate dose.

In one embodiment, kits are provided which contain the necessary reagents to carry out one or more assays to detect one or more SNPs disclosed herein. In another embodiment, SNP detection kits/systems are in the form of nucleic acid arrays, or compartmentalized kits, including microfluidic/lab-on-a-chip systems.

SNP detection kits/systems may contain, for example, one or more probes, or pairs of probes, that hybridize to a nucleic acid molecule at or near each target SNP position. Multiple pairs of allele-specific probes may be included in the kit/system to simultaneously assay large numbers of SNPs, at least one of which is the SNP of the disclosed subject matter. In some kits/systems, the allele-specific probes are immobilized to a substrate such as an array or bead.

The terms “arrays,” “microarrays,” and “DNA chips” are used herein interchangeably to refer to an array of distinct polynucleotides affixed to a substrate, such as glass, plastic, paper, nylon or other type of membrane, filter, chip, or any other suitable solid support. The polynucleotides can be synthesized directly on the substrate, or synthesized separate from the substrate and then affixed to the substrate.

Any number of probes, such as allele-specific probes, may be implemented in an array, and each probe or pair of probes can hybridize to a different SNP position. In the case of polynucleotide probes, they can be synthesized at designated areas (or synthesized separately and then affixed to designated areas) on a substrate using a light-directed chemical process. Each DNA chip can contain, for example, thousands to millions of individual synthetic polynucleotide probes arranged in a grid-like pattern and miniaturized (e.g., to the size of a dime). Preferably, probes are attached to a solid support in an ordered, addressable array.

A SNP detection kit/system can include components that are used to prepare nucleic acids from a test sample for the subsequent amplification and/ or detection of a SNP-containing nucleic acid molecule. Such sample preparation components can be used to produce nucleic acid extracts (including DNA and/ or RNA), proteins or membrane extracts from any bodily fluids (such as blood, serum, plasma, urine, saliva, phlegm, gastric juices, semen, tears, sweat, etc.), skin, hair, cells (especially nucleated cells), biopsies, buccal swabs or tissue or tumor specimens. Methods of preparing nucleic acids, proteins, and cell extracts are well known in the art and can be readily adapted to obtain a sample that is compatible with the system utilized. Automated sample preparation systems for extracting nucleic acids from a test sample are commercially available, and examples are Qiagen's BioRobot 9600, Applied Biosystems' PRISM 6700 sample preparation system, and Roche Molecular Systems' COBAS AmpliPrep System.

For genotyping SNPs, an exemplary microfluidic system may integrate, for example, nucleic acid amplification, primer extension, capillary electrophoresis, and a detection method such as laser induced fluorescence detection. In an exemplary process for using such an exemplary system, nucleic acid samples are amplified, preferably by PCR. Then, the amplification products are subjected to automated primer extension reactions using ddNTPs (specific fluorescence for each ddNTP) and the appropriate oligonucleotide primers to carry out primer extension reactions which hybridize just upstream of the targeted SNP. Once the extension at the 3′ end is completed, the primers are separated from the unincorporated fluorescent ddNTPs by capillary electrophoresis. The separation medium used in capillary electrophoresis can be, for example, polyacrylamide, polyethyleneglycol or dextran. The incorporated ddNTPs in the single nucleotide primer extension products are identified by laser-induced fluorescence detection. Such an exemplary microchip can be used to process, for example, at least 96 to 384 samples, or more, in parallel.

An exemplary kit allows a user to determine whether a subject has genotype-specific differential expression of IL-1, i.e., is a “high” or “low” producer of IL-1, based upon the composite IL-1 genotype or IL-1 genotype patterns disclosed in Tables 1-3 and has a relevant status of one or more risk factors, as disclosed herein. The exemplary kit may include instructions having information which allows a user to decide on an appropriate agent or agents for IL-1 based treatment (e.g., as disclosed in Table 4 and/ or an alternate agents(s) having a similar or identical mode of action as those disclosed in Table 4) and at an appropriate dose.

### Reports, Programmed Computers, and Systems

The results of a test provide an identification of a composite IL-1 genotype or IL-1 genotype pattern, as disclosed in Tables 1-3 and identification of the status for one or more risk factors, as disclosed herein, which together determine a subject's predicted responsiveness to an inflammation inhibiting agent (e.g., a response to an agent disclose in Table 4 and/ or an alternate agent having a mode of action similar to or identical to an agent from Table 4). The results may be referred to herein as a “report”. The report may include other information based on assaying the SNPs disclosed herein, alone or in combination with other SNPs, and/ or a subject's allele/genotype at the SNPs disclosed herein, alone or in combination with other SNPs, etc.), and/ or any other information pertaining to a test.

A tangible report can optionally be generated as part of a testing process (which may be interchangeably referred to herein as “reporting”, or as “providing” a report, “producing” a report, or “generating” a report).

Examples of tangible reports may include, but are not limited to, reports in paper (such as computer-generated printouts of test results or hand written reports) or equivalent formats and reports stored on computer readable medium (such as a CD, USB flash drive or other removable storage device, computer hard drive, or computer network server, etc.). Reports, particularly those stored on computer readable medium, can be part of a database, which may optionally be accessible via the internet (such as a database of patient records or genetic information stored on a computer network server, which may be a “secure database” that has security features that limit access to the report, such as to allow only the patient and the patient's medical practitioners to view the report while preventing other unauthorized subjects from viewing the report, for example). In addition to, or as an alternative to, generating a tangible report, reports can also be displayed on a computer screen (or the display of another electronic device or instrument).

In addition to, or as an alternative to, the report may be “intangible” in that it is orally presented to another.

A tangible report may be hand written or may be prepared using a computer.

A report may be provided to the subject who can then implement the information and/ or instructions contained therein.

A report may be provided to a health care professional who can then implement the information and/ or instructions contained therein and/ or instruct the subject (e.g., prescribe and make a recommendation).

A report can include, for example, a subject's predicted drug responsiveness (e.g., to an agent disclosed in Table 4 and/ or an alternate agent having a mode of action similar to or identical to an agent from Table 4 based upon his/her composite IL-1 genotype or IL-1 genotype pattern, as disclosed in Tables 1-3 and status of one or more clinical indicators, as disclosed herein; the allele/genotype that a subject carries at the SNP locations disclosed herein; the status of his/her clinical indicators; and/ or his/her composite IL-1 genotype or IL-1 genotype pattern. Thus, for example, the report can include information of medical/biological significance (e.g., drug responsiveness, suggested treatment, and prophylactic methods). The report may just include allele/genotype information and/ or a composite IL-1 genotype or IL-1 genotype pattern and status of one or more clinical indicators but without including disease risk or other medical/biological significance; thus, the subject viewing the report can use the allele/genotype information and/ or composite IL-1 genotype or IL-1 genotype pattern and status of one or more clinical indicators to determine the associated disease risk or other medical/biological significance from a source outside of the report itself, such as from a medical practitioner, publication, website, etc., which may optionally be linked to the report such as by a hyperlink.

A report can further be “transmitted” or “communicated” (these terms may be used herein interchangeably), such as to the subject who was tested, a medical practitioner (e.g., a doctor, nurse, clinical laboratory practitioner, genetic counselor, etc.), a healthcare organization, a clinical laboratory, and/ or any other party or requester intended to view or possess the report. The act of “transmitting” or “communicating” a report can be by any means known in the art, based on the format of the report. Furthermore, “transmitting” or “communicating” a report can include delivering a report (“pushing”) and/ or retrieving (“pulling”) a report. For example, reports can be transmitted/communicated by various means, including being physically transferred between parties (such as for reports in paper format) such as by being physically delivered from one party to another, or by being transmitted electronically or in signal form (e.g., via e-mail or over the internet, by facsimile, and/ or by any wired or wireless communication methods known in the art) such as by being retrieved from a database stored on a computer network server.

Additional teaching relevant to the present invention are described in one or more of the following: U.S. Pat. Nos. 5,686,246, 5,698,399, 5,808,918, 6,108,635, 6,140,047, 6,210,877, 6,251,598, 6,268,142, 6,383,775, 6,437,216, 6,524,795, 6,551,785, 6,558,905, 6,706,478, 6,713,253, 6,720,141, 6,730,476, 6,733,967, 6,746,839, 7,723,028, 7,820,383, 8,101,360, 8,105,775, US 2002/0182612, US 2003/0100031, US 2003/0124524, US 2003/0152947, US 2003/0235890, US 2004/0152124, US 2005/0032077, US 2005/0064453, US 2005/0171338, US 2005/0282198, US 2006/0183161, US 2006/0252050, US 2007/0264645, US 2007/0275104, US 2008/0118920, US 2008/0187920, US 2008/0199865, US 2008/0254476, US 2008/0254477, US 2008/0254478, US 2008/0311581, US 2009/0023147, US 2009/0093396, US 2009/0163460, US 2009/0170105, US 2009/0191564, US 2010/0028893, US 2010/0129798, US 2010/0255475, US 2010/0279280, US 2011/0008906, US 2013/0011841, US 2003/0175764, US 2004/0110168, US 2010/0098775, US 2010/0098809, US 2010/0105038, US 2010/0112570, US 2010/0136561, US 2012/0208187 and US 2013/0337448, each of which is incorporated herein by reference in their entireties.

The term “single nucleotide polymorphisms” (SNPs) refers to a variation in the sequence of a gene in the genome of a population that arises as the result of a single base change, such as an insertion, deletion or, a change in a single base. A locus is the site at which divergence occurs. SNPs can result in modified amino acid sequences, altering structure and function of coded protein, and influence the splicing process when present at exon-intron transitions and modify gene transcription when part of promoters. This modification can lead to altered levels of protein expression.

As used herein the term subject is meant to include any human subject. A subject may be less than 60 years old. The subject may have one or more risk factors for lung cancer, or have been diagnosed with lung cancer.

As used herein, the terms “drug”, “medication”, “therapeutic”, “active agent”, “therapeutic compound”, “composition”, or “compound” are used interchangeably and refer to any chemical entity, pharmaceutical, drug, biological, botanical, and the like that can be used to treat or prevent a disease, illness, condition, or disorder of bodily function. A drug may comprise both known and potentially therapeutic compounds. A drug may be determined to be therapeutic by screening using the screening known to those having ordinary skill in the art. A “known therapeutic compound”, “drug”, or “medication” refers to a therapeutic compound that has been shown (e.g., through animal trials or prior experience with administration to humans) to be effective in such treatment. A “therapeutic regimen” relates to a treatment comprising a “drug”, “medication”, “therapeutic”, “active agent”, “therapeutic compound”, “composition”, or “compound” as disclosed herein and/ or a treatment comprising behavioral modification by the subject and/ or a treatment comprising a surgical means.

All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting.

Although methods and materials similar or equivalent to those described herein can be used in the practice or testing of the present invention, suitable methods and materials are described below. All publications, patent applications, patents, and other references mentioned herein are incorporated by reference in their entirety. The references cited herein are not admitted to be prior art to the claimed invention. In the case of conflict, the present Specification, including definitions, will control. In addition, the materials, methods, and examples are illustrative only and are not intended to be limiting.

Unless otherwise defined, all technical and scientific terms used herein have the same meaning as commonly understood by one of ordinary skill in the art to which this application belongs and as commonly used in the art to which this application belongs; such art is incorporated by reference in its entirety.

Any of the above aspects and embodiments can be combined with any other aspect or embodiment as disclosed in the Summary, and/ or in the Detailed Description sections.

### REFERENCES

Ridker P. M., Buring J. E, Cook N. R., Rifai N. C-reactive protein, the metabolic syndrome, and risk of incident cardiovascular events: an 8-year follow-up of 14 719 initially healthy american women. Circulation 2003; 107:391-397.
Ridker P. M, Danielson E, Fonseca F. A, et al. Rosuvastatin to prevent vascular events in men and women with elevated c-reactive protein. The New England Journal of Medicine 2008; 359:2195-2207.
Ridker P. M., Hennekens C. H., Buring J. E., Rifai n. C-reactive protein and other markers of inflammation in the prediction of cardiovascular disease in women. The New England Journal of Medicine 2000; 342:836-843.
Netea M. G., Balkwill F, Chonchol M, et al. A guiding map for inflammation. Nat Immunol 2017; 18:826-831.
Ridker P. M., Everett B. M., Thuren T., et al. Antiinflammatory therapy with canakinumab for atherosclerotic disease. The New England Journal of Medicine 2017; 377:1119-1131.
Ridker P. M., MacFadyen J. G., Everett B. M., et al. Relationship of c-reactive protein reduction to cardiovascular event reduction following treatment with canakinumab: a secondary analysis from the CANTOS randomised controlled trial. Lancet 2018 (391):319-328.
Ridker P. M., MacFadyen J. G., Thuren T, et al. Effect of interleukin-1beta inhibition with canakinumab on incident lung cancer in patients with atherosclerosis: exploratory results from a randomised, double-blind, placebo-controlled trial. Lancet 2017; 390:1833-1842.
Ridker P M, MacFadyen J G, Thuren T, Libby P. Residual inflammatory risk associated with interleukin-18 and interleukin-6 after successful interleukin-1beta inhibition with canakinumab: further rationale for the development of targeted anti-cytokine therapies for the treatment of atherothrombosis. Eur Heart J 2019.
Ridker P M. Anti-inflammatory therapy for atherosclerosis: interpreting divergent results from the CANTOS and CIRT clinical trials. J Intern Med 2019; 285:503-9.
Aday A W, Ridker P M. Targeting Residual Inflammatory Risk: A Shifting Paradigm for Atherosclerotic Disease. Front Cardiovasc Med 2019; 6:16.
Aggarwal B B, Shishodia S, Sandur S K, Pandey M K, Sethi G. (2006) Inflammation and cancer: how hot is the link? Biochem Pharmacol, 72:1605-21.
Aggarwal B B, Vijayalekshmi R V, Sung B. (2009) Targeting inflammatory pathways for prevention and therapy of cancer: Short-term friend, long-term foe. Clin Cancer Res, 15: 425-430.
Apte R N, Dvorkin T, Song X, Fima E, et al. (2000) Opposing effects of IL-1 alpha and IL-1 beta on malignancy patterns. Tumor cell-associated IL-1 alpha potentiates anti-tumor immune responses and tumor regression, whereas IL-1 beta potentiates invasiveness. Adv. Exp. Med. Biol. 479, 277-288.
Balkwill F R, Mantovani A. (2012) Cancer-related inflammation: common themes and therapeutic opportunities. Semin. Cancer Biol. 22:33-40.
Bani, M. R.; Garofalo, A.; Scanziani, E.; Giavazzi, R. (1991) Effect of interleukin-1-beta on metastasis formation in different tumor systems. J. Natl. Cancer Ins., 83(2):119-123.
Basu A, Krady J K, Levison S W (2004) Interleukin-1: a master regulator of neuroinflammation. J Neurosci Res, 78: 151-156.
Berse, B, Hunt J A, Diegel R J, Morganelli P, Yeo K, Brown F, et al. (1999). Hypoxia augments cytokine (transforming growth factor-beta (TGFbeta) and IL-1)-induced vascular endothelial growth factor secretion by human synovial fibroblasts. Clin. Exp. Immunol, 115, 176-182. doi: 10.1046/j.1365-2249.1999.00775.x
Berger P, McConnell J P, Nunn M, et al. C-reactive protein levels are influenced by common IL-1 gene variations. Cytokine 2002; 17:171-174.
Carmi, Y., Voronov, E., Dotan, S., Lahat, N., Rahat, M. A., Fogel, M., et al. (2009). The role of macrophage-derived IL-1 in induction and maintenance of angiogenesis. J Immunol, 183, 4705-4714.
Chen H, Wilkins L M, Aziz N, et al. Single nucleotide polymorphisms in the human interleukin-1B gene affect transcription according to haplotype context. Hum Mol Genet 2006; 15:519-529.s
Coussens L M, and Werb Z. (2002) Inflammation and cancer. Nature, 420: 60-867.
Dinarello C A (1996) Biologic basis for interleukin-1 in disease. Blood, 87(6): 2095-2147.
Dinarello C A. (2014) Retrospective Mol Med, 20 (SUPPLEMENT 1): S43-S58.
Farrow B and Evers B M. (2002) Inflammation and the development of pancreatic cancer. Surg Oncol, 10:153-169.
Federico A, Morgillo F, Tuccillo C, Ciardiello F, Loguercio C. (2007) Chronic inflammation and oxidative stress in human carcinogenesis. Int. J. Cancer, 121:2381-86.
Fernandes J V, Fernandes T A A, De Azevedo J C V, Cobucci R N O, et al. (2015) Link between chronic inflammation and human papillomavirus-induced carcinogenesis (Review). Oncology Letters, 9: 1015-1026.
Grivennikov S I, Greten F R, Karin M (2010) Immunity, inflammation, and cancer. Cell 140: 883-899.
He Q, Fu Y, Tian D, Yan W. (2018) The contrasting roles of inflammasomes in cancer. Am J Cancer Res, 8(4):566-583.
Hussain S P and Harris C C. (2007) Inflammation and cancer: An ancient link with novel potentials. Int. J. Cancer, 121:2373-80.
Jeng J H, Wang Y J, Chiang B L, Lee P H, Chan C P, et al. Roles of keratinocyte inflammation in oral cancer: Regulating the prostaglandin E2, interleukin-6 and TNF-alpha production of oral epithelial cells by areca nut extract and arecoline. Carcinogenesis, 24:1301-1315.
Jung Y J1, Isaacs J S, Lee S, Trepel J, Neckers L. (2003) IL-1beta-mediated up-regulation of HIF-1alpha via an NFkappaB/COX-2 pathway identifies HIF-1 as a critical link between inflammation and oncogenesis. FASEB j, 17(14):2115-17.
Kiyohara, C, Horiuchi T, Takayama K, Nakanishi Y. (2010) IL10 rs1143634 Polymorphism, Cigarette Smoking, Alcohol Use, and Lung Cancer Risk in a Japanese Population. J Thorac Oncol, 5:299-304.
Kinzler K W and Vogelstein B. (1998) Landscaping the cancer terrain. Science 280:1036-1037.
Krelin Y, et al. (2007) Interleukin-1beta-driven inflammation promotes the development and invasiveness of chemical carcinogen-induced tumors. Cancer Res, 67:1062-71.
Kurtzman S H, Anderson K H, Wang Y et al. (1999) Cytokines in human breast cancer: IL-1alpha and IL-1beta expression. Oncol Rep, 6: 65-70.
Landvik N E, Hart K, Haugen A, Zienolddiny S. Functional analysis of a lung cancer risk haplotype in the IL1B gene regulatory region. J Hum Genet 2012; 57:747-752.
Landvik N E, Hart K, Skaug V, Stangeland L B, Haugen A, Zienolddiny S. A specific interleukin-1B haplotype correlates with high levels of IL1B mRNA in the lung and increased risk of non-small cell lung cancer. Carcinogenesis 2009; 30:1186-1192.
Lee K M, Park S K, Hamajima N, Tajima K, et al (2006) Genetic polymorphisms of interleukin-1 beta (IL-1B) and IL-1 receptor antagonist (IL-1RN) and breast cancer risk in Korean women. Breast Cancer Res Treat, 96(2):197-202.
Lee Ch, Chang J S M, Syu S H, Wong T S. (2015) IL-1β Promotes Malignant Transformation and Tumor Aggressiveness in Oral Cancer. J Cell Physiol, 230:875-884.
Li Y, Wang L, Pappan L, Galliher-Beckley A, Shi J. (2012) IL-1β promotes sternness and invasiveness of colon cancer cells through Zeb1 activation. Mol Cancer, 11:87-99.
Liss, C.; Fekete, M. J.; Hasina, R.; Lam, C. D.; Lingen, M. W. (2001) Paracrine angiogenic loop between head-and-neck squamous-cell carcinomas and macrophages. Int. J. Cancer, 93(6):781-785.
Liu Q, Russell M R, Shahriari K et al. (2013) Interleukin-1beta promotes skeletal colonization and progression of metastatic prostate cancer cells with neuroendocrine features. Cancer Res, 73: 3297-305.
Lust J A, Donovan K A. (1999) The role of interleukin-1 beta in the pathogenesis of multiple myeloma. Hematol. Oncol. Clin. North Am. 13:1117-25.
Lust J A, et al. (2009) Induction of a chronic disease state in patients with smoldering or indolent multiple myeloma by targeting interleukin 1β-induced interleukin 6 production and the myeloma proliferative component. Mayo Clin. Proc. 84:114-22.
Maruyama, K., Mori, Y., Murasawa, S., Masaki, H., Takahashi, N., Tsutusmi, Y., et al. (1999). Interleukin-1 beta upregulates cardiac expression of vascular endothelial growth factor and its receptor KDR/flk-1 via activation of protein tyrosine kinases. J. Mol. Cell. Cardiol, 31:607-617.
Michaud, D. S. et al. (2018) Periodontal disease assessed using clinical dental measurements and cancer risk in the ARIC study. JNCI J Natl Cancer Inst 110(8): djx278.
Mitsunaga S, Ikeda M, Shimizu S, Ohno I, Furuse J, et al. (2013) Serum levels of IL-6 and IL-1β can predict the efficacy of gemcitabine in patients with advanced pancreatic cancer. Br J Cancer, 108(10):2063-9.
Mohr T, Haudek-Prinz V, Slany A, Grillari J, Micksche M, Gerner C (2017) Proteome profiling in IL-1β and VEGF-activated human umbilical vein endothelial cells delineates the interlink between inflammation and angiogenesis. PLoS ONE, 12(6):e0179065.
Nadini A and Carraro F. (2005) Role of Inflammatory Mediators in Angiogenesis. Curr Drug Targ-Inflamm & Allergy, 4:3-8.
Nasu, K., Itoh, H., Yuge, A., Kawano, Y., Yoshimatsu, J., and Narahara, H. (2006). Interleukin-1beta regulates vascular endothelial growth factor and soluble fmslike tyrosine kinase-1 secretion by human oviductal epithelial cells and stromal fibroblasts. Gynecol. Endocrinol, 22:495-500.
Nutter F, Holen I, Brown H K, Cross S S, Evans C A, Walker M, Coleman R E, Westbrook J A, Selby P J, Brown J E, et al. (2014) Different molecular profiles are associated with breast cancer cell homing compared with colonisation of bone: evidence using a novel bone-seeking cell line. Endocr Relat Cancer, 21:327-341.
Ohshima, M. Tatemichi, and T. Sawa. (2003) Chemical basis of inflammation-induced carcinogenesis. Archives of Biochemistry and Biophysics, 417(1): 3-11.
Ohnishi S, Ma N, Thanan R, Pinlaor S, et al. (2013) DNA Damage in Inflammation-Related Carcinogenesis and Cancer Stem Cells. Oxid Med Cell Longev, 2013:387014. doi: 10.1155/2013/3870.
Ozbabacan SEA, Gursoy A, Nussinov R, Keskin O. (2014) The Structural Pathway of Interleukin 1 (IL-1) Initiated Signaling Reveals Mechanisms of Oncogenic Mutations and SNPs in Inflammation and Cancer. PLoS Comput Biol 10(2): e1003470. doi:10.1371/journal.pcbi.1003470.
Peleteiro B, Lunet N, Carrilho C, Durães C, et al. (2010) Association Between Cytokine Gene Polymorphisms and Gastric Precancerous Lesions: Systematic Review and Meta-analysis. Cancer Epidemiol Biomarkers Prev, 19(3):62-76.
Rao S K, Pavicevic Z, Du Z, Kim J G, Fan M, Jiao Y, Rosebush M, Samant S, Gu W, Pfeffer L M, Nosrat C A. (2010) Pro-inflammatory genes as biomarkers and therapeutic targets in oral squamous cell carcinoma. J Biol Chem, 285:32512-21.
Rogus J, Beck J D, Offenbacher S, et al. IL1B gene promoter haplotype pairs predict clinical levels of interleukin-1beta and C-reactive protein. Hum Genet 2008; 123:387-398.
Sanabria-Salas M C, Hernandez-Suarez G, Umana-Perez A, et al. IL1B-CGTC haplotype is associated with colorectal cancer in admixed subjects with increased African ancestry. Sci Rep 2017; 7:41920.
Schneider S, Ross A, and Grichnik J M. (2014) Do inflammatory pathways drive melanomagenesis? Experiment Dermat, 24:86-90.
Song Z, Yun Lin, Ye X, Feng C, Lu Y, et al. (2016) IL-1a and IL-6 expression in cervical cancer. Med Sci Monit, 22: 4475-4481.
Stavri G T, Zachary I C, Baskerville P A, Martin, J F, and Erusalimsky J D. (1995). Basic fibroblast growth factor upregulates the expression of vascular endothelial growth factor in vascular smooth muscle cells. Synergistic interaction with hypoxia. Circulation, 92:11-14.
Takahashi H, Ogata H, Nishigaki R, Broide D H, Michael Karin M. (2009) Tobacco Smoke Promotes Lung Tumorigenesis by Triggering IKKβ and JNK1-Dependent Inflammation. Cancer Cell, 17:89-97.
Tang R F, Wang S X, Zhang F R et al: Interleukin-1alpha, 6 regulate the secretion of vascular endothelial growth factor A, C in pancreatic cancer. Hepatobiliary Pancreat Dis Int, 4: 460-63.
Tarassishin L, Lim J, Weatherly D B, Angeletti R H, Lee S C (2014a) Interleukin-1-induced changes in the glioblastoma secretome suggest its role in tumor progression. J Proteomics, 99C: 152-168.
Tarassishin L, Casper D, Lee S C (2014b) Aberrant Expression of Interleukin-1b and Inflammasome Activation in Human Malignant Gliomas. PLoS ONE, 9(7): e103432. doi:10.1371/journal.pone.0103432.
Thornton P, Pinteaux E, Gibson R M, Allan S M, Rothwell N J (2006) Interleukin-1-induced neurotoxicity is mediated by glia and requires caspase activation and free radical release. J Neurochem, 98: 258-266.
Vincenzi B, Patti G, Galluzzo S, Pantano F, et al. (2008) Interleukin 1β-511T gene (IL1β) polymorphism is correlated with gastric cancer in the Caucasian population: Results from a meta-analysis. Oncology Reports, 20: 1213-1220.
Voronov E, Carmi Y, Apte R N. (2014) The role of IL-1intumor-mediated angiogenesis. Frontiers Physiol, 5: doi: 10.3389/fphys.2014.00114.
Wang L, Liu Z, Li Y et al. (2012) Pro-inflammatory cytokine interleukin-10 promotes the development of intestinal stem cells. Inflamm Res, 61:1085-1092.
Wang L, Zhang L F, Wu J, Xu S J, Xu Y Y, et al. (2014a) IL-1b-Mediated Repression of microRNA-101 Is Crucial for Inflammation-Promoted Lung Tumorigenesis. Cancer Res, 74(17): 4720-30.
Wang Y, Wang K, Han G C, Wang R X, et al. (2014b) Neutrophil infiltration favors colitis-associated tumorigenesis by activating the interleukin-1 (IL-1)/IL-6 axis. Nature, 7(5): 1106-15.
Weinreich, D. M.; Elaraj, D. M.; Puhlmann, M.; Hewitt, S. M., et al. (2003) The role of interleukin 1 in growth and metastasis of human cancer xenografts. Cancer Res, 63(18):5957-5961.
Whitcomb, David C. (2004) Inflammation and Cancer. V. Chronic pancreatitis and pancreatic cancer. Am J Physiol Gastrointest Liver Physiol, 287:G315-G319.
(Wolf et al, 2001) Wolf J S, Chen Z, Dong G et al. (2001) IL (interleukin)-1alpha promotes nuclear factor-kappaB and AP-1-induced IL-8 expression, cell survival, and proliferation in head and neck squamous cell carcinomas. Clin Cancer Res, 7:1812-20.
Xu J1, Yin Z, Cao S, Gao W, et al. (2013) Systematic review and meta-analysis on the association between IL-1B polymorphisms and cancer risk. PLoS One. 2013 May 21; 8(5):e63654. doi: 10.1371/journal.pone.0063654.
Xu H, Ding Q, Jiang H W. (2014) Genetic polymorphism of interleukin-1A (IL-1A), IL-1B, and IL-1 receptor antagonist (IL-1RN) and prostate cancer risk. Asian Pac J Cancer Prev, 15(20):8741-7.
Lee, C. H., S. H. Syu, K. J. Liu, P. Y. Chu, W. C. Yang, P. Lin and W. Y. Shieh (2015). “Interleukin-1 beta transactivates epidermal growth factor receptor via the CXCL1-CXCR2 axis in oral cancer.” Oncotarget 6(36): 38866-38880.

### EXAMPLES

### Example 1: Incidence of Lung Cancer in IL-1 Positive and IL-1 Negative Populations of Smokers and Non-Smokers

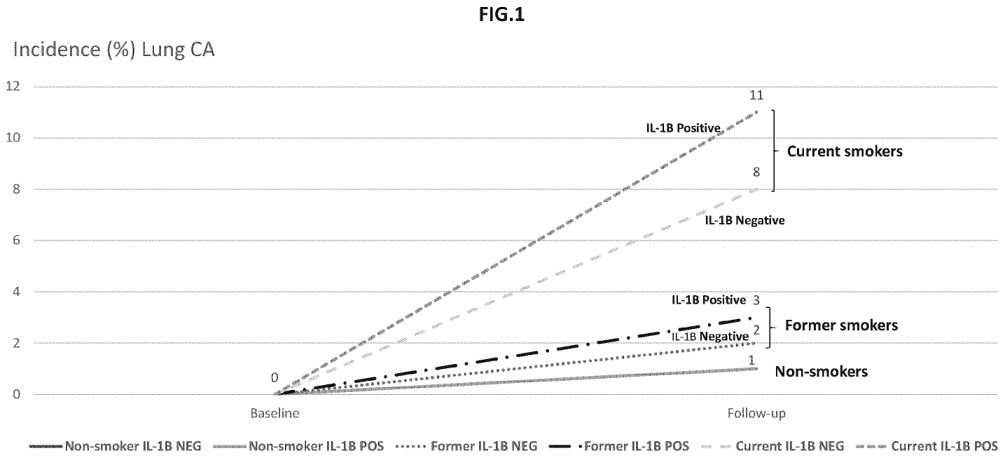

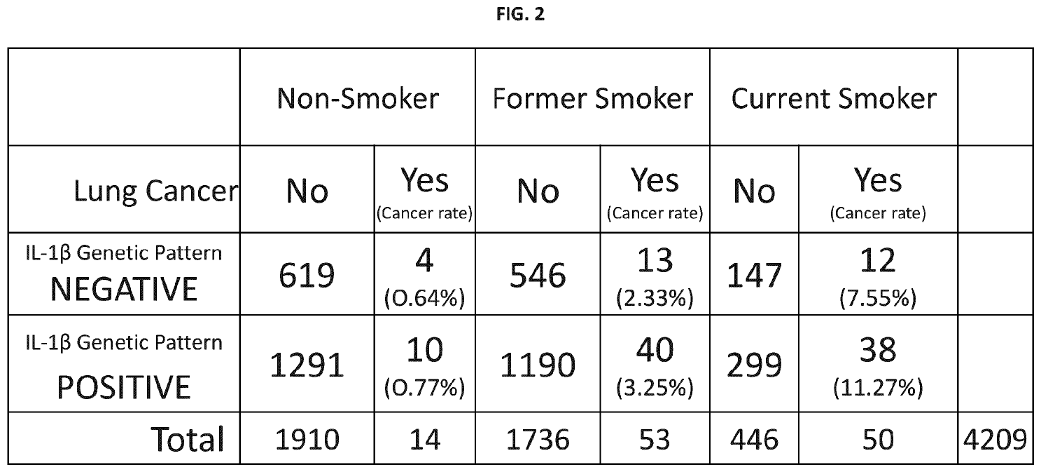

4,232 subjects were followed for a median of 14.7 years and assessed for lung cancer and smoking status. Participants were Caucasian men and women, and were drawn from the Atherosclerosis Risk in Communities (ARIC) study cohort (see Michaud et al. (2018) JNCI Natl Cancer Inst 11(8):djx278). IL-1 genotype was assessed for 4,209 study subjects using the rs4848306, rs1143623, and 16944 loci and the IL-1 genotypes shown in Table 3. Results are shown in FIG. 1 and FIG. 2.

### Other Embodiments

While the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of reducing a risk of developing lung cancer in a subject comprising:
(a) obtaining information regarding the subject's single nucleotide polymorphism (SNP) alleles for:
i. each of the rs17561 polymorphic locus, the rs16944 polymorphic locus and the rs1143634 polymorphic locus;
ii. each of the rs16944 polymorphic locus, the rs1143623 polymorphic locus and the rs4848306 polymorphic locus; or
iii. each of the rs17561 polymorphic locus, the rs16944 polymorphic locus the rs1143634 polymorphic locus, the rs1143623 polymorphic locus and the rs4848306 polymorphic locus;
(b) diagnosing the subject as at risk of developing lung cancer if the subject has a positive IL-1 genotype pattern obtained in (b) that is the same as any of:
i. T/T or T/G at rs17561, C/C, T/T, C/T or T/C at rs4848306, G/G at rs1143623, C/C at rs16944 and T/T or T/C at rs1143634;
ii. G/G at rs17561, C/C, T/T, C/T or T/C at rs4848306, G/G at rs1143623, C/C at rs16944 and C/C, T/T, C/T or T/C at rs1143634;
iii. G/G, T/T, G/T or T/G at rs17561, C/C, T/T, C/T or T/C at rs4848306, G/G at rs1143623, C/C at rs16944 and C/C at rs1143634;
iv. T/T or T/G at rs17561, C/C or C/T at rs4848306, G/G at rs1143623, C/T at rs16944 and T/T or T/C at rs1143634;
v. G/G at rs17561, C/C or C/T at rs4848306, G/G at rs1143623, C/T at rs16944 and C/C, T/T, C/T or T/C at rs1143634;
vi. G/G, T/T, G/T or T/G at rs17561, C/C or C/T at rs4848306, G/G at rs1143623, C/T at rs16944 and C/C at rs1143634;
vii. T/T or T/G at rs17561, C/C at rs4848306, C/G at rs1143623, C/T at rs16944 and T/T or T/C at rs1143634;
viii. G/G at rs17561, C/C at rs4848306, C/G at rs1143623, C/T at rs16944 and C/C, T/T, C/T or T/C at rs1143634;
ix. G/G, T/T, G/T or T/G at rs17561, C/C at rs4848306, C/G at rs1143623, C/T at rs16944 and C/C at rs1143634;
x. T/T or T/G at rs17561, C/C at rs4848306, G/G at rs1143623, T/T at rs16944 and T/T or T/C at rs1143634;
xi. G/G at rs17561, C/C at rs4848306, G/G at rs1143623, T/T at rs16944 and C/C, T/T, C/T or T/C at rs1143634;
xii. G/G, T/T, G/T or T/G at rs17561, C/C at rs4848306, G/G at rs1143623, T/T at rs16944 and C/C at rs1143634;
xiii. T/T or T/G at rs17561, C/C at rs16944 and T/T/ or T/C at rs1143634;
xiv. G/G at rs17561, C/C at rs16944 and C/C, T/T, C/T or T/C at rs1143634;
xv. G/G, T/T, G/T or T/G at rs17561, C/C at rs16944 and C/C at rs1143634
xvi. T/T or T/G at rs17561, C/T at rs16944 and T/T or T/C at rs1143634;
xvii. C/C, T/T, C/T or T/C at rs4848306, G/G at rs1143623, C/C at rs16944;
xviii. C/C or C/T at rs4848306, G/G at rs1143623, C/T at rs16944;
xix. C/C at rs4848306, C/G at rs1143623, C/T at rs16944; and
xx. C/C at rs4848306, G/G at rs1143623, T/T at rs16944; and
(c) administering a non-genetic lung cancer test to the subject diagnosed as having an IL-1 positive genotype pattern in step (b).

2. The method of claim 1, wherein the subject does not have a risk factor for lung cancer.

3. The method of claim 1, comprising identifying a subject who has a risk factor for lung cancer prior to step (a).

4. The method of claim 3, wherein the risk factor comprises an environmental risk factor, a genetic risk factor, a biomarker, a previous history of lung cancer, or lung nodules or masses.

5. The method of claim 4, wherein the environmental risk factor comprises a smoking history, exposure to second hand smoke, asbestos, radon or diesel exhaust, inhalation of carcinogenic chemicals or radioactive materials or previous radiation therapy directed to the thorax.

6. The method of claim 4, wherein the biomarker comprises an angiogenic factor, a lung cancer associated protein, an RNA, a DNA, a micro-RNA, an exosome, a circulating tumor cell or a change in metabolites.

7. The method of claim 4, wherein the genetic risk factor comprises a family history of lung cancer.

8. The method of claim 5, wherein the smoking history comprises less than 30 pack years.

9. The method of claim 5, wherein the smoking history comprises more than 15 years since quitting smoking.

10. The method of claim 8 or 9, wherein the subject is less than 55 or greater than 80 years of age.

11. The method of claim 5, wherein the smoking history comprises at least 30 pack years.

12. The method of claim 5, wherein the smoking history comprises less than 15 years since quitting.

13. The method of any one of claims 10-12, wherein the non-genetic lung cancer test comprises an imaging test or a test for a lung cancer biomarker.

14. The method of claim 11, wherein the imaging test comprises a chest X-ray, sputum cytology, magnetic resonance imaging (MRI) or fluorodeoxyglucose positron emission tomography computed tomography (PET/CT).

15. The method of claim 6, wherein the chest X-ray comprises a low-dose computed tomography (CT) scan for lung cancer or suspicious nodules, or a low-dose helical CT scan.

16. The method of claim 13, wherein the test for a lung cancer biomarker comprises testing for an angiogenic factor, one or more proteins associated with lung cancer, RNA, DNA, micro-RNA, exosome, circulating tumor cells or a change in metabolites associated with lung cancer.

17. The method of claim 16, wherein the one or more protein associated with lung cancer comprises advanced glycosylation end-product specific receptor (AGER), adipogenesis regulatory factor (C10orf116), adducin 2 (ADD2), periaxin (PRX), laminin subunit beta 3 (LAMB3), synemin (SYNM), spectrin alpha, erythrocytic 1 (SPTA1), ankyrin 1 (ANK1), hemoglobin subunit epsilon 1 (HBE1), hemoglobin subunit gamma 1 (HBG1), carbonic anhydrase 1 (CA1), tenascin XB (TNXB), multimerin 2 (MMRN2), hemoglobin subunit alpha 1 (HBA1), caveolin 1 (CAV1), hemoglobin subunit beta (HBB), collagen type VI alpha 6 chain (COL6A6), chromosome 1 open reading frame 198 (C1orf198), chloride intracellular channel 2 (CLIC2), caveolae associated protein 2 (SDPR), EH domain containing 2 (EHD2), apolipoprotein A2 (APOA2), NADH:ubiquinone oxidoreductase subunit B7 (NDUFB7), caveolae associated protein 3 (PRKCDBP), aminin subunit alpha 3 (LAMA3), EvC ciliary complex subunit 2 (LBN), four and a half LIM domains 5 (ACT), insulin like growth factor binding protein 3 (IGFBP3), prostaglandin D2 synthase (L-PGDS), serum amyloid A1 (SAA), retinoic acid receptor beta (HAP), hepatocyte growth factor (HGF), transthyretin (TTR), clusterin (CLU), tripartite motif containing 21 (SSA), apolipoprotein A4 (APOA4), ceruloplasmin (CP), haptoglobin (HP), keratin 2 (KRT2A), glutamate transporter 1b (GLT1B), casein kinase 1 alpha 1 (CK1), AKT serine/threonine kinase 1 (AKT), mannose binding lectin 2 (MBL2), tRNA-Leu (AAG) 1-2 (AAG1-2), fibrinogen alpha chain (FGA), gelsolin (GSN), ficolin 3 (FCN3), carnosine dipeptidase 1 (CNDP1), calcitonin related polypeptide alpha (CALCA), carbamoyl-phosphate synthase 1 (CPS1), chromogranin B (CHGB), involucrin (IVL), anterior gradient 2, protein disulphide isomerase family member (AGR2), nuclear autoantigenic sperm protein (NASP), phosphofructokinase, platelet (PFKP), thrombospondin 2 (THBS2), thioredoxin domain containing 17 (TXNDC17), proprotein convertase subtilisin/kexin type 1 (PCSK1), cellular retinoic acid binding protein 2 (CRABP2), acyl-CoA binding domain containing 3 (ACBD3), desmoglein 2 (DSG2), LPS responsive beige-like anchor protein (LRBA), serine/threonine kinase receptor associated protein (STRAP), VGF nerve growth factor (VGF), NOP2 nucleolar protein (NOP2), lipocalin 2 (LCN2), creatine kinase, mitochondrial 1B (CKMT1B), aldo-keto reductase family 1 member B10 (AKR1B10), proliferating cell nuclear antigen (PCNA), carboxypeptidase D (CPD), proteasome activator subunit 3 (PSME3), villin 1 (VIL1), serpin family B member 5 (SERPINB5), ribosomal protein L5 (RPL5), plakophilin 1 (PKP1), Ribosomal protein L10 (RPL10), aldo-keto reductase family 1 member C1 (AKR1C1), ribosomal protein S2 (RPS2), aldo-keto reductase family 1 member C3 (AKR1C3), visinin like 1 (VSNL1), adenosylhomocysteinase (AHCY), IMMP10, p21 (RAC1) activated kinase 2 (PAK2), isoleucyl-tRNA synthetase (IARS), proteasome 26S subunit, non-ATPase 2 (PSMD2), guanylate binding protein 5 (GBP5), minichromosome maintenance complex component 6 (MCM6), N-myc downstream regulated 1 (NDRG1), NOP58 ribonucleoprotein (NOP58), S100 calcium binding protein A2 (S100A2), Neuregulin 1 (NRG1), Neuregulin 2 (NRG2), ISG15 ubiquitin like modifier (UCRP), CER, plasminogen activator, urokinase (UPA), matrix metallopeptidase 14 (MT1-MMP), stratifin (SFN), transferrin (TF), albumin (ALB), S100 calcium binding protein A9 (S100A9), stathmin 1 (STMN), ENO, insulin like growth factor binding protein 7 (IGFBP7), or thrombospondin 1 (THBS1).

18. The method of any one of claims 1-17, wherein the testing is administered once a month, every 2 months, every 3 months, every 4 months, every 5 months, every 6 months, every 8 months, every 12 months, every 18 months, every 2 years, every 2.5 years or every 3 years.

19. The method of any one of claims 1-18, further comprising administering an inflammation inhibitor to the subject.

20. The method of claim 19, wherein the inflammation inhibitor is an IL-1, IL-6 inhibitor, a GM-CS inhibitor, or a JAK/STAT inhibitor.

21. The method of claim 19, wherein the inflammation inhibitor is formulated as an aerosol.

22. The method of claim 21, wherein the aerosol is administered as a nasal spray.

23. The method of any one of claims 20-22, wherein the IL-1 inhibitor is an IL-1β inhibitor or an IL-1α inhibitor.

24. The method of claim 23, wherein the IL-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, Canakinumab, a DOM4-130-201 and DOM4-130-202 antibody.

25. The method of claim 23, wherein the IL-1β inhibitor is Canakinumab or a derivative thereof.

26. The method of claim 23, wherein the IL-1α inhibitor is selected from the group consisting of Bermekimab, ABT-981, Isunakinra, AC-701, Sairei-To, Can-04, XL-130, a MABp1 antibody and Givinostat.

27. The method of claim 20, wherein the IL-6 inhibitor is selected from the group consisting of Tocilizumab, Siltuximab, Olokizumab, Elsilimomab, Sirukimab, Levilimab, ALX-0061, Gerilimzumab and Sarilumab.

28. A method of reducing a risk of developing colorectal cancer in a subject comprising:
(a) obtaining information regarding the subject's single nucleotide polymorphism (SNP) alleles for:
i. each of the rs17561 polymorphic locus, the rs16944 polymorphic locus and the rs1143634 polymorphic locus;
ii. each of the rs16944 polymorphic locus, the rs1143623 polymorphic locus and the rs4848306 polymorphic locus; or
iii. each of the rs17561 polymorphic locus, the rs16944 polymorphic locus the rs1143634 polymorphic locus, the rs1143623 polymorphic locus and the rs4848306 polymorphic locus;
(b) diagnosing the subject as at risk of developing colorectal cancer if the subject has a positive IL-1 genotype pattern obtained in (b) that is the same as any of:
i. T/T or T/G at rs17561, C/C, T/T, C/T or T/C at rs4848306, G/G at rs1143623, C/C at rs16944 and T/T or T/C at rs1143634;
ii. G/G at rs17561, C/C, T/T, C/T or T/C at rs4848306, G/G at rs1143623, C/C at rs16944 and C/C, T/T, C/T or T/C at rs1143634;
iii. G/G, T/T, G/T or T/G at rs17561, C/C, T/T, C/T or T/C at rs4848306, G/G at rs1143623, C/C at rs16944 and C/C at rs1143634;
iv. T/T or T/G at rs17561, C/C or C/T at rs4848306, G/G at rs1143623, C/T at rs16944 and T/T or T/C at rs1143634;
v. G/G at rs17561, C/C or C/T at rs4848306, G/G at rs1143623, C/T at rs16944 and C/C, T/T, C/T or T/C at rs1143634;
vi. G/G, T/T, G/T or T/G at rs17561, C/C or C/T at rs4848306, G/G at rs1143623, C/T at rs16944 and C/C at rs1143634;
vii. T/T or T/G at rs17561, C/C at rs4848306, C/G at rs1143623, C/T at rs16944 and T/T or T/C at rs1143634;
viii. G/G at rs17561, C/C at rs4848306, C/G at rs1143623, C/T at rs16944 and C/C, T/T, C/T or T/C at rs1143634;
ix. G/G, T/T, G/T or T/G at rs17561, C/C at rs4848306, C/G at rs1143623, C/T at rs16944 and C/C at rs1143634;
x. T/T or T/G at rs17561, C/C at rs4848306, G/G at rs1143623, T/T at rs16944 and T/T or T/C at rs1143634;
xi. G/G at rs17561, C/C at rs4848306, G/G at rs1143623, T/T at rs16944 and C/C, T/T, C/T or T/C at rs1143634;
xii. G/G, T/T, G/T or T/G at rs17561, C/C at rs4848306, G/G at rs1143623, T/T at rs16944 and C/C at rs1143634;
xiii. T/T or T/G at rs17561, C/C at rs16944 and T/T/ or T/C at rs1143634;
xiv. G/G at rs17561, C/C at rs16944 and C/C, T/T, C/T or T/C at rs1143634;
xv. G/G, T/T, G/T or T/G at rs17561, C/C at rs16944 and C/C at rs1143634
xvi. T/T or T/G at rs17561, C/T at rs16944 and T/T or T/C at rs1143634;
xvii. C/C, T/T, C/T or T/C at rs4848306, G/G at rs1143623, C/C at rs16944;
xviii. C/C or C/T at rs4848306, G/G at rs1143623, C/T at rs16944;
xix. C/C at rs4848306, C/G at rs1143623, C/T at rs16944; and
xx. C/C at rs4848306, G/G at rs1143623, T/T at rs16944; and
(c) administering a non-genetic colorectal cancer test to the subject diagnosed as having an IL-1 positive genotype pattern in step (b).

29. The method of claim 28, wherein the subject does not have a risk factor for colorectal cancer.

30. The method of claim 28, wherein the subject has one or more risk factors for colorectal cancer.

31. The method of claim 30, wherein the one or more risk factors comprise being overweight or obese, lack of physical activity, diet, smoking, heavy alcohol use, age over fifty, a history of adenomatous polyps, a family history of adenomatous polyps, a previous diagnosis of colorectal cancer, a family history of colorectal cancer, a history of inflammatory bowel disease, type II diabetes, radiation therapy to treat prostate cancer or a genetic predisposition to colorectal cancer.

32. The method of claim 31, wherein the genetic predisposition to colorectal cancer comprises Lynch syndrome, familial adenomatous polyposis (FAP), or a mutation in serine/threonine kinase 11 (LBK1), mutY DNA glycosylase (MUTYH) or SMAD family member 4 (SMAD4).

33. The method of claim 32, wherein the Lynch syndrome comprises a mutL homolog 1 (MLH1) mutation or a mutS homolog 2 (MSH2) mutation.

34. The method of claim 32, wherein the FAP comprises a mutation in the adenomatous polyposis coli (APC) gene.

35. The method of claim 31, wherein the diet comprises a diet high in red meat or processed meat, or both.

36. The method of any one of claims 28-35, wherein the non-genetic colorectal cancer test comprises a high-sensitivity fecal occult blood test (FOBT), a fecal immunochemical test (FIT), a sigmoidoscopy, a colonoscopy, computed tomographic (CT) colonography, a double contrast barium enema or a blood test.

37. The method of claim 36, wherein the FIT tests for at least one DNA marker associated with colorectal cancer.

38. The method of claim 37, wherein the at least one DNA marker associated with colorectal cancer comprises an alteration in APC, catenin beta 1 (CTNNB1), KRAS proto-oncogene, GTPase (KRAS), B-Raf proto-oncogene, serine/threonine kinase (BRAF), SMAD4, transforming growth factor beta receptor 2 (TGFBR2), tumor protein p53 (TP53), phosphatidylinositol-4,5-bisphosphate 3-kinase catalytic subunit alpha (PIK3CA), AT-rich interaction domain 1A (ARID1A), SRY-box 9 (SOX9), APC membrane recruitment protein 1 (FAM123B), erb-b2 receptor tyrosine kinase 2 (ERBB2), vimentin (VIM), NDRG family member 4 (NDRG4), septin 9 (SEPT9), bone morphogenetic protein 3 (BMP3) or tissue factor pathway inhibitor 2 (TFPI2).

39. The method of claim 38, wherein the alteration comprises a mutation or a change in DNA methylation.

40. The method of claim 36, wherein the FIT or FOBT comprises an immunoassay for hemoglobin.

41. The method of claim 36, wherein the blood test comprises testing for a biomarker associated with colorectal cancer.

42. The method of claim 41, wherein the biomarker comprises methylated SEPT9 DNA.

43. The method of any one of claims 28-42, wherein the testing is administered once a month, every 2 months, every 3 months, every 4 months, every 5 months, every 6 months, every 8 months, every 12 months, every 18 months, every 2 years, every 2.5 years or every 3 years.

44. The method of any one of claims 28-43, wherein the method further comprises administering an inflammation inhibitor.

45. The method of claim 44, wherein the inflammation inhibitor is an IL-1 inhibitor, an IL-6 inhibitor, a GM-CSF inhibitor, or a JAK/STAT inhibitor.

46. The method of claim 45, wherein the IL-1 inhibitor is an IL-1α inhibitor or an IL-1β inhibitor.

47. The method of claim 46, wherein the IL-1α inhibitor is selected from the group consisting of Bermekimab, ABT-981, Isunakinra, AC-701, Sairei-To, Can-04, XL-130, a MABp1 antibody and Givinostat.

48. The method of claim 46, wherein the IL-1α inhibitor is Bermekimab.

49. The method of claim 46, wherein the IL-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, Canakinumab, a DOM4-130-201 and DOM4-130-202 antibody.

50. The method of claim 45, wherein the IL-6 inhibitor is selected from the group consisting of Tocilizumab, Siltuximab, Olokizumab, Elsilimomab, Sirukimab, Levilimab, ALX-0061, Gerilimzumab and Sarilumab.

51. A method of treating lung cancer in a subject comprising:
(a) identifying a subject who has lung cancer;
(b) obtaining information regarding the subject's single nucleotide polymorphism (SNP) alleles for:
i. each of the rs17561 polymorphic locus, the rs16944 polymorphic locus and the rs1143634 polymorphic locus;
ii. each of the rs16944 polymorphic locus, the rs1143623 polymorphic locus and the rs4848306 polymorphic locus; or
iii. each of the rs17561 polymorphic locus, the rs16944 polymorphic locus the rs1143634 polymorphic locus, the rs1143623 polymorphic locus and the rs4848306 polymorphic locus;
(c) diagnosing the subject as having a positive IL-1 genotype pattern if the SNP alleles obtained in (b) are the same as any of:
i. T/T or T/G at rs17561, C/C, T/T, C/T or T/C at rs4848306, G/G at rs1143623, C/C at rs16944 and T/T or T/C at rs1143634;
ii. G/G at rs17561, C/C, T/T, C/T or T/C at rs4848306, G/G at rs1143623, C/C at rs16944 and C/C, T/T, C/T or T/C at rs1143634;
iii. G/G, T/T, G/T or T/G at rs17561, C/C, T/T, C/T or T/C at rs4848306, G/G at rs1143623, C/C at rs16944 and C/C at rs1143634;
iv. T/T or T/G at rs17561, C/C or C/T at rs4848306, G/G at rs1143623, C/T at rs16944 and T/T or T/C at rs1143634;
v. G/G at rs17561, C/C or C/T at rs4848306, G/G at rs1143623, C/T at rs16944 and C/C, T/T, C/T or T/C at rs1143634;
vi. G/G, T/T, G/T or T/G at rs17561, C/C or C/T at rs4848306, G/G at rs1143623, C/T at rs16944 and C/C at rs1143634;
vii. T/T or T/G at rs17561, C/C at rs4848306, C/G at rs1143623, C/T at rs16944 and T/T or T/C at rs1143634;
viii. G/G at rs17561, C/C at rs4848306, C/G at rs1143623, C/T at rs16944 and C/C, T/T, C/T or T/C at rs1143634;
ix. G/G, T/T, G/T or T/G at rs17561, C/C at rs4848306, C/G at rs1143623, C/T at rs16944 and C/C at rs1143634;
x. T/T or T/G at rs17561, C/C at rs4848306, G/G at rs1143623, T/T at rs16944 and T/T or T/C at rs1143634;
xi. G/G at rs17561, C/C at rs4848306, G/G at rs1143623, T/T at rs16944 and C/C, T/T, C/T or T/C at rs1143634;
xii. G/G, T/T, G/T or T/G at rs17561, C/C at rs4848306, G/G at rs1143623, T/T at rs16944 and C/C at rs1143634;
xiii. T/T or T/G at rs17561, C/C at rs16944 and T/T/ or T/C at rs1143634;
xiv. G/G at rs17561, C/C at rs16944 and C/C, T/T, C/T or T/C at rs1143634;
xv. G/G, T/T, G/T or T/G at rs17561, C/C at rs16944 and C/C at rs1143634
xvi. T/T or T/G at rs17561, C/T at rs16944 and T/T or T/C at rs1143634;
xvii. C/C, T/T, C/T or T/C at rs4848306, G/G at rs1143623, C/C at rs16944;
xviii. C/C or C/T at rs4848306, G/G at rs1143623, C/T at rs16944;
xix. C/C at rs4848306, C/G at rs1143623, C/T at rs16944; and
xx. C/C at rs4848306, G/G at rs1143623, T/T at rs16944; and
(d) administering an inflammation inhibitor to the subject diagnosed as having a positive IL-1 genotype pattern in (c).

52. The method of claim 51, wherein identifying a subject who has lung cancer comprises:
(i) identifying a subject who has one or more risk factors or biomarkers of lung cancer; and
(ii) testing the subject for lung cancer.

53. The method of claim 52, wherein the risk factor comprises an environmental risk factor, a genetic risk factor, a biomarker, a previous history of lung cancer, or lung nodules or masses.

54. The method of claim 53, wherein the environmental risk factor comprises a smoking history, exposure to second hand smoke, asbestos, radon or diesel exhaust, inhalation of carcinogenic chemicals or radioactive materials or previous radiation therapy directed to the thorax.

55. The method of claim 53, wherein the genetic risk factor comprises a family history of lung cancer.

56. The method of claim 52, wherein the biomarker comprises an angiogenic factor, one or more lung cancer associated proteins, an RNA, a DNA, a micro-RNA, an exosome, a circulating tumor cell or a change in metabolites.

57. The method of claim 56, wherein the one or more proteins comprise AGER, C10orf116, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, UPA, MT1-MMP, SFN, TF, ALB, S100A9, STMN, ENO, IGFBP7, or THBS1.

58. The method of claim 52, wherein the risk factor comprises a lung nodule, lung tumor, lung mass, evidence of angiogenesis or evidence of tumor invasion of other tissues.

59. The method of any one of claims 52-58, wherein testing the subject for lung cancer comprises a biopsy of a lung tumor.

60. The method of any one of claims 51-59, wherein the inflammation inhibitor is an IL-1 inhibitor, an IL-6 inhibitor, a GM-CSF inhibitor, or a JAK/STAT inhibitor.

61. The method of any one of claims 51-60, wherein the inflammation inhibitor is formulated as an aerosol.

62. The method of claim 61, wherein the aerosol is administered as a nasal spray.

63. The method of any one of claims 60-62, wherein the IL-1 inhibitor is an IL-1β inhibitor or an IL-1α inhibitor.

64. The method of claim 63, wherein the IL-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201, a DOM4-130-202 antibody and Canakinumab.

65. The method of claim 63, wherein the IL-1β inhibitor is Canakinumab or a derivative thereof.

66. The method of claim 65, wherein the Canakinumab is administered to the subject at a dose of 25 mg to 300 mg.

67. The method of claim 65, wherein the subject weighs less than 40 kg and the Canakinumab is administered to the subject at a dose of 2 mg/kg or 4 mg/kg.

68. The method of claim 65, wherein the subject weighs more than 40 kg and the Canakinumab is administered to the subject at a dose of 150 mg or 300 mg.

69. The method of any one of claims 65-68, wherein the Canakinumab is administered every 2 weeks, every 4 weeks, every 6 weeks, every 8 weeks, every 10 weeks, every 3 months, every 5 months or every 6 months from the first administration.

70. The method of any one of claims 65-68, wherein the Canakinumab is administered every 4 weeks from the first administration.

71. The method of any one of claims 65-68, wherein the Canakinumab is administered parenterally.

72. The method of claim 71, wherein the Canakinumab is administered by intravenous injection, intravenous infusion, intramuscularly, via intrapulmonary administration or subcutaneously.

73. The method of claim 63, wherein the IL-1α inhibitor is selected from the group consisting of Bermekimab, ABT-981, Isunakinra, AC-701, Sairei-To, Can-04, XL-130, a MABp1 antibody and Givinostat.

74. The method of claim 60, wherein the IL-6 inhibitor is selected from the group consisting of Tocilizumab, Siltuximab, Olokizumab, Elsilimomab, Sirukimab, Levilimab, ALX-0061, Gerilimzumab and Sarilumab.

75. The method of any one of claims 51-75, wherein the lung cancer is stage 0, stage 1, stage 2, stage 3 or stage 4 lung cancer.

76. The method of any one of claims 51-75, wherein administering the inflammation inhibitor reduces a sign or a symptom of the cancer.

77. The method of any one of claims 51-76, wherein administering the inflammation inhibitor reduces a number of tumors of the cancer, reduces a size of a tumor of the cancer, reduces a growth rate of a tumor of the cancer, reduces early metaplastic changes in the cancer, reduces neo-angiogenesis, reduces tissue invasiveness by the cancer, reduces tissue invasion by the cancer through a basement membrane, reduces invasion of bone by the cancer, reduces metastasis of the cancer to distant organs, or a combination thereof.

78. The method of any one of claims 51-77, wherein administering the inflammation inhibitor reduces a level of one or more biomarkers associated with lung cancer.

79. The method of claim 78, wherein the biomarker comprises AGER, C10orf116, ADD2, PRX, LAMB3, SYNM, SPTA1, ANK1, HBE1, HBG1, CA1, TNXB, MMRN2, HBA1, CAV1, HBB, COL6A6, C1orf198, CLIC2, SDPR, EHD2, APOA2, NDUFB7, PRKCDBP, LAMA3, LBN, ACT, IGFBP3, L-PGDS, SAA, HAP, HGF, TTR, CLU, SSA, APOA4, CP, HP, KRT2A, GLT1B, CK1, AKT, MBL2, AAG1-2, FGA, GSN, FCN3, CNDP1, CALCA, CPS1, CHGB, IVL, AGR2, NASP, PFKP, THBS2, TXNDC17, PCSK1, CRABP2, ACBD3, DSG2, LRBA, STRAP, VGF, NOP2, LCN2, CKMT1B, AKR1B10, PCNA, CPD, PSME3, VIL1, SERPINB5, RPL5, PKP1, RPL10, AKR1C1, RPS2, AKR1C3, VSNL1, AHCY, IMMP10, PAK2, IARS, PSMD2, GBP5, MCM6, NDRG1, NOP58, S100A2, NRG1, NRG2, UCRP, CER, UPA, MT1-MMP, SFN, TF, ALB, S100A9, STMN, ENO, IGFBP7, or THBS1.

80. A method of treating colorectal cancer in a subject comprising:
(a) identifying a subject who has colorectal cancer or a risk for colorectal cancer;
(b) obtaining information regarding the subject's single nucleotide polymorphism (SNP) alleles for:
i. each of the rs17561 polymorphic locus, the rs16944 polymorphic locus and the rs1143634 polymorphic locus;
ii. each of the rs16944 polymorphic locus, the rs1143623 polymorphic locus and the rs4848306 polymorphic locus; or
iii. each of the rs17561 polymorphic locus, the rs16944 polymorphic locus the rs1143634 polymorphic locus, the rs1143623 polymorphic locus and the rs4848306 polymorphic locus;
(c) diagnosing the subject as having a positive IL-1 genotype pattern if the SNP alleles obtained in (b) are the same as any of:
i. T/T or T/G at rs17561, C/C, T/T, C/T or T/C at rs4848306, G/G at rs1143623, C/C at rs16944 and T/T or T/C at rs1143634;
ii. G/G at rs17561, C/C, T/T, C/T or T/C at rs4848306, G/G at rs1143623, C/C at rs16944 and C/C, T/T, C/T or T/C at rs1143634;
iii. G/G, T/T, G/T or T/G at rs17561, C/C, T/T, C/T or T/C at rs4848306, G/G at rs1143623, C/C at rs16944 and C/C at rs1143634;
iv. T/T or T/G at rs17561, C/C or C/T at rs4848306, G/G at rs1143623, C/T at rs16944 and T/T or T/C at rs1143634;
v. G/G at rs17561, C/C or C/T at rs4848306, G/G at rs1143623, C/T at rs16944 and C/C, T/T, C/T or T/C at rs1143634;
vi. G/G, T/T, G/T or T/G at rs17561, C/C or C/T at rs4848306, G/G at rs1143623, C/T at rs16944 and C/C at rs1143634;
vii. T/T or T/G at rs17561, C/C at rs4848306, C/G at rs1143623, C/T at rs16944 and T/T or T/C at rs1143634;
viii. G/G at rs17561, C/C at rs4848306, C/G at rs1143623, C/T at rs16944 and C/C, T/T, C/T or T/C at rs1143634;
ix. G/G, T/T, G/T or T/G at rs17561, C/C at rs4848306, C/G at rs1143623, C/T at rs16944 and C/C at rs1143634;
x. T/T or T/G at rs17561, C/C at rs4848306, G/G at rs1143623, T/T at rs16944 and T/T or T/C at rs1143634;
xi. G/G at rs17561, C/C at rs4848306, G/G at rs1143623, T/T at rs16944 and C/C, T/T, C/T or T/C at rs1143634;
xii. G/G, T/T, G/T or T/G at rs17561, C/C at rs4848306, G/G at rs1143623, T/T at rs16944 and C/C at rs1143634;
xiii. T/T or T/G at rs17561, C/C at rs16944 and T/T/ or T/C at rs1143634;
xiv. G/G at rs17561, C/C at rs16944 and C/C, T/T, C/T or T/C at rs1143634;
xv. G/G, T/T, G/T or T/G at rs17561, C/C at rs16944 and C/C at rs1143634
xvi. T/T or T/G at rs17561, C/T at rs16944 and T/T or T/C at rs1143634;
xvii. C/C, T/T, C/T or T/C at rs4848306, G/G at rs1143623, C/C at rs16944;
xviii. C/C or C/T at rs4848306, G/G at rs1143623, C/T at rs16944;
xix. C/C at rs4848306, C/G at rs1143623, C/T at rs16944; and
xx. C/C at rs4848306, G/G at rs1143623, T/T at rs16944; and
(d) administering an inflammation inhibitor to the subject to the subject diagnosed as having an IL-1 positive genotype pattern in (c) and a positive screening assessment for colorectal cancer.

81. The method of claim 80, wherein identifying a subject who has colorectal cancer comprises:
(i) identifying a subject who has one or more risk factors or biomarkers of colorectal cancer; and
(ii) testing the subject for colorectal cancer.

82. The method of claim 81, wherein the one or more risk factors comprise being overweight or obese, lack of physical activity, diet, smoking, heavy alcohol use, age over fifty, a personal history of adenomatous polyps, a family history of adenomatous polyps or hereditary non-polyposis colon cancer, a previous diagnosis of colorectal cancer, a family history of colorectal cancer, a history of inflammatory bowel disease, type II diabetes, radiation therapy to treat prostate cancer, a genetic predisposition to colorectal cancer or one testing positive for one or more indicators of colorectal cancer.

83. The method of claim 82, wherein the genetic predisposition to colorectal cancer comprises Lynch syndrome, familial adenomatous polyposis (FAP), or a mutation in LBK1, MUTYH or SMAD4.

84. The method of claim 83, wherein the Lynch syndrome comprises an MLH1 mutation or an MSH2 mutation.

85. The method of claim 83, wherein the FAP comprises a mutation in the adenomatous polyposis coli (APC) gene.

86. The method of claim 82, wherein the diet comprises a diet high in red meat, processed meat, meat cooked at high temperature, or a combination thereof.

87. The method of any one of claims 81-86, wherein the biomarker for colorectal cancer is assayed using a high-sensitivity fecal occult blood test (FOBT), a fecal immunochemical test (FIT), a sigmoidoscopy, a colonoscopy screening for colon cancer or suspicious polyps, computed tomographic (CT) colonography, a double contrast barium enema or a blood test.

88. The method of claim 87, wherein the FIT tests for at least one DNA marker associated with colorectal cancer.

89. The method of claim 88, wherein the at least one DNA marker associated with colorectal cancer comprises an a mutation or a change in methylation in APC, CTNNB1, KRAS, BRAF, SMAD4, TGFBR2, TP53, PIK3CA, ARID1A, SOX9, FAM123B, ERBB2, VIM, NDRG4, SEPT9, BMP3 or TFPI2.

90. The method of claim 88, wherein the biomarker comprises methylated SEPT9 DNA.

91. The method of claim 87, wherein the FIT or the FOBT comprises an immunoassay for hemoglobin.

92. The method of any one of claims 81-91, wherein testing the subject for colorectal cancer comprises a biopsy.

93. The method of any one of claims 80-92, wherein the inflammation inhibitor is an IL-1 inhibitor, an IL-6 inhibitor, a GM-CSF inhibitor, or a JAK/STAT inhibitor.

94. The method of any one of claims 80-93, wherein the inflammation inhibitor is formulated as an aerosol.

95. The method of claim 94, wherein the aerosol is administered as a nasal spray.

96. The method of any one of claims 93-95, wherein the IL-1 inhibitor is an IL-1β inhibitor or an IL-1α inhibitor.

97. The method of claim 96, wherein the IL-1α inhibitor is selected from the group consisting of Bermekimab, ABT-981, Isunakinra, AC-701, Sairei-To, Can-04, XL-130, a MABp1 antibody and Givinostat.

98. The method of claim 96, wherein the IL-1α inhibitor is Bermekimab.

99. The method of claim 98, wherein the Bermekimab is administered at between 3 mg/kg to 20 mg/kg.

100. The method of claim 98 or 99, wherein the Bermekimab is administered at 7.5 mg/kg.

101. The method of any one of claims 98-100, wherein the Bermekimab is administered parenterally.

102. The method of claim 101, wherein the parenteral administration comprises subcutaneous injection, intramuscular injection, intravenous injection or intravenous infusion.

103. The method of any one of claims 98-102, wherein the Bermekimab is administered every week, every two weeks, every three weeks, every 4 weeks, every 5 weeks, every 6 weeks or every 8 weeks.

104. The method of claim 96, wherein the L-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201, a DOM4-130-202 antibody and Canakinumab.

105. The method of claim 96, wherein the IL-1β inhibitor is Canakinumab or a derivative thereof.

106. The method of any one of claims 80-105, wherein the colorectal cancer is stage 0, stage 1, stage 2, stage 3 or stage 4 colorectal cancer.

107. The method of any one of claims 80-106, wherein administering the inflammation inhibitor reduces a sign or a symptom of the colorectal cancer.

108. The method of any one of claims 80-107, wherein administering the inflammation inhibitor reduces a number of tumors of the cancer, reduces a size of a tumor of the cancer, reduces a growth rate of a tumor of the cancer, reduces early metaplastic changes in the cancer, reduces neo-angiogenesis, reduces tissue invasiveness by the cancer, reduces tissue invasion by the cancer through a basement membrane, reduces invasion of bone by the cancer, reduces metastasis of the cancer to distant organs, or a combination thereof.

109. The method of any one of claims 80-108, wherein administering the inflammation inhibitor reduces one or more biomarkers associated with colorectal cancer.

110. The method of claim 109, wherein the biomarker comprises a mutation or change in methylation state of APC, CTNNB1, KRAS, BRAF, SMAD4, TGFBR2, TP53, PIK3CA, ARID1A, SOX9, FAM123B, ERBB2, VIM, NDRG4, SEPT9, BMP3 or TFPI2.

111. A method of reducing a risk of metastatic breast cancer in a subject comprising:
(a) identifying a subject who has breast cancer;
(b) obtaining information regarding the subject's single nucleotide polymorphism (SNP) alleles for:
i. each of the rs17561 polymorphic locus, the rs16944 polymorphic locus and the rs1143634 polymorphic locus;
ii. each of the rs16944 polymorphic locus, the rs1143623 polymorphic locus and the rs4848306 polymorphic locus; or
iii. each of the rs17561 polymorphic locus, the rs16944 polymorphic locus the rs1143634 polymorphic locus, the rs1143623 polymorphic locus and the rs4848306 polymorphic locus;
(c) diagnosing the subject as at risk for metastatic breast cancer if the subject has a positive IL-1 genotype pattern obtained in (b) that is the same as any of:
i. T/T or T/G at rs17561, C/C, T/T, C/T or T/C at rs4848306, G/G at rs1143623, C/C at rs16944 and T/T or T/C at rs1143634;
ii. G/G at rs17561, C/C, T/T, C/T or T/C at rs4848306, G/G at rs1143623, C/C at rs16944 and C/C, T/T, C/T or T/C at rs1143634;
iii. G/G, T/T, G/T or T/G at rs17561, C/C, T/T, C/T or T/C at rs4848306, G/G at rs1143623, C/C at rs16944 and C/C at rs1143634;
iv. T/T or T/G at rs17561, C/C or C/T at rs4848306, G/G at rs1143623, C/T at rs16944 and T/T or T/C at rs1143634;
v. G/G at rs17561, C/C or C/T at rs4848306, G/G at rs1143623, C/T at rs16944 and C/C, T/T, C/T or T/C at rs1143634;
vi. G/G, T/T, G/T or T/G at rs17561, C/C or C/T at rs4848306, G/G at rs1143623, C/T at rs16944 and C/C at rs1143634;
vii. T/T or T/G at rs17561, C/C at rs4848306, C/G at rs1143623, C/T at rs16944 and T/T or T/C at rs1143634;
viii. G/G at rs17561, C/C at rs4848306, C/G at rs1143623, C/T at rs16944 and C/C, T/T, C/T or T/C at rs1143634;
ix. G/G, T/T, G/T or T/G at rs17561, C/C at rs4848306, C/G at rs1143623, C/T at rs16944 and C/C at rs1143634;
x. T/T or T/G at rs17561, C/C at rs4848306, G/G at rs1143623, T/T at rs16944 and T/T or T/C at rs1143634;
xi. G/G at rs17561, C/C at rs4848306, G/G at rs1143623, T/T at rs16944 and C/C, T/T, C/T or T/C at rs1143634;
xii. G/G, T/T, G/T or T/G at rs17561, C/C at rs4848306, G/G at rs1143623, T/T at rs16944 and C/C at rs1143634;
xiii. T/T or T/G at rs17561, C/C at rs16944 and T/T/ or T/C at rs1143634;
xiv. G/G at rs17561, C/C at rs16944 and C/C, T/T, C/T or T/C at rs1143634;
xv. G/G, T/T, G/T or T/G at rs17561, C/C at rs16944 and C/C at rs1143634
xvi. T/T or T/G at rs17561, C/T at rs16944 and T/T or T/C at rs1143634;
xvii. C/C, T/T, C/T or T/C at rs4848306, G/G at rs1143623, C/C at rs16944;
xviii. C/C or C/T at rs4848306, G/G at rs1143623, C/T at rs16944;
xix. C/C at rs4848306, C/G at rs1143623, C/T at rs16944; and
xx. C/C at rs4848306, G/G at rs1143623, T/T at rs16944; and
(d) administering an inflammation inhibitor to the subject to the subject diagnosed with a positive IL-1 genotype pattern in step (c).

112. The method of claim 111, wherein the breast cancer comprises a carcinoma, a sarcoma, a Phyllodes tumor, Paget disease, an angiosarcoma or an inflammatory breast cancer.

113. The method of claim 111 or 112, wherein the breast cancer is a pre-metastatic stage 0, stage I, stage II or stage III breast cancer.

114. The method of any one of claims 111-113, wherein the inflammation inhibitor is an IL-1 inhibitor, an IL-6 inhibitor, a GM-CSF inhibitor, or a JAK/STAT inhibitor.

115. The method of claim 114, wherein the IL-1 inhibitor is an IL-1α inhibitor or an IL-13 inhibitor.

116. The method of claim 115, wherein the L-1β inhibitor is selected from the group consisting of ABT-981, Anakinra, Anakinra Biosimilar, APX-002, binimetinib, CAN-04, Diacerein, DLX-2681, Givinostat, Isunakinra, Rilonacept, SER-140, XL-130, Gevokizumab, Can-04, a DOM4-130-201 antibody, DOM4-130-202 antibody and Canakinumab.

117. The method of claim 115, wherein the IL-1β inhibitor is Canakinumab or a derivative thereof.

118. The method of claim 117, wherein the Canakinumab is administered to the subject at a dose of 25 mg to 300 mg.

119. The method of claim 117, wherein the subject weighs less than 40 kg and the Canakinumab is administered to the subject at a dose of 2 mg/kg or 4 mg/kg.

120. The method of claim 117, wherein the subject weighs more than 40 kg and the Canakinumab is administered to the subject at a dose of 150 mg or 300 mg.

121. The method of any one of claims 117-120, wherein the Canakinumab is administered every 2 weeks, every 4 weeks, every 6 weeks, every 8 weeks, every 10 weeks, every 3 months, every 5 months or every 6 months from the first administration.

122. The method of any one of claims 117-120, wherein the Canakinumab is administered every 4 weeks from the first administration.

123. The method of any one of claims 117-122, wherein the Canakinumab is administered parenterally.

124. The method of claim 123, wherein the Canakinumab is administered by intravenous injection, intravenous infusion, intramuscularly or subcutaneously.

125. The method of claim 115, wherein the IL-1α inhibitor is selected from the group consisting of Bermekimab, ABT-981, Isunakinra, AC-701, Sairei-To, Can-04, XL-130, a MABp1 antibody and Givinostat.

126. The method of claim 115, wherein the IL-1α inhibitor is Bermekimab.

127. The method of claim 126, wherein the Bermekimab is administered at between 3 mg/kg to 20 mg/kg.

128. The method of claim 126, wherein the Bermekimab is administered at 7.5 mg/kg.

129. The method of any one of claims 126-128, wherein the Bermekimab is administered parenterally.

130. The method of claim 129, wherein the parenteral administration comprises subcutaneous injection, intramuscular injection, intravenous injection or intravenous infusion.

131. The method of any one of claims 126-130, wherein the Bermekimab is administered every week, every two weeks, every three weeks, every 4 weeks, every 5 weeks, every 6 weeks or every 8 weeks.

132. The method of claim 114, wherein the IL-6 inhibitor is selected from the group consisting of Tocilizumab, Siltuximab, Olokizumab, Elsilimomab, Sirukimab, Levilimab, ALX-0061, Gerilimzumab and Sarilumab.

133. The method of any one of claims 111-132, wherein administering the inflammation inhibitor reduces a sign or a symptom of the breast cancer.

134. The method of any one of claims 111-132, wherein administering the inflammation inhibitor reduces metastasis of the breast cancer.

135. The method of any one of claims 51-134, further comprising chemotherapy, radiation treatment, surgical removal of the cancer, an immunotherapy, an immune checkpoint inhibitor, a therapeutic vaccine, an antibody therapy, or a combination thereof.

136. The method of claim 135, wherein the chemotherapy comprises a taxane, a platinum agent, an alkylating agent, a mitotic inhibitor, an antimetabolite, an alkaloid, an antitumor antibiotic, a topoisomerase inhibitor, a tyrosine kinase inhibitor, an mTOR inhibitor, a B-Raf inhibitor, an EGFR inhibitor, a PARP inhibitor, a phosphoinositide 3-kinase (PI3K) inhibitor, a CDK inhibitor or a combination thereof.

137. The method of claim 136, wherein the topoisomerase inhibitor comprises doxorubicin, epirubicin, irinotecan, topotecan, mitoxantrone, daunorubicin or etoposide.

138. The method of claim 136, wherein the alkylating agent comprises cyclophosphamide, chlorambucil, melphalan, ifosfamide or mechlorethamine hydrochloride.

139. The method of claim 136, wherein the antimetabolite comprises pemetrexed, gemcitabine, methotrexate, 5-fluorouracil, capecitabine or trifluridine and tipiracil.

140. The method of claim 136, wherein the alkaloid comprises actinomycin D, doxorubicin or mitomycin, vinorelbine or vinblastine.

141. The method of claim 136, wherein the antitumor antibiotic comprises doxorubicin, mitoxantrone or bleomycin.

142. The method of claim 136, wherein the taxane comprises taxol, docetaxel or paclitaxel.

143. The method of claim 136, wherein the tyrosine kinase inhibitor comprises afatinib, apatinib, alectinib, brigantinib, ceritinib, CDX-301, crizotinib, trametinib, selumetinib, lapatinib, neratinib or sunitinib.

144. The method of claim 136, wherein the mTOR inhibitor comprises everolimus.

145. The method of claim 136, wherein the platinum agent comprises cisplatin, oxaliplatin or carboplatin.

146. The method of claim 136, wherein the B-Raf inhibitor comprises dabrafenib.

147. The method of claim 136, wherein the EGFR inhibitor comprises erlotinib, gefetinib or osimertinib.

148. The method of claim 136, wherein the PARP inhibitor comprises veliparib, olaparib or talazoparib.

149. The method of claim 136, wherein the PI3K inhibitor comprises buparlisib.

150. The method of claim 136, wherein the mitotic inhibitor comprises ixabepilone, paclitaxel or eribulin.

151. The method of claim 136, wherein the CDK inhibitor comprises palbociclib, abemaciclib or ribociclib.

152. The method of claim 136, wherein the antibody therapy comprises APX005M, avelumab, bavituximab, bevacizumab, cixutumab, conatumumab, durvalumab, denosumab, dalotuzumab, ficlatuzumab, figitumumab, fresolimumab, Hu3S193, ipilimumab, MN-14, mapatumuzab, matuzumab, MEDI4736, necitumumab, nivolumab, nimotuzumab, nofetumomab, olaratumab, onartuzumab, pembrolizumab, panitumumab, pertuzumab, racotumomab, ramucirumab, rovalpituzumab, tucotuzumab, tremelimumab, trastuzumab, zalutumumab or a combination thereof.

153. The method of claim 136, wherein the immune checkpoint inhibitor comprises a programmed cell death 1 (PD-1) inhibitor, a CD274 molecule (PD-L1) inhibitor or a cytotoxic T-lymphocyte associated protein 4 (CTLA-4) checkpoint inhibitor.

154. The method of claim 136, wherein the immune checkpoint inhibitor comprises atezolizumab, durvalumab, ipilimumab, tremelimumab or indiximod.

155. A method of reducing a risk of, or treating, lung cancer, colorectal cancer or metastatic breast cancer in a subject, comprising:
(a) identifying a subject who has, or who is at risk of developing, lung cancer colorectal cancer or breast cancer;
(b) obtaining information regarding the subject's single nucleotide polymorphism (SNP) alleles for any of the three or five SNP combinations disclosed in Tables 1-3;
(c) diagnosing the subject as at risk for lung, colorectal, or metastatic breast cancer if the subject has a positive IL-1 genotype pattern obtained in (b) that is the same as any of any of those disclosed as IL-1 positive genotypes in tables 1-3; and
(d) administering an inflammation inhibitor to the subject.

156. The method of claim 1555, wherein the inflammation inhibitor is an IL-1 inhibitor, an IL-6 inhibitor, a GM-CSF inhibitor, or a JAK/STAT inhibitor.
